(19) United States
(12) Patent Application Publication
(10) Pub. No.: US 2026/0209760 A1
(43) Pub. Date: Jul. 23, 2026

(54) METHODS AND COMPOSITIONS FOR MODULATING CELLULAR FACTORS TO INCREASE PRIME EDITING EFFICIENCIES

(71) Applicants: The Broad Institute, Inc., Cambridge, MA (US); President and Fellows of Harvard College, Cambridge, MA (US)

(72) Inventors: David R. Liu, Cambridge, MA (US); Peter J. Chen, Cambridge, MA (US); Xin Gao, Cambridge, MA (US)

(73) Assignees: The Broad Institute, Inc., Cambridge, MA (US); President and Fellows of Harvard College, Cambridge, MA (US)

(21) Appl. No.: 19/142,097

(22) PCT Filed: Dec. 21, 2023
(86) PCT No.: PCT/US2023/085586

§ 371 Date: Jun. 20, 2025

Related U.S. Application Data
(60) Provisional application No. 63/477,155, filed on Dec. 23, 2022.

(51) Int. Cl. C12N 15/113; C12N 9/12; C12N 9/22; C12N 15/11; C12N 15/90

(52) U.S. Cl. CPC C12N 15/113; C12N 9/1276; C12N 9/226; C12N 15/11; C12N 15/907; C12Y 207/07049; C12N 2310/14; C12N 2310/141; C12N 2310/20

(57) ABSTRACT

Aspects of the present disclosure generally relate to systems, compositions, and methods for prime editing with improved editing efficiency and/or reduced indel formation by inhibiting one or more genes of interest while conducting prime editing of a target site. Accordingly, the present disclosure provides systems, compositions and methods for editing a nucleic acid molecule by prime editing that involves contacting a nucleic acid molecule with a prime editor, one or more pegRNAs, and an inhibitor of a gene of interest, thereby installing one or more modifications to the nucleic acid molecule at a target site with increased editing efficiency and/or lower indel formation. The present disclosure further provides polynucleotides for editing a DNA target site by prime editing comprising a nucleic acid sequence encoding a napDNAbp, a polymerase, and an inhibitor of one or more genes of interest, wherein the napDNAbp and polymerase is capable in the presence of a pegRNA of installing one or more modifications in the DNA target site with increased editing efficiency and/or lower indel formation. The disclosure further provides, vectors, cells, and kits comprising the compositions and polynucleotides of the disclosure.

### RELATED APPLICATIONS

This application claims priority under 35 U.S.C. § 119 (e) to U.S. Provisional Application U.S. Ser. No. 63/477,155, filed Dec. 23, 2022, which is incorporated herein by reference.

### GOVERNMENT SUPPORT

This invention was made with government support under Grant Nos. U01AI142756, RM1HG009490, and R35GM118062, awarded by the National Institutes of Health. The government has certain right in the invention.

### REFERENCE TO AN ELECTRONIC SEQUENCE LISTING

The contents of the electronic sequence listing (B119570169WO00-SEQ-JQM.xml; Size: 97,632 bytes; and Date of Creation: Dec. 21, 2023) is herein incorporated by reference in its entirety.

### BACKGROUND OF THE INVENTION

Prime editing (PE) systems allow for the precise editing of genetic material at specific targeted locations. The specificity and programmability of these systems holds promise to accelerate the development of genetic engineering and gene therapies. PE systems permit the insertion, deletion, and modification of DNA. Successive generations of prime editing systems (PE1, PE2, PE3, PE4, and PE5) have been engineered to improve editing efficiency and specificity. The earliest generations of prime editors suffered from low editing efficiency.

The original PE system (herein “PE1”) had an editing efficiency of about 1-5 percent as described by Anzalone et al. “Search-and-replace genome editing without double strand breaks or donor DNA” Nature 576, 2019; 149-157, which is incorporated herein by reference in its entirety. PE1 comprised a Cas9 (H840A) nickase fused to a M-MLV reverse transcriptase (herein “M-MLV RT”) and a prime editing guide RNA (pegRNA). The Cas9 served as a nucleic acid programmable DNA binding protein (napDNAbp). The pegRNA could be programmed with a specific spacer nucleic acid sequence to bind to a complementary binding site nucleic acid sequence on the target DNA. The full pegRNA comprised a spacer sequence, complementary to target DNA sequence (e.g., binding site); a guide RNA (gRNA) core sequence; and a 3′ extension that both encoded the desired edit and provided a primer binding site (PBS) for the reverse transcriptase. The interactions between the programmable spacer sequence of the pegRNA and the complementary binding site sequence on the target DNA in conjunction with interactions between the Cas9 domain and the protospacer adjacent motif (PAM) targeted the PE1-pegRNA complex to bind one strand of a target DNA locus. Upon binding the PE1 Cas9 nickase could nick the opposite strand, exposing a DNA 3′ end. Upstream of the 3′ end, a DNA primer sequence could hybridize to the primer binding site (PBS) of the pegRNA extension. Reverse transcription from the 3′ end of the primer using the RT template of the pegRNA extension generated a 3′ DNA flap that contained the edited sequence and ultimately directed the incorporation of that sequence into the genome. The editing efficiency of PE1 editors was around 1-5 percent.

The PE2 system resulted from mutations in the M-MLV RT of PE1 that improved editing efficiency to about 2 to 25 percent. The improved PE2 system incorporated the following mutations, D200N/L603W/T330P/T306K/W313F into the M-MLV RT in addition to a Cas9 (H840A) nickase. PE2 exhibited enhanced DNA-RNA affinity, enzyme processivity, and thermostability, and increased the editing efficiency by 2-5 fold relative to PE1.

Despite the increased editing efficiency of PE2, the edits inserted using the PE2 machinery remained vulnerable to repair during DNA heteroduplex resolution. To overcome this inherent limitation in PE2, an additional single guide RNA (sgRNA) was added to the PE2 system to give rise to the PE3 system (e.g., Cas9 (H840A)-M-MLV RT (D200N/L603W/T330P/T306K/W313F, a pegRNA, and sgRNA), wherein the sgRNA was designed to match the edited sequence introduced by the pegRNA but not the original allele, and thus directed the Cas9 nickase portion of the fusion protein to nick the unedited strand at a nearby site, opposite the original nick. Nicking the non-edited strand caused the cell's natural repair system to copy the information in the edited strand to the complementary strand, permanently installing the edit. PE3 improved editing efficiencies relative to PE1 (e.g., by 2-fold to 20-fold) and PE2 (e.g., by 1.5-fold to 4.2 fold).

To further improved editor efficiency, the PE4 and PE5 editor systems incorporated a dominant negative MLH1 into the PE2 and PE3 editor systems. A screen of a pool of 476 CRISPRi gene knockdowns involved in DNA repair and associated processes identified multiple genes involved in DNA mismatch repair (MMR) as impeding prime editing and promoting undesired indel byproducts. The genes included MLH1. Inhibition of MLH1 via CRISPRi resulted in increased editor efficiency. Similarly, incorporation of a dominant negative version of the MLH1 protein into the PE2 and PE3 systems resulted in increased editor efficiency. In the PE4 and PE5 prime editing systems, transient expression of a dominant negative MLH1, which served as an MMR-inhibiting protein, enhanced the efficiency of substitution, small insertion, and small deletion prime edits by an average 7.7-fold and 2.0-fold compared to PE2 and PE3 systems, respectively, while improving edit/indel ratios by 3.4-fold in MMR-proficient cell types. However, inclusion of a dominant negative suppressor the MMR pathway may cause problems in certain genetic engineering or therapy situations. In particular, suppression of MMR may result in increased mutations at non-target locations.

The realization that inhibition of mismatch repair increased PE2 and PE3 prime editor efficiency led to the insight that strategic installation of silent mutations, beyond a single base mismatch (the target for MMR pathway) could also enhance prime editing efficiency by evading the MMR repair pathway.

Other efforts to improve editor efficiency included codon optimization in the nucleic acid sequence encoding the Cas9-RT of the PE3 system and optimization of nuclear localization signals and linkers resulting in the PEmax prime editor system that enhanced editing efficacy by 2.8-fold over the PE3 system. (Cite Chen et al., Cell 184, 5635-5652 Oct. 28, 2021)

Early prime editor systems also suffered from the inability to mediate insertions or deletions the size of typical exons or gene coding sequences (e.g., PE2 and PE3 could only make precise insertions of up to ~40 bp and deletions up to ~80 bp). To overcome this limitation, alternative prime editing systems were developed (e.g., Twin Prime (twinPE) and Prime Del (PrimeDel)). TwinPE was designed to bypass DNA repair mechanisms directed at (1) 3′ flap annealing and ligation and (2) heteroduplex resolution. To do this, an additional pegRNA was added to the PE machinery. Both twinPE and PrimeDel comprise a pair of pegRNAs. Each pegRNA targeted a different DNA strand and each served as a template for the synthesis of a 3′ flap with complementary to the 3′ flap templated by the other pegRNA. PrimeDEL differs from TwinPE prime in that each 3′ flap contain a region complementary to the primer from the other 3′ flap. Thus, using the complementarity allows for a perfect deletion between the two primer sites. TwinPE, on the other hand, does not allow for more than one 3′ flap to contain a region of complementarity to the primer from the other 3′ flap. Thus, TwinPE always results in larger insert edits and higher insertion efficiency.

Researchers hypothesized that if the newly synthesized DNA strands were highly dissimilar to the endogenous target site, the complementary 3′ flaps would preferentially hybridize with each other to create an intermediate containing annealed 3′ overhangs of new DNA sequence and annealed 5′ overhangs of original DNA sequence. Because both strands are synthesized by prime editors, there was no requirement for strand invasion of the target site or for the edit to be copied to the complementary DNA strand. Excision of the original DNA sequence (e.g., the 5′ overhangs) and ligation of the pair of nicks resulted in replacement of the endogenous sequence with the paired 3′ flap sequences.

### SUMMARY OF THE INVENTION

The present disclosure describes an improved and modified approach to prime editing comprising inhibiting one or more genes targeting one or more pathways listed in Tables 1-7 during prime editing. The inventors have surprisingly found that the previously identified MMR inhibitors, shown to improve the editing efficiency of PE3 editors, failed to improve the editing efficiency of twinPE or PrimeDel editors. Without being bound by theory, it is believed that the mechanism of action by which the various editors install the desired edits may change the innate repair mechanisms used to reverse the desired edit. It is also believed that while the MMR repair pathway is known to play a key role in repairing base substitution mismatches and insertion-deletion mismatches, other unidentified pathways, may also play a key role in inhibiting PE3 prime editing.

As described herein, the inventors have discovered that other genes involved in chromatin/chromatin-binding, chromatin remodeling, nuclease activity, helicase activity, and DNA damage response also inhibit the editing efficiency of PE3, twinPE, and PrimeDel (see Tables 1-7). Thus, various aspects of the present disclosure relate to inhibiting, blocking, or otherwise inactivating one or more of these genes associated with chromatin/chromatin-binding, chromatin remodeling, nuclease activity, helicase activity, and DNA damage response as listed in Tables 1-7 (e.g., in addition to the MMR pathway).

In some embodiments, the editing efficiency of PE3, twinPE, or PrimeDel may be significantly increased (e.g., by at least 1.3-fold increase, by at least 1.5-fold increase, by at least 1.7-fold increase, by at least 1.9-fold increase, by at least 2.0-fold increase, by at least 2.2 fold increase, by at least 2.4 fold increase, by at least 2.6 fold increase, by at least 2.8 fold increase, by at least 3.0 fold increase, by at least 4.0-fold increase, or by at least 5.0-fold increase) or more) when one or more functions of the one or more genes listed in Tables 1-7 are inhibited, blocked, or otherwise inactivated during prime editing.

In some embodiments, the frequency of indel formation resulting from prime editing may be significantly decreased (e.g., by at least 1.3-fold decrease, by at least 1.5-fold decrease, by at least 1.7-fold decrease, by at least 1.9-fold decrease, by at least 2.0-fold decrease, by at least 2.2 fold decrease, by at least 2.4 fold decrease, by at least 2.6 fold decrease, by at least 2.8 fold decrease, by at least 3.0 fold decrease, by at least 4.0-fold decrease, or by at least 5.0-fold increase decrease or lower) when one or more functions of the one or more genes listed in Tables 1-7 are inhibited, blocked, or otherwise inactivated during prime editing.

Thus, some aspects of the present disclosure relate to compositions comprising inhibitors that enhance the editing efficiency of one or more systems disclosed herein (e.g., PE3, twinPE, or PrimeDel editing systems). In some embodiments, the composition is capable of installing one or more modifications to a nucleic acid molecule at a target site. The disclosure contemplates any suitable means by which to inhibit, block, or otherwise inactivate the one or more genes or gene products listed in Tables 1-7, including, but not limited to, inactivating one or more proteins of the one or more genes at the genetic level, e.g., by introducing one or more mutations in the gene(s) encoding a protein of a gene listed in Tables 1-7. Exemplary genes or gene products that may be inhibited, blocked, or otherwise inactivated include, but are not limited to, CHAF1B, DBR1, XRN2, CCNH, NOP10, MYB, XRCC5, DKC1, TAF1, SMARCA5, GAR1, EXOSC2, NSMCE1, CDK12, RCL1, DHX36, PPP1R8, SMC6, NSMCE4A, MCM2, NONO, ASFIA, SMC5, NSMCE2, RAD51, FIP1L1, and MCM6 (e.g., these genes increase editing efficiency of PE3, twinPE, and Prime Del by at least 1.3 fold, relative to editing in the absence of said inhibitor).

The nucleotide and amino acid sequences of the genes and gene products listed in Tables 1-7 are known in the art. The present disclosure embraces using any known naturally-occurring protein of a gene listed in Tables 1-7, any naturally-occurring variant of a protein of a gene listed in Tables 1-7, any engineered variant (including single or multiple amino acid substitutions, deletions, insertions, rearrangements, or fusions) of a protein of a gene listed in Tables 1-7 for use in the present disclosure so long as the inhibiting, blocking, or otherwise inactivation of one or more of said proteins or variants thereof result in the inhibition, blockage, or inactivation of the one or more genes listed in Tables 1-7. The inhibiting, blocking, or inactivation of any one or more proteins (or variants thereof) of genes listed in Tables 1-7 may use any suitable means applied at the genetic level (e.g., in the gene encoding the one or more CHAF1B proteins, such as introducing a mutation (e.g., using prime editing, siRNA, etc.) that inactivates the CHAF1B protein or variant thereof), transcriptional level (e.g., by transcript knockdown), translational level (e.g., by blocking translation of one or more CHAF1B proteins from their cognate transcripts), post-translational level (e.g., by blocking post-translational modification of a protein product), or protein level (e.g., administering of an inhibitor (e.g., small molecule, antibody or fragment thereof, dominant negative protein variant), or by targeted protein degradation (e.g., PROTAC-based degradation).

In some embodiments, the inhibitor is a CRISPR interference inhibitor. In some embodiments, the inhibitor is an RNA interference inhibitor, for example, a small interfering RNA (siRNA) or a microRNA (miRNA). In other embodiments, the inhibitor is a small molecule inhibitor, for example, a covalent inhibitor or a non-covalent inhibitor. In some embodiments, the inhibitor comprises an antibody or an antibody fragment. Other inhibitors are also possible in other embodiments. For example, in some cases, the inhibitor comprises a dominant negative gene product of one of the genes listed in Tables 1-7.

In some embodiments, a system relates to a composition comprising a twinPE editor and an inhibitor of one or more genes or gene products listed in Table 1 (e.g., twinPE system). In some embodiments, the system comprises a first prime editor comprising a first nucleic acid programmable DNA binding protein (first napDNAbp) and a first polypeptide comprising an RNA-dependent DNA polymerase activity. In some embodiments, the first prime editor further comprises a first prime editing guide RNA (first pegRNA) with a first spacer sequence that binds to a first binding site on a first strand of the double-stranded DNA sequence upstream of the target site to be edited. The system also comprises a second prime editor comprising a second nucleic acid programmable DNA binding protein (second napDNAbp) and a second polypeptide comprising an RNA-dependent DNA polymerase activity. The second prime editor also comprises a second prime editing guide RNA (second pegRNA) with a second spacer sequence that binds to a second binding site on a second strand of the double-stranded DNA sequence upstream of the target site to be edited, in some embodiments. In some embodiments, the system further comprises one or more inhibitors of one or more genes or gene products, wherein inhibiting the one or more genes or gene products increases the editing efficiency of the system by at least 1.3-fold. In some embodiments, the one or more genes that increase the editing efficiency are listed in Table 1. Exemplary genes and/or gene products listed in Table 1 include, but are not limited to, CHAF1B, DBR1, XRN2, GTF2H4, CCNH, MNAT1, NOP10, GTF2F2, CDK7, and DROSHA.

In some embodiments, the first pegRNA comprises a first DNA synthesis template encoding a first single-stranded DNA sequence, and the second pegRNA comprises a second DNA synthesis template encoding a second single-stranded DNA sequence. In some embodiments, the first and the second single-stranded DNA sequence each comprise a region of complementarity to the other. In some embodiments, the first single-stranded DNA sequence and the second single-stranded DNA sequence form a duplex comprising an edited portion as compared to the DNA sequence at the target site to be edited.

In some embodiments, a system (e.g., twinPE system) comprises a first prime editor comprising a first nucleic acid programmable DNA binding protein (first napDNAbp) and a first polypeptide comprising an RNA-dependent DNA polymerase activity; and a first prime editing guide RNA (first pegRNA) comprising a first spacer sequence, a first gRNA core, a first DNA synthesis template, and a first primer binding site, wherein the first primer binding site binds to a first primer on a first strand of the double-stranded DNA sequence upstream of the target site to be edited. In some embodiments, the system comprises a second prime editor comprising a second nucleic acid programmable DNA binding protein (second napDNAbp) and a second polypeptide comprising an RNA-dependent DNA polymerase activity; and a second prime editing guide RNA (second pegRNA) comprising a second spacer sequence, a second gRNA core, a second DNA synthesis template, and a second primer binding site. In some embodiments, the second primer binding site binds to a second primer on a second strand of the double-stranded DNA sequence upstream of the target site to be edited. In some embodiments, the system comprises one or more inhibitors of one or more genes or gene products, wherein inhibiting the one or more genes or gene products increases the editing efficiency of the system by at least 1.3-fold. In some embodiments, the first DNA synthesis template encodes a first single-stranded DNA and the second DNA synthesis template encodes a second single-stranded DNA. In some embodiments, the first single-stranded DNA sequence and the second single-stranded DNA sequence each comprise a region of complementarity to the other. In some embodiments, the first single-stranded DNA sequence does not have a region of complementarity to the second primer. In some embodiments, the second single-stranded DNA sequence does not have a region of complementarity to the first primer. In some embodiments, the first single-stranded DNA sequence and the second single-stranded DNA sequence form a duplex comprising an edited portion as compared to the DNA sequence at the target site to be edited.

In some embodiments, a system relates to a composition comprising a PrimeDel editor and an inhibitor of one or more genes or gene products listed in Table 2 (e.g., a PrimeDel system). In some embodiments, the system comprises a first prime editor comprising a first nucleic acid programmable DNA binding protein (first napDNAbp) and a first polypeptide comprising an RNA-dependent DNA polymerase activity; and a first prime editing guide RNA (first pegRNA) that binds to a first binding site on a first strand of the double-stranded DNA sequence upstream of the target site to be edited. In some embodiments, the system comprises a second prime editor comprising a second nucleic acid programmable DNA binding protein (second napDNAbp) and a second polypeptide comprising an RNA-dependent DNA polymerase activity; and a second prime editing guide RNA (second pegRNA) that binds to a second binding site on a second strand of the double-stranded DNA sequence upstream of the target site to be edited. In some embodiments, the system comprises the one or more inhibitors of one or more genes or gene products, wherein inhibiting the one or more genes or gene products increases the editing efficiency of the system by at least 1.3-fold. In some embodiments, the genes or gene products are listed in Table 2. Exemplary genes and/or gene products include, but are not limited to, CHAF1B, DBR1, XRN2, GTF2H4, CCNH, MNAT1, NOP10, GTF2F2, MYB, ERCC2, HINFP, XRCC5. In some embodiments, the first pegRNA comprises a first DNA synthesis template that comprises a region of complementary to the second binding site of the second pegRNA, and the second pegRNA comprises a second DNA synthesis template that comprises a region of complementary to the first binding site of the first pegRNA.

In other embodiments, still, a system (e.g., a PrimeDel system) comprises a first prime editor comprising a first nucleic acid programmable DNA binding protein (first napDNAbp) and a first polypeptide comprising an RNA-dependent DNA polymerase activity (e.g., a reverse transcriptase); and a first prime editing guide RNA (first pegRNA) comprising a first spacer sequence, a first gRNA core, a first DNA synthesis template, and a first primer binding site that binds to a first binding site on a first strand of the double-stranded DNA sequence upstream of the target site to be edited, or one or more polynucleotides encoding the first pegRNA. In some embodiments, the system further comprises a second prime editor comprising a second prime editor comprising a second nucleic acid programmable DNA binding protein (second napDNAbp) and a second polypeptide comprising an RNA-dependent DNA polymerase activity; and a second prime editing guide RNA (second pegRNA) comprising a second spacer sequence, a second gRNA core, a second DNA synthesis template, and a second primer binding site that binds to a second binding site on a second strand of the double-stranded DNA sequence upstream of the target site to be edited, or one or more polynucleotides encoding the second pegRNA In some embodiments, the system further comprises the one or more inhibitors of one or more genes or gene products, wherein inhibiting the one or more genes or gene products increases the editing efficiency of the system by at least 1.3 fold. In some embodiments, the genes or gene products are listed in Table 2. In some embodiments, the first DNA synthesis template encodes a first single-stranded DNA and the second DNA synthesis template encodes a second single-stranded DNA. In some embodiments, the first single-stranded DNA sequence and the second single-stranded DNA sequence each comprise a region of complementarity to the other. In some embodiments, the first single-stranded DNA sequence has a region of complementarity to the second primer. In some embodiments, the second single-stranded DNA sequence has a region of complementarity to the first primer. In some embodiments, the first single-stranded DNA sequence and the second single-stranded DNA sequence form a duplex comprising an edited portion as compared to the DNA sequence at the target site to be edited.

In some embodiments, a system relates to a composition comprising a PE3 editor and an inhibitor of one or more genes or gene products listed in Table 3 (e.g., a PE3 system). In some embodiments, the system comprises a prime editor, a pegRNA, a sgRNA, and one or more inhibitors of one or more genes or gene products. In some embodiments, inhibiting the one or more genes or gene products increases the editing efficiency of the system by at least 1.3 fold. In some embodiments, the system is capable of installing one or more modification to the nucleic acid molecule at a target site. Exemplary gene and/or gene products listed in Table 3 included, but are not limited to, CHAF1B, DBR1, XRN2, CCNH, NOP10, MYB, XRCC5, DKC1, TAF1, and TSEN2.

In other embodiments, the composition comprises a twinPE system or a PrimeDel system and one or more inhibitors of the genes or gene products listed in Table 4. Exemplary genes and/or gene products listed in Table 4 include, by are not limited to, CHAF1B, DBR1, XRN2, GTF2H4, CCNH, MNAT1, NOP10, GTF2F2, MYB, and ERCC2.

In some embodiments, the composition comprises a twinPE system or a PE3 system and one or more inhibitors of the genes or gene products listed in Table 5. Exemplary genes and/or gene products listed in Table 5 include, by are not limited to, CHAF1B, DBR1, XRN2, CCNH, NOP10, MYB, XRCC5, DKC1, TAF1, and TSEN2.

In some embodiments, the composition comprising a PrimeDel system or a PE3 system and an inhibitor listed in Table 6. Exemplary genes and/or gene products listed in Table 5 include, by are not limited to, CHAF1B, DBR1, XRN2, CCNH, NOP10, MYB, XRCC5, DKC1, TAF1, SMARCA5

In other embodiments, the composition comprises a twinPE, PrimeDel, and PE3 system and one or more inhibitors of genes or gene products listed in Table 7. Exemplary genes and/or gene products listed in Table 5 include, by are not limited to, CHAF1B, DBR1, XRN2, CCNH, NOP10, MYB, XRCC5, DKC1, TAF1, SMARCA5, GAR1, EXOSC2, NSMCE1, CDK12, RCL1, DHX36, PPP1R8, SMC6, NSMCE4A, MCM2, NONO, ASF1A, SMC5, NSMCE, RAD51, FIP1L1, and MCM6.

In some embodiments, the composition comprises a twinPE system and an inhibitor of one or more complexes comprising one or more gene products from one or more genes listed in Table 1. In some embodiments, the composition comprises a PrimeDel system and an inhibitor of one or more complexes comprising one or more gene products from one or more genes listed in Table 2. In some embodiments, the composition comprises a PE3 system and an inhibitor of one or more complexes comprising one or more gene products from one or more genes listed in Table 3. In some embodiments, the composition comprises a twinPE system and an inhibitor of one or more pathways comprising one or more gene products from one or more genes listed in Table 1. In some embodiments, the composition comprises a PrimeDel system and an inhibitor of one or more pathways comprising one or more gene products from one or more genes listed in Table 2. In some embodiments, the composition comprises a PE3 system and an inhibitor of one or more pathways comprising one or more gene products from one or more genes listed in Table 3.

Additional aspects of the present invention relate to polynucleotides encoding one or more systems or compositions disclosed herein. For example, in some embodiments, the polynucleotide encodes of a system (e.g., a twinPE system, PrimeDel system, or PE3 system) and one or more inhibitors of one or more genes listed in Tables 1-7. In some embodiments, the polynucleotide encodes a fusion protein comprising a napDNAbp fused to a RT (e.g., Cas9 (H840A)-MMLV), a pair of pegRNAs and one or more inhibitors of one or more genes or gene products in Tables 1-7. In some cases, the polynucleotide encodes a twinPE system and one or more inhibitors of one or more genes or gene products listed in Table 1. In some embodiments, the polynucleotide encodes a PrimeDel system and one or more inhibitors of one or more genes or gene products listed in Table 2. In some embodiments, the polynucleotide encodes a PE3 system and one or more inhibitors to one or more genes listed in Table 3. In some embodiments, the polynucleotide encodes a twinPE system or a PrimeDel system and one or more inhibitors to one or more genes or gene products listed in Table 4. In some embodiments, polynucleotide encodes a twinPE system or a PE system and one or more inhibitors to one or more genes or gene products listed in Table 5. In some embodiments, polynucleotide encodes a PrimeDel system or a PE system and one or more inhibitors to one or more genes or gene products listed in Table 6. In some embodiments, the polynucleotide encodes a twinPE system, PrimeDel system or a PE system and one or more inhibitors to one or more genes or gene products listed in Table 7. In some embodiments, the polynucleotide encodes for one or more inhibitors of one or more complexes or pathways comprising one or more gene products from one or more genes listed in Table 1-7, in addition to, for example, one or more systems (e.g., PE3 system). In some embodiments, the polynucleotides contemplated herein may encode for any system or part thereof or composition disclosed herein, in addition to, one or more inhibitors of genes, gene products, complexes formed from one or more gene products, or pathways comprising one or more gene products from the one or more genes listed in Tables 1-7.

Other aspects of the disclosure relate to vectors comprising one or more polynucleotides disclosed herein and cells comprising one or more polynucleotides or one or more vectors disclosed herein. In some aspects, the disclosure also relates to pharmaceutical compositions comprising any one of the polynucleotides, vectors, or cells disclosed herein and a pharmaceutical excipient.

Additionally, aspects of the disclosure relate to methods for editing a nucleic acid molecule (e.g., DNA) by prime editing. In some embodiments, the method comprises contacting the nucleic acid with any one of the systems disclosed herein. In other embodiments, the method comprises contacting the nucleic acid with any one of the compositions disclosed herein. In some embodiments, the method comprises contacting the nucleic acid with any one of the polynucleotides disclosed herein. In some embodiments, the method comprises contacting the nucleic acid with any one of the vectors disclosed herein. In some embodiments, the method comprises contacting the nucleic acid with any one of the pharmaceutical compositions disclosed herein.

### BRIEF DESCRIPTION OF THE DRAWINGS

The following drawings form part of the present specification and are included to further demonstrate certain aspects of the present disclosure, which may be better understood by reference to one or more of these drawings in combination with the detailed description of specific embodiments presented herein.

FIG. 1 shows a schematic diagram of the CRISPRi screen pipeline for identifying genetic modulators of prime editing outcomes. The expanded CRISPRi guide library allows for assessment of the impact of more genes/pathways on prime editing and other prime edit types including twin prime editing (TPE), mediated sequence replacement, Prime-DEL mediated deletion, and PE3.

FIG. 2 shows a categorical breakdown of the final gene list described herein, which was generated using the expanded CRISPRi library and summarizes the set of genes perturbed in human cells. Additional, previously untested, DNA damage response proteins were included. Moreover, categories of chromatin/chromatin-binding, chromatin remodeling, nuclease, and helicase were included because of their potential roles in affecting the editing outcomes.

FIGS. 3A-3B shows a comparison of prime edit types that were assayed in the CRISPRi screen described herein. FIG. 3A is a schematic diagram demonstrating TwinPE mediated 50-bp replacement with attB, PrimeDEL mediated 50-bp deletion, and PE3 mediated +6 G to C base substitution. FIG. 3B shows exemplary results of a miseq sequencing analysis illustrating the editing efficiency of TwinPE, PrimeDEL, and PE3 on bulk gDNA.

FIG. 4 is a schematic diagram of an exemplary CRISPRi screen design to evaluate the effect of 1329 gene knockdowns on TwinPE, PrimeDEL, and PE3 editing outcomes.

FIG. 5 shows exemplary fold changes in TwinPE editing efficiency from CRISPRi gene knockdown of DBR1, XRN2, TAF6L, and HELQ in two biological replicates. Results show that knockdown of DBR1 and XRN2 increased TwinPE editing efficiency, and knockdown of TAF6L and HELQ decreased TwinPE editing efficiency. Each dot represents a CRISPRi guide that targets the knockdown of the specified human gene.

FIG. 6 shows fold changes in TwinPE editing efficiency from CRISPRi gene knockdown of genes functioning in Pol II transcription-related pathways in two biological replicates. Knockdown of genes that function in Pol II transcription-related pathways improved TwinPE editing efficiency, including knockdown of CDK activating kinase complex, TFIIH complex, and TFIIF complex. Each dot represents a CRISPRi guide that targets the knockdown of the specified human gene.

FIG. 7 shows fold changes in TwinPE editing efficiency from CRISPRi gene knockdown of DROSHA, TTI1, and TTI2 in two biological replicates. Knockdown of genes DROSHA, TTI1, or TTI2 enhanced TwinPE editing efficiency. Each dot represents a CRISPRi guide that targets the knockdown of the specified human gene.

FIG. 8 shows fold changes in TwinPE editing efficiency from CRISPRi gene knockdown of small nucleolar proteins and PNPT1 in two biological replicates. Knockdown of small nucleolar proteins (DKC1, NOP10, NHP2, GAR1) enhanced TwinPE editing efficiency. Knockdown of PNPT1 decreased TwinPE editing efficiency. Each dot represents a CRISPRi guide that targets the knockdown of the specified human gene.

FIG. 9 shows fold changes in TwinPE editing efficiency from CRISPRi gene knockdown of ribonuclease P complex in two biological replicates. Knockdown of ribonuclease P complex (POP5 and RPP21) increased TwinPE editing efficiency. Each dot represents a CRISPRi guide that targets the knockdown of the specified human gene.

FIG. 10 shows fold changes in PrimeDEL editing efficiency from CRISPRi gene knockdown of DBR1, XRN2, Fanconi anemia, and TAF6L in two biological replicates. Knockdown of DBR1 and XRN2 enhanced PrimeDEL editing efficiency. Similarly, knockdown of Fanconi anemia replated proteins increased PrimeDEL editing efficiency. Knockdown of TAF6L decreased PrimeDEL editing efficiency. Each dot represents a CRISPRi guide that targets the knockdown of the specified human gene.

FIG. 11 shows fold changes in PrimeDEL editing efficiency from CRISPRi gene knockdown of UBE2T and Fanconi 9-1-1 DNA damage checkpoint proteinsin two biological replicates. Knockdown of UBE2T increased PrimeDEL editing efficiency. Similarly, knockdown of Fanconi 9-1-1 DNA damage checkpoint proteins promoted PrimeDEL editing efficiency. Each dot represents a CRISPRi guide that targets the knockdown of the specified human gene.

FIG. 12 shows fold changes in PrimeDEL editing efficiency from CRISPRi gene knockdown of RNAESH1 and ESPL1. in two biological replicates. Knockdown of RNAESH1 or ESPL1 decreased PrimeDEL editing efficiency. Each dot represents a CRISPRi guide that targets the knockdown of the specified human gene.

FIG. 13 shows fold changes in PrimeDEL editing efficiency from CRISPRi gene knockdown of KDM1A in two biological replicates. Knockdown of KDM1A decreased PrimeDEL editing efficiency. Each dot represents a CRISPRi guide that targets the knockdown of the specified human gene.

FIG. 14 shows fold changes in PE3 editing efficiency from CRISPRi gene knockdown of DBR1, XRN2, and mismatch repair proteins in two biological replicates. Knockdown of DBR1 and XRN2 enhanced PE3 editing efficiency. Knockdown of mismatch repair proteins improved PE3 editing efficiency. Knockdown of TAF6L decreased PE3 editing efficiency. Each dot represents a CRISPRi guide that targets the knockdown of the specified human gene.

FIG. 15 shows fold changes in PE3 editing efficiency from CRISPRi gene knockdown of exosome complex and BTAF1 in two biological replicates. Knockdown of exosome complex enhanced PE3 editing efficiency. Knockdown of BTAF1 decreased PE3 editing efficiency. Each dot represents a CRISPRi guide that targets the knockdown of the specified human gene.

FIG. 16 shows fold changes in PE3 editing efficiency from CRISPRi gene knockdown of SMC1A, SMC5, SMC6, and NUDT1 in two biological replicates. Knockdown of SMC1A, SMC5, and SMC6 enhanced PE3 editing efficiency. Knockdown of NUDT1 decreased PE3 editing efficiency. Each dot represents a CRISPRi guide that targets the knockdown of the specified human gene.

FIG. 17 shows the top genetic modulators of TwinPE, PrimeDEL, and PE3.

CRISPRi-screen-identified top hits affected gene editing efficiency in TwinPE, PrimeDEL, and PE3. Knockdown of some gene(s), such as DBR1, affected the editing outcomes for all three edit types. Knockdown of other genes, affected two types of edits, such as RAD17, or only one unique type of edit.

FIG. 18 shows validation of PE3 edits in wild type, KDM1A knock-out (KO), and DXO-KO RPE cells. Specific base substitution edits are labeled in the figure legend.

FIG. 19 shows validation of TwinPE edits in Wildtype and HELQ-KO RPE cells.

FIG. 20 shows validation of PE2 edits in wild type, KDM1A-KO, and DXO-KO RPE cells. Specific base substitution edits are labeled in the figure legend.

FIG. 21 shows validation of PrimeDEL edits in wild type and KDM1A-KO RPE cells.

FIG. 22 shows validation of PE3 edits in HEK293T cells via siRNA knockdown of NOP10. Specific base substitution edits are labeled in the figure legend.

FIG. 23A shows validation experiments confirming decreased PE2 editing efficiency in RPE clonal cells in which KDM1A or DXO has been knocked out.

FIG. 23B shows validation experiments confirming decreased PE3 editing efficiency in RPE clonal cells in which KDM1A or DXO has been knocked out.

FIG. 24 shows validation experiments confirming decreased PrimeDel editing and decreased TwinPE editing in RPE clonal cells in which KDM1A and HELQ have been knocked out.

FIG. 25A shows validation experiments confirming decreased TwinPE editing in KDM1A and DXO knockout HeLa clonal cells and increased TwinPE editing in MNAT1 knockout HeLa clonal cells.

FIG. 25B shows validation experiments confirming decreased PE3 editing in KDM1A and DXO knockout HeLa clonal cells and increased PE3 editing in MNAT1 knockout HeLa clonal cells.

FIG. 25C shows validation experiments confirming decreased PrimeDel editing in KDM1A and DXO knockout HeLa clonal cells and increased PrimeDel editing in MNAT1 knockout HeLa clonal cells.

FIG. 26A demonstrates that overexpression of DXO cDNA in RPE cells improves TwinPE editing of the CCR5 gene.

FIG. 26B demonstrates that overexpression of DXO cDNA in RPE cells improves PE editing of EMX1, +5 G to T.

FIG. 26C demonstrates that overexpression of DXO cDNA in RPE cells improves PrimeDel editing of the HEK3 gene, 90 bp deletion.

FIG. 27A demonstrates that overexpression of TAF6L cDNA in RPE cells improves TwinPE editing of the CCR5 gene.

FIG. 27B demonstrates that overexpression of TAF6L cDNA in RPE cells improves PE editing of EMX1, +5 G to T.

FIG. 27C demonstrates that overexpression of TAF6L cDNA in RPE cells improves PrimeDel editing of the HEK3 gene, 90 bp deletion.

FIG. 28 demonstrates the effective of overexpression of various HELQ variants on TwinPE editing in HeLa cells. As shown in FIG. 28, overexpression of the full-length HELQ or the C-terminal variant increased editing efficiency of TwinPE.

FIG. 29 demonstrates the effective of overexpression of various HELQ variants on PE3 editing in HeLa cells. As shown in FIG. 28, overexpression of the full-length HELQ or the C-terminal variant increased editing efficiency of PE3 at EMX1, +5 G>T.

FIG. 30 illustrates the various engineered HELQ gene constructs used in FIGS. 31-41. The various constructs include the full length HELQ comprising 1101 amino acids, HELQ N-terminal variant comprising 275 (which is helicase-inactive), HELQ C-terminal variant comprising 826 amino acids (which is helicase active), and HELQ C-terminal K365M comprising 1101 amino acids (which is a dead-helicase variant).

FIG. 31A shows the fold change in PE3 editing in HeLa cells, compared to a RFP control, following overexpression of various HELQ variants as a function of sgRNA nick position for HEK4 and FANCF.

FIG. 31B shows the total sequence reads with the percent indels following PE3 editing of HEK4 or FANCF as a function of sgRNA nick position following co-transfection of various HELQ variants. Cotransfection with either the full-length HELQ or C-term (helicase-active) variant exhibited the lowest incidence of indel formation.

FIG. 32 shows sequencing data confirming that HELQ helps PE3 to eliminate sequence insertions and duplication indels during prime editing.

FIG. 33A shows the percentage of total sequence reads having the desired EMX1+5 G>T edit as a function of nicking guide position following PE3 editing in HEK293T cells following co-transfection with either RFP (control), HELQ C-terminal K365M (dead helicase variant, dHELQ), or the C-terminal HELQ variant (HELQ_C).

FIG. 33B shows the percentage of total sequence reads having the desired HEK4+2 G>T edit as a function of nicking guide position following PE3 editing in HEK293T cells following co-transfection with either RFP (control), HELQ C-terminal K365M (dead helicase variant, dHELQ), or the C-terminal HELQ variant (HELQ_C).

FIG. 33C shows the percentage of total sequence reads having the desired FANCF+5 G>T edit as a function of nicking guide position following PE3 editing in HEK293T cells following co-transfection with either RFP (control), HELQ C-terminal K365M (dead helicase variant, dHELQ), or the C-terminal HELQ variant (HELQ_C).

FIG. 33D shows the percentage of total sequence reads having the desired HEK3+2 G>A edit as a function of nicking guide position following PE3 editing in HEK293T cells following co-transfection with either RFP (control), HELQ C-terminal K365M (dead helicase variant, dHELQ), or the C-terminal HELQ variant (HELQ_C).

FIG. 33E shows the percentage of total sequence reads having the desired HEK3+1 CTT insertion edit as a function of nicking guide position following PE3 editing in HEK293T cells following co-transfection with either RFP (control), HELQ C-terminal K365M (dead helicase variant, dHELQ), or the C-terminal HELQ variant (HELQ_C).

FIG. 34A shows the percentage of total sequence reads having the desired EMX1+5 G>T edit as a function of nicking guide position following PE3 editing in HEK293T cells following co-transfection with either RFP (control), MLH1dn, the C-terminal HELQ variant (HELQ_C), or a combination of the MLH1dn and the C-terminal HELQ variant (HELQ_C).

FIG. 34B shows the percentage of total sequence reads having the desired HEK4+2 G>T edit as a function of nicking guide position following PE3 editing in HEK293T cells following co-transfection with either RFP (control), MLH1dn, the C-terminal HELQ variant (HELQ_C), or a combination of the MLH1dn and the C-terminal HELQ variant (HELQ_C).

FIG. 35A shows the fold-change in editing compared to an RFP control as a function the co-expressed HELQ variant for 13 nicking sgRNAs, programmed to guide the nCas9 to nick the non-edited strand downstream from the pegRNA nick site, across 4 genomic sites (HEK4, EMX1, HEK3, FANCF) and 5 different types of edits (+2 G>T, +5 G>T, +1 CTT insertion, +2 G>A)

FIG. 35B shows the fold-change in indels compared to an RFP control as a function the co-expressed HELQ variant for 13 nicking sgRNAs, programmed to guide the nCas9 to nick the non-edited strand downstream from the pegRNA nick site, across 4 genomic sites (HEK4, EMX1, HEK3, FANCF) and 5 different types of edits (+2 G>T, +5 G>T, +1 CTT insertion, +2 G>A)

FIG. 36A shows a schematic illustrating how co-expression with HELQ could be used to improve TwinPE recoding efficiency to address Rett Syndrome disease mutations.

FIG. 36B shows the percentage of sequencing reads having the desired MECP2 edit a function of TwinPE editing when co-expressed with either RFP or HELQ_C.

FIG. 36C shows the fold change in indels compared to RFP controls as a function of TwinPE editing when co-expressed with HELQ_N, HELQ_dHEL, HELQ_Full, HELQ_C, DXO, XRN2, RFP, or GFP. Targeted sequences include AAVS1-2 with attP, ALB with attB, IDS with attB, and AAVS1-1 with attP in HEK293T cells; AAVS1-2 with attP, AAVS1-2 with attP, and CCR5 with Nm60-attP in HeLa cells; and IDS with attB in RPE1 cells.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc), and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered to incorporate aspects of both the crRNA and tracrRNA into a single RNA species. See, e.g., Jinek M., Chylinski K., Fonfara I., Hauer M., Doudna J. A., Charpentier E. Science 337:816-821 (2012), the entire contents of which are hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821 (2012); Qi et al., “Repurposing CRISPR as an RNA-Guided Platform for Sequence-Specific Control of Gene Expression” (2013) Cell. 28; 152 (5): 1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821 (2012); Qi et al., Cell. 28; 152 (5): 1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 2). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 2). In some embodiments, the Cas9 variant comprises a fragment of SEQ ID NO: 2 Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 2). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 2).

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In certain types of CRISPR systems (e.g., type II CRISPR systems), correct processing of pre-crRNA requires a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves a linear or circular nucleic acid target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered to incorporate embodiments of both the crRNA and tracrRNA into a single RNA species—the guide RNA.

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. The tracrRNA of the system is complementary (fully or partially) to the tracr mate sequence present on the guide RNA.

### DNA Synthesis Template

As used herein, the term “DNA synthesis template” refers to the region or portion of the extension arm of a pegRNA that is utilized as a template strand by a polymerase of a prime editor to encode a 3′ single-strand DNA flap that contains the desired edit and which then, through the mechanism of prime editing, replaces the corresponding endogenous strand of DNA at the target site. The extension arm, including the DNA synthesis template, may be comprised of DNA or RNA. In the case of RNA, the polymerase of the prime editor can be an RNA-dependent DNA polymerase (e.g., a reverse transcriptase). In the case of DNA, the polymerase of the prime editor can be a DNA-dependent DNA polymerase. In various embodiments the DNA synthesis template may comprise the “edit template” and the “homology arm”, and all or a portion of the optional 5′ end modifier region, e2. That is, depending on the nature of the e2 region (e.g., whether it includes a hairpin, toeloop, or stem/loop secondary structure), the polymerase may encode none, some, or all of the e2 region as well. Said another way, in the case of a 3′ extension arm, the DNA synthesis template can include the portion of the extension arm that spans from the 5′ end of the primer binding site (PBS) to 3′ end of the gRNA core that may operate as a template for the synthesis of a single-strand of DNA by a polymerase (e.g., a reverse transcriptase). In the case of a 5′ extension arm, the DNA synthesis template can include the portion of the extension arm that spans from the 5′ end of the pegRNA molecule to the 3′ end of the edit template. Preferably, the DNA synthesis template excludes the primer binding site (PBS) of pegRNAs either having a 3′ extension arm or a 5′ extension arm. Certain embodiments described here refer to an “an RT template,” which is inclusive of the edit template and the homology arm, i.e., the sequence of the pegRNA extension arm which is actually used as a template during DNA synthesis. The term “RT template” is equivalent to the term “DNA synthesis template.”

### Edit Template

The term “edit template” refers to a portion of the extension arm that encodes the desired edit in the single strand 3′ DNA flap that is synthesized by the polymerase, e.g., a DNA-dependent DNA polymerase, RNA-dependent DNA polymerase (e.g., a reverse transcriptase). Certain embodiments described here refer to “an RT template,” which refers to both the edit template and the homology arm together, i.e., the sequence of the pegRNA extension arm which is actually used as a template during DNA synthesis. The term “RT edit template” is also equivalent to the term “DNA synthesis template,” but wherein the RT edit template reflects the use of a prime editor having a polymerase that is a reverse transcriptase, and wherein the DNA synthesis template reflects more broadly the use of a prime editor having any polymerase.

### Extension Arm

The term “extension arm” refers to a nucleotide sequence component of a pegRNA which provides several functions, including a primer binding site and an edit template for reverse transcriptase. In some embodiments, the extension arm is located at the 3′ end of the guide RNA. In other embodiments, the extension arm is located at the 5′ end of the guide RNA. In some embodiments, the extension arm also includes a homology arm. In various embodiments, the extension arm comprises the following components in a 5′ to 3′ direction: the homology arm, the edit template, and the primer binding site. Since polymerization activity of the reverse transcriptase is in the 5′ to 3′ direction, the preferred arrangement of the homology arm, edit template, and primer binding site is in the 5′ to 3′ direction such that the reverse transcriptase, once primed by an annealed primer sequence, polymerizes a single strand of DNA using the edit template as a complementary template strand. Further details, such as the length of the extension arm, are described elsewhere herein.

The extension arm may also be described as comprising generally two regions: a primer binding site (PBS) and a DNA synthesis template, for instance. The primer binding site binds to the primer sequence that is formed from the endogenous DNA strand of the target site when it becomes nicked by the prime editor, thereby exposing a 3′ end on the endogenous nicked strand. As explained herein, the binding of the primer sequence to the primer binding site on the extension arm of the pegRNA creates a duplex region with an exposed 3′ end (i.e., the 3′ of the primer sequence), which then provides a substrate for a polymerase to begin polymerizing a single strand of DNA from the exposed 3′ end along the length of the DNA synthesis template. The sequence of the single strand DNA product is the complement of the DNA synthesis template. Polymerization continues towards the 5′ of the DNA synthesis template (or extension arm) until polymerization terminates. Thus, the DNA synthesis template represents the portion of the extension arm that is encoded into a single strand DNA product (i.e., the 3′ single strand DNA flap containing the desired genetic edit information) by the polymerase of the prime editor and which ultimately replaces the corresponding endogenous DNA strand of the target site that sits immediately downstream of the PE-induced nick site. Without being bound by theory, polymerization of the DNA synthesis template continues towards the 5′ end of the extension arm until a termination event. Polymerization may terminate in a variety of ways, including, but not limited to (a) reaching a 5′ terminus of the pegRNA (e.g., in the case of the 5′ extension arm wherein the DNA polymerase simply runs out of template), (b) reaching an impassable RNA secondary structure (e.g., hairpin or stem/loop), or (c) reaching a replication termination signal, e.g., a specific nucleotide sequence that blocks or inhibits the polymerase, or a nucleic acid topological signal, such as, supercoiled DNA or RNA.

### Fusion Protein

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Another example includes a Cas9 or equivalent thereof to a reverse transcriptase. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

Guide RNA (“gRNA”)

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to the spacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353 (6299), the contents of which are incorporated herein by reference. Exemplary sequences are and structures of guide RNAs are provided herein. In addition, methods for designing appropriate guide RNA sequences are provided herein. As used herein, the “guide RNA” may also be referred to as a “traditional guide RNA” to contrast it with the modified forms of guide RNA termed “prime editing guide RNAs” (or “pegRNAs”).

Guide RNAs or pegRNAs may comprise various structural elements that include, but are not limited to:

Spacer sequence—the sequence in the guide RNA or pegRNA (having about 20 nts in length) which binds to the binding site in the target DNA.

gRNA core (or gRNA scaffold or backbone sequence)—refers to the sequence within the gRNA that is responsible for Cas9 binding, it does not include the 20 bp spacer/targeting sequence that is used to guide Cas9 to target DNA.

Extension arm—a single strand extension at the 3′ end or the 5′ end of the pegRNA which comprises a primer binding site and a DNA synthesis template sequence that encodes via a polymerase (e.g., a reverse transcriptase) a single stranded DNA flap containing the genetic change of interest, which then integrates into the endogenous DNA by replacing the corresponding endogenous strand, thereby installing the desired genetic change. Transcription terminator—the guide RNA or pegRNA may comprise a transcriptional termination sequence at the 3′ of the molecule.

### Host Cell

The term “host cell,” as used herein, refers to a cell that can host, replicate, and express a vector described herein, e.g., a vector comprising a nucleic acid molecule encoding an MLH1 variant and a fusion protein comprising a Cas9 or Cas9 equivalent and a reverse transcriptase.

### Linker

The term “linker,” as used herein, refers to a molecule linking two other molecules or moieties. The linker can be an amino acid sequence in the case of a linker joining two fusion proteins. For example, a Cas9 can be fused to a reverse transcriptase by an amino acid linker sequence. The linker can also be a nucleotide sequence in the case of joining two nucleotide sequences together. For example, in the instant case, the traditional guide RNA is linked via a spacer or linker nucleotide sequence to the RNA extension of a prime editing guide RNA which may comprise a RT template sequence and an RT primer binding site. In other embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

napDNAbp

As used herein, the term “nucleic acid programmable DNA binding protein” or “napDNAbp,” of which Cas9 is an example, refer to proteins that use RNA: DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the spacer of a guide RNA). In other words, the guide nucleic-acid “programs” the napDNAbp (e.g., Cas9 or equivalent) to localize and bind to a complementary sequence.

Without being bound by theory, the binding mechanism of a napDNAbp-guide RNA complex, in general, includes the step of forming an R-loop whereby the napDNAbp induces the unwinding of a double-strand DNA target, thereby separating the strands in the region bound by the napDNAbp. The guide RNA spacer then hybridizes to the “target strand.” This displaces a “non-target strand” that is complementary to the target strand, which forms the single strand region of the R-loop. In some embodiments, the napDNAbp includes one or more nuclease activities, which then cut the DNA, leaving various types of lesions. For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”). Exemplary sequences for these and other napDNAbp are provided herein.

### Nickase

The term “nickase” refers to a Cas9 with one of the two nuclease domains inactivated. This enzyme is capable of cleaving only one strand of a target DNA.

### Nucleic Acid Molecule

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, O (6) methylguanine, 4-acetylcytidine, 5-(carboxyhydroxymethyl) uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

pegRNA

As used herein, the terms “prime editing guide RNA” or “pegRNA” or “extended guide RNA” refer to a specialized form of a guide RNA that has been modified to include one or more additional sequences for implementing the prime editing methods and compositions described herein. As described herein, the prime editing guide RNA comprise one or more “extended regions” of nucleic acid sequence. The extended regions may comprise, but are not limited to, single-stranded RNA or DNA. Further, the extended regions may occur at the 3′ end of a traditional guide RNA. In other arrangements, the extended regions may occur at the 5′ end of a traditional guide RNA. In still other arrangements, the extended region may occur at an intramolecular region of the traditional guide RNA, for example, in the gRNA core region which associates and/or binds to the napDNAbp. The extended region comprises a “DNA synthesis template” which encodes (by the polymerase of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA. The extended region may also comprise other functional sequence elements, such as, but not limited to, a “primer binding site” and a “spacer or linker” sequence, or other structural elements, such as, but not limited to aptamers, stem loops, hairpins, toe loops (e.g., a 3′ toeloop), or an RNA-protein recruitment domain (e.g., MS2 hairpin).

As used herein the “primer binding site” comprises a sequence that hybridizes to a single-strand DNA sequence having a 3′end generated from the nicked DNA of the R-loop.

In certain embodiments, the pegRNAs have a 5′ extension arm, a spacer, and a gRNA core. The 5′ extension further comprises in the 5′ to 3′ direction a reverse transcriptase template, a primer binding site, and a linker. The reverse transcriptase template may also be referred to more broadly as the “DNA synthesis template” where the polymerase of a prime editor described herein is not an RT, but another type of polymerase.

In certain other embodiments, the pegRNAs have a 5′ extension arm, a spacer, and a gRNA core. The 5′ extension further comprises in the 5′ to 3′ direction a reverse transcriptase template, a primer binding site, and a linker. The reverse transcriptase template may also be referred to more broadly as the “DNA synthesis template” where the polymerase of a prime editor described herein is not an RT, but another type of polymerase.

In still other embodiments, the pegRNAs have in the 5′ to 3′ direction a spacer (1), a gRNA core (2), and an extension arm (3). The extension arm (3) is at the 3′ end of the pegRNA. The extension arm (3) further comprises in the 5′ to 3′ direction a “primer binding site” (A), an “edit template” (B), and a “homology arm” (C). The extension arm (3) may also comprise an optional modifier region at the 3′ and 5′ ends, which may be the same sequences or different sequences. In addition, the 3′ end of the pegRNA may comprise a transcriptional terminator sequence. These sequence elements of the pegRNAs are further described and defined herein.

In still other embodiments, the pegRNAs have in the 5′ to 3′ direction an extension arm (3), a spacer (1), and a gRNA core (2). The extension arm (3) is at the 5′ end of the pegRNA. The extension arm (3) further comprises in the 3′ to 5′ direction a “primer binding site” (A), an “edit template” (B), and a “homology arm” (C). The extension arm (3) may also comprise an optional modifier region at the 3′ and 5′ ends, which may be the same sequences or different sequences. The pegRNAs may also comprise a transcriptional terminator sequence at the 3′ end. These sequence elements of the pegRNAs are further described and defined herein.

### PE1

As used herein, “PE1” refers to a PE comprising a fusion protein comprising Cas9 (H840A) and a wild type MMLV RT having the following structure: [NLS]-[Cas9 (H840A)]-[linker]-[MMLV_RT(wt)]+a desired pegRNA, wherein the PE fusion has the amino acid sequence of SEQ ID NO: 100, which is shown as follows;

(SEQ ID NO: 100)
MKRTADGSEFESPKKKRKVDKKYSIGLDIGTNSVGWAVITDEYKV
PSKKFKVLGNTDRHSIKKNLIGALLFDSGETAEATRLKRTARRRY
TRRKNRICYLQEIFSNEMAKVDDSFFHRLEESFLVEEDKKHERHP
IFGNIVDEVAYHEKYPTIYHLRKKLVDSTDKADLRLIYLALAHMI
KFRGHFLIEGDLNPDNSDVDKLFIQLVQTYNQLFEENPINASGVD
AKAILSARLSKSRRLENLIAQLPGEKKNGLFGNLIALSLGLTPNF
KSNFDLAEDAKLQLSKDTYDDDLDNLLAQIGDQYADLFLAAKNLS
DAILLSDILRVNTEITKAPLSASMIKRYDEHHQDLTLLKALVRQQ
LPEKYKEIFFDQSKNGYAGYIDGGASQEEFYKFIKPILEKMDGTE
ELLVKLNREDLLRKQRTFDNGSIPHQIHLGELHAILRRQEDFYPF
LKDNREKIEKILTFRIPYYVGPLARGNSRFAWMTRKSEETITPWN
FEEVVDKGASAQSFIERMTNFDKNLPNEKVLPKHSLLYEYFTVYN
ELTKVKYVTEGMRKPAFLSGEQKKAIVDLLFKTNRKVTVKQLKED
YFKKIECFDSVEISGVEDRFNASLGTYHDLLKIIKDKDFLDNEEN
EDILEDIVLTLTLFEDREMIEERLKTYAHLFDDKVMKQLKRRRYT
GWGRLSRKLINGIRDKQSGKTILDFLKSDGFANRNFMQLIHDDSL
TFKEDIQKAQVSGQGDSLHEHIANLAGSPAIKKGILQTVKVVDEL
VKVMGRHKPENIVIEMARENQTTQKGQKNSRERMKRIEEGIKELG
SQILKEHPVENTQLQNEKLYLYYLQNGRDMYVDQELDINRLSDYD
VDAIVPQSFLKDDSIDNKVLTRSDKNRGKSDNVPSEEVVKKMKNY
WRQLLNAKLITQRKFDNLTKAERGGLSELDKAGFIKRQLVETRQI
TKHVAQILDSRMNTKYDENDKLIREVKVITLKSKLVSDFRKDFQF
YKVREINNYHHAHDAYLNAVVGTALIKKYPKLESEFVYGDYKVYD
VRKMIAKSEQEIGKATAKYFFYSNIMNFFKTEITLANGEIRKRPL
IETNGETGEIVWDKGRDFATVRKVLSMPQVNIVKKTEVQTGGFSK
ESILPKRNSDKLIARKKDWDPKKYGGFDSPTVAYSVLVVAKVEKG
KSKKLKSVKELLGITIMERSSFEKNPIDFLEAKGYKEVKKDLIIK
LPKYSLFELENGRKRMLASAGELQKGNELALPSKYVNFLYLASHY
EKLKGSPEDNEQKQLFVEQHKHYLDEIIEQISEFSKRVILADANL
DKVLSAYNKHRDKPIREQAENIIHLFTLTNLGAPAAFKYFDTTID
RKRYTSTKEVLDATLIHQSITGLYETRIDLSQLGGDSGGSSGGSS
GSETPGTSESATPESSGGSSGGSSTLNIEDEYRLHETSKEPDVSL
GSTWLSDFPQAWAETGGMGLAVRQAPLIIPLKATSTPVSIKQYPM
SQEARLGIKPHIQRLLDQGILVPCQSPWNTPLLPVKKPGTNDYRP
VQDLREVNKRVEDIHPTVPNPYNLLSGLPPSHQWYTVLDLKDAFF
CLRLHPTSQPLFAFEWRDPEMGISGQLTWTRLPQGFKNSPTLFDE
ALHRDLADFRIQHPDLILLQYVDDLLLAATSELDCQQGTRALLQT
LGNLGYRASAKKAQICQKQVKYLGYLLKEGQRWLTEARKETVMGQ
PTPKTPRQLREFLGTAGFCRLWIPGFAEMAAPLYPLTKTGTLFNW
GPDQQKAYQEIKQALLTAPALGLPDLTKPFELFVDEKQGYAKGVL
TQKLGPWRRPVAYLSKKLDPVAAGWPPCLRMVAAIAVLTKDAGKL
TMGQPLVILAPHAVEALVKQPPDRWLSNARMTHYQALLLDTDRVQ
FGPVVALNPATLLPLPEEGLQHNCLDILAEAHGTRPDLTDQPLPD
ADHTWYTDGSSLLQEGQRKAGAAVTTETEVIWAKALPAGTSAQRA
ELIALTQALKMAEGKKLNVYTDSRYAFATAHIHGEIYRRRGLLTS
EGKEIKNKDEILALLKALFLPKRLSIIHCPGHQKGHSAEARGNRM
ADQAARKAAITETPDTSTLLIENSSPSGGSKRTADGSEFEPKKKR
KV

### Key:

NUCLEAR LOCALIZATION SEQUENCE (NLS) TOP: (SEQ ID NO: 101), BOTTOM: (SEQ ID NO: 103)
CAS9 (H840A) (SEQ ID NO: 37)
33-AMINO ACID LINKER (SEQ ID NO: 102)
M-MLV reverse transcriptase (SEQ ID NO: 81).

### PE2

As used herein, “PE2” refers to a PE comprising a fusion protein comprising Cas9 (H840A) and a variant MMLV RT having the following structure: [NLS]-[Cas9 (H840A)]-[linker]-[MMLV_RT (D200N) (T330P) (L603W) (T306K) (W313F)]+a desired pegRNA, wherein the PE fusion has the amino acid sequence of SEQ ID NO: 107, which is shown as follows:

(SEQ ID NO: 107)
MKRTADGSEFESPKKKRKVDKKYSIGLDIGTNSVGWAVITDEYKV
PSKKFKVLGNTDRHSIKKNLIGALLFDSGETAEATRLKRTARRRY
TRRKNRICYLQEIFSNEMAKVDDSFFHRLEESFLVEEDKKHERHP
IFGNIVDEVAYHEKYPTIYHLRKKLVDSTDKADLRLIYLALAHMI
KFRGHFLIEGDLNPDNSDVDKLFIQLVQTYNQLFEENPINASGVD
AKAILSARLSKSRRLENLIAQLPGEKKNGLFGNLIALSLGLTPNF
KSNFDLAEDAKLQLSKDTYDDDLDNLLAQIGDQYADLFLAAKNLS
DAILLSDILRVNTEITKAPLSASMIKRYDEHHQDLTLLKALVRQQ
LPEKYKEIFFDQSKNGYAGYIDGGASQEEFYKFIKPILEKMDGTE
ELLVKLNREDLLRKQRTFDNGSIPHQIHLGELHAILRRQEDFYPF
LKDNREKIEKILTFRIPYYVGPLARGNSRFAWMTRKSEETITPWN
FEEVVDKGASAQSFIERMTNFDKNLPNEKVLPKHSLLYEYFTVYN
ELTKVKYVTEGMRKPAFLSGEQKKAIVDLLFKTNRKVTVKQLKED
YFKKIECFDSVEISGVEDRFNASLGTYHDLLKIIKDKDFLDNEEN
EDILEDIVLTLTLFEDREMIEERLKTYAHLFDDKVMKQLKRRRYT
GWGRLSRKLINGIRDKQSGKTILDFLKSDGFANRNFMQLIHDDSL
TFKEDIQKAQVSGQGDSLHEHIANLAGSPAIKKGILQTVKVVDEL
VKVMGRHKPENIVIEMARENQTTQKGQKNSRERMKRIEEGIKELG
SQILKEHPVENTQLQNEKLYLYYLQNGRDMYVDQELDINRLSDYD
VDAIVPQSFLKDDSIDNKVLTRSDKNRGKSDNVPSEEVVKKMKNY
WRQLLNAKLITQRKFDNLTKAERGGLSELDKAGFIKRQLVETRQI
TKHVAQILDSRMNTKYDENDKLIREVKVITLKSKLVSDFRKDFQF
YKVREINNYHHAHDAYLNAVVGTALIKKYPKLESEFVYGDYKVYD
VRKMIAKSEQEIGKATAKYFFYSNIMNFFKTEITLANGEIRKRPL
IETNGETGEIVWDKGRDFATVRKVLSMPQVNIVKKTEVQTGGFSK
ESILPKRNSDKLIARKKDWDPKKYGGFDSPTVAYSVLVVAKVEKG
KSKKLKSVKELLGITIMERSSFEKNPIDFLEAKGYKEVKKDLIIK
LPKYSLFELENGRKRMLASAGELQKGNELALPSKYVNFLYLASHY
EKLKGSPEDNEQKQLFVEQHKHYLDEIIEQISEFSKRVILADANL
DKVLSAYNKHRDKPIREQAENIIHLFTLTNLGAPAAFKYFDTTID
RKRYTSTKEVLDATLIHQSITGLYETRIDLSQLGGDSGGSSGGSS
GSETPGTSESATPESSGGSSGGSSTLNIEDEYRLHETSKEPDVSL
GSTWLSDFPQAWAETGGMGLAVRQAPLIIPLKATSTPVSIKQYPM
SQEARLGIKPHIQRLLDQGILVPCQSPWNTPLLPVKKPGTNDYRP
VQDLREVNKRVEDIHPTVPNPYNLLSGLPPSHQWYTVLDLKDAFF
CLRLHPTSQPLFAFEWRDPEMGISGQLTWTRLPQGFKNSPTLFNE
ALHRDLADFRIQHPDLILLQYVDDLLLAATSELDCQQGTRALLQT
LGNLGYRASAKKAQICQKQVKYLGYLLKEGQRWLTEARKETVMGQ
PTPKTPRQLREFLGKAGFCRLFIPGFAEMAAPLYPLTKPGTLFNW
GPDQQKAYQEIKQALLTAPALGLPDLTKPFELFVDEKQGYAKGVL
TQKLGPWRRPVAYLSKKLDPVAAGWPPCLRMVAAIAVLTKDAGKL
TMGQPLVILAPHAVEALVKQPPDRWLSNARMTHYQALLLDTDRVQ
FGPVVALNPATLLPLPEEGLQHNCLDILAEAHGTRPDLTDQPLPD
ADHTWYTDGSSLLQEGQRKAGAAVTTETEVIWAKALPAGTSAQRA
ELIALTQALKMAEGKKLNVYTDSRYAFATAHIHGEIYRRRGWLTS
EGKEIKNKDEILALLKALFLPKRLSIIHCPGHQKGHSAEARGNRM
ADQAARKAAITETPDTSTLLIENSSPSGGSKRTADGSEFEPKKKR
KV

### Key:

NUCLEAR LOCALIZATION SEQUENCE (NLS) TOP: (SEQ ID NO: 101), BOTTOM: (SEQ ID NO: 103)
CAS9 (H840A) (SEQ ID NO: 37)
33-AMINO ACID LINKER (SEQ ID NO: 102)
M-MLV reverse transcriptase (SEQ ID NO:98).

### PE3

In some embodiments, a prime editing system or composition further comprises a nick guide polynucleotide, such as a nicking guide RNA (ngRNA). Such a PE system or composition may be referred to as a PE3 system or composition. As used herein, “PE3” refers to PE2 plus a second-strand nicking guide RNA that is capable of directing a prime editor to introduce a nick in the non-edited DNA strand (in other words, the target strand of the pegRNA in a PE2 system) in order to induce preferential replacement of the edited strand. In some embodiments, a ngRNA comprises a spacer (referred to as a ngRNA spacer or ng spacer) and a gRNA core, wherein the spacer of the ngRNA comprises a region of complementarity to the edited strand (i.e. the non-target strand of the pegRNA), and wherein the gRNA core can interact with a Cas, e.g., Cas9, of a prime editor. Without wishing to be bound by any particular theory, an ngRNA may bind to the edited strand and direct the Cas nickase to generate a nick on the non-edit strand (or target strand of the pegRNA). In some embodiments, the nick on the non-edited strand directs endogenous DNA repair machinery to use the edited strand as a template for repair of the non-edit strand, which may increase efficiency of prime editing. In some embodiments, the non-edit strand is nicked by a prime editor localized to the non-edit strand by the ngRNA.

### Polymerase

As used herein, the term “polymerase” refers to an enzyme that synthesizes a nucleotide strand and that may be used in connection with the prime editor systems described herein. The polymerase can be a “template-dependent” polymerase (i.e., a polymerase that synthesizes a nucleotide strand based on the order of nucleotide bases of a template strand). The polymerase can also be a “template-independent” polymerase (i.e., a polymerase that synthesizes a nucleotide strand without the requirement of a template strand). A polymerase may also be further categorized as a “DNA polymerase” or an “RNA polymerase.” In various embodiments, the prime editor system comprises a DNA polymerase. In various embodiments, the DNA polymerase can be a “DNA-dependent DNA polymerase” (i.e., whereby the template molecule is a strand of DNA). In such cases, the DNA template molecule can be a pegRNA, wherein the extension arm comprises a strand of DNA. In such cases, the pegRNA may be referred to as a chimeric or hybrid pegRNA which comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the pegRNA is RNA, i.e., including an RNA extension. The term “polymerase” may also refer to an enzyme that catalyzes the polymerization of nucleotide (i.e., the polymerase activity). Generally, the enzyme will initiate synthesis at the 3′-end of a primer annealed to a polynucleotide template sequence (e.g., such as a primer sequence annealed to the primer binding site of a pegRNA) and will proceed toward the 5′ end of the template strand. A “DNA polymerase” catalyzes the polymerization of deoxynucleotides. As used herein in reference to a DNA polymerase, the term DNA polymerase includes a “functional fragment thereof”. A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

### Prime Editing

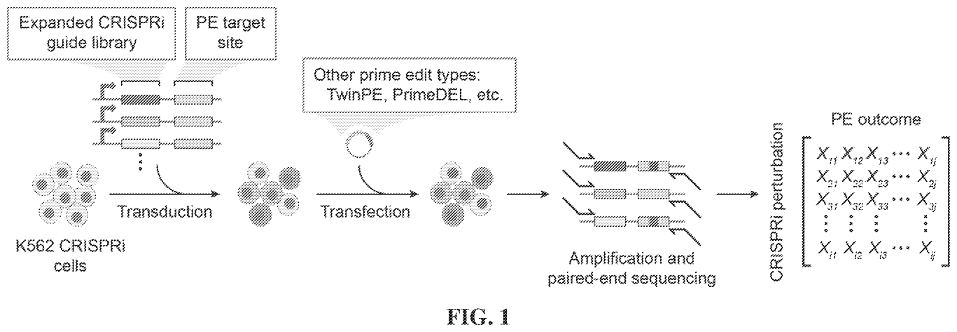

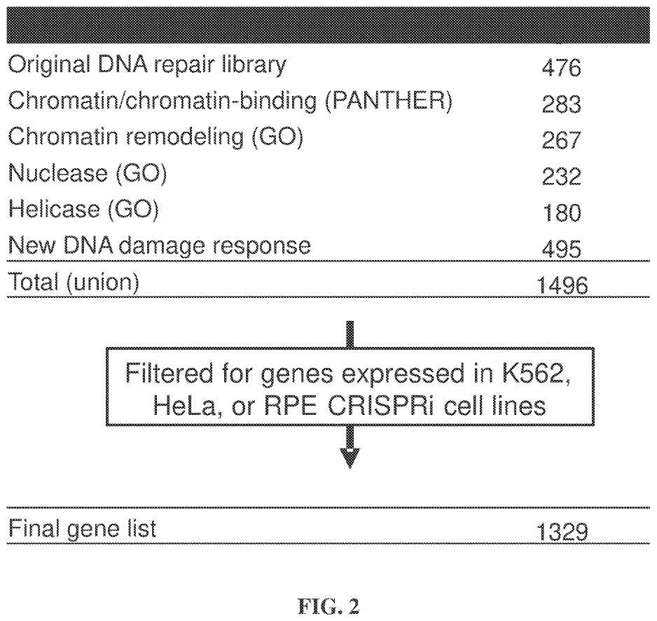

As used herein, the term “prime editing” refers to an approach for gene editing using napDNAbps, a polymerase (e.g., a reverse transcriptase), and specialized guide RNAs that include a DNA synthesis template for encoding desired new genetic information (or deleting genetic information) that is then incorporated into a target DNA sequence. Certain embodiments of prime editing are described in the embodiments of FIG. 1. Classical prime editing is described in the inventors publication of Anzalone, A. V. et al. Search-and-replace genome editing without double-strand breaks or donor DNA. Nature 576, 149-157 (2019), which is incorporated herein by reference in its entirety.

Prime editing represents a platform for genome editing that is a versatile and precise genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“pegRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same (or is homologous to) sequence as the endogenous strand (immediately downstream of the nick site) of the target site to be edited (with the exception that it includes the desired edit). Through DNA repair and/or replication machinery, the endogenous strand downstream of the nick site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit which is installed in place of the corresponding target site endogenous DNA strand. The prime editors of the present disclosure relate, in part, to the discovery that the mechanism of target-primed reverse transcription (TPRT) or “prime editing” can be leveraged or adapted for conducting precision CRISPR/Cas-based genome editing with high efficiency and genetic flexibility. TPRT is naturally used by mobile DNA elements, such as mammalian non-LTR retrotransposons and bacterial Group II introns. The inventors have herein used Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. However, while the concept begins with prime editors that use reverse transcriptase as the DNA polymerase component, the prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, where ever the specification mentions a “reverse transcriptase,” the person having ordinary skill in the art should appreciate that any suitable DNA polymerase may be used in place of the reverse transcriptase. Thus, in one aspect, the prime editors may comprise Cas9 (or an equivalent napDNAbp) which is programmed to target a DNA sequence by associating it with a specialized guide RNA (i.e., pegRNA) containing a spacer sequence that anneals to a complementary binding site in the target DNA. The specialized guide RNA also contains new genetic information in the form of an extension that encodes a replacement strand of DNA containing a desired genetic alteration which is used to replace a corresponding endogenous DNA strand at the target site. To transfer information from the pegRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on pegRNA directly into the target site. In various embodiments, the extension—which provides the template for polymerization of the replacement strand containing the edit—can be formed from RNA or DNA. In the case of an RNA extension, the polymerase of the prime editor can be an RNA-dependent DNA polymerase (such as, a reverse transcriptase). In the case of a DNA extension, the polymerase of the prime editor may be a DNA-dependent DNA polymerase. The newly synthesized strand (i.e., the replacement DNA strand containing the desired edit) that is formed by the herein disclosed prime editors would be homologous to the genomic target sequence (i.e., have the same sequence as) except for the inclusion of a desired nucleotide change (e.g., a single nucleotide change, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. In certain embodiments, the system can be combined with the use of an error-prone reverse transcriptase enzyme (e.g., provided as a fusion protein with the Cas9 domain, or provided in trans to the Cas9 domain). The error-prone reverse transcriptase enzyme can introduce alterations during synthesis of the single strand DNA flap. Thus, in certain embodiments, error-prone reverse transcriptase can be utilized to introduce nucleotide changes to the target DNA. Depending on the error-prone reverse transcriptase that is used with the system, the changes can be random or non-random. Resolution of the hybridized intermediate (comprising the single strand DNA flap synthesized by the reverse transcriptase hybridized to the endogenous DNA strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide change as a result of cellular DNA repair and/or replication processes. Because templated DNA synthesis offers single nucleotide precision for the modification of any nucleotide, including insertions and deletions, the scope of this approach is very broad and could foreseeably be used for myriad applications in basic science and therapeutics.

In various embodiments, prime editing operates by contacting a target DNA molecule (for which a change in the nucleotide sequence is desired to be introduced) with a nucleic acid programmable DNA binding protein (napDNAbp) complexed with a prime editing guide RNA (pegRNA). In various embodiments, the prime editing guide RNA (pegRNA) comprises an extension at the 3′ or 5′ end of the guide RNA, or at an intramolecular location in the guide RNA and encodes the desired nucleotide change (e.g., single nucleotide change, insertion, or deletion). In step (a), the napDNAbp/extended gRNA complex contacts the DNA molecule and the extended gRNA guides the napDNAbp to bind to a target locus. In step (b), a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to the spacer of the extended gRNA) or the “non-target strand” (i.e., the strand forming the single-stranded portion of the R-loop and which is complementary to the target strand). In step (c), the 3′ end of the DNA strand (formed by the nick) interacts with the extended portion of the guide RNA in order to prime reverse transcription (i.e., “target-primed RT”). In certain embodiments, the 3′ end DNA strand hybridizes to a specific RT priming sequence on the extended portion of the guide RNA, i.e., the “reverse transcriptase priming sequence” or “primer binding site” on the pegRNA. In step (d), a reverse transcriptase (or other suitable DNA polymerase) is introduced which synthesizes a single strand of DNA from the 3′ end of the primed site towards the 5′ end of the prime editing guide RNA. The DNA polymerase (e.g., reverse transcriptase) can be fused to the napDNAbp or alternatively can be provided in trans to the napDNAbp. This forms a single-strand DNA flap comprising the desired nucleotide change (e.g., the single base change, insertion, or deletion, or a combination thereof) and which is otherwise homologous to the endogenous DNA at or adjacent to the nick site. In step (e), the napDNAbp and guide RNA are released. Steps (f) and (g) relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the endogenous DNA sequence. Without being bound by theory, the cells endogenous DNA repair and replication processes resolves the mismatched DNA to incorporate the nucleotide change(s) to form the desired altered product. The process can also be driven towards product formation with “second strand nicking.” This process may introduce at least one or more of the following genetic changes: transversions, transitions, deletions, and insertions.

The term “prime editor (PE) system” or “prime editor (PE)” or “PE system” or “PE editing system” refers the compositions involved in the method of genome editing using target-primed reverse transcription (TPRT) describe herein, including, but not limited to the napDNAbps, reverse transcriptases, fusion proteins (e.g., comprising napDNAbps and reverse transcriptases), prime editing guide RNAs, and complexes comprising fusion proteins and prime editing guide RNAs, as well as accessory elements, such as second strand nicking components (e.g., second strand sgRNAs) and 5′ endogenous DNA flap removal endonucleases (e.g., FEN1) for helping to drive the prime editing process towards the edited product formation.

Although in the embodiments described thus far the pegRNA constitutes a single molecule comprising a guide RNA (which itself comprises a spacer sequence and a gRNA core or scaffold) and a 5′ or 3′ extension arm comprising the primer binding site and a DNA synthesis template, the pegRNA may also take the form of two individual molecules comprised of a guide RNA and a trans prime editor RNA template (tPERT), which essentially houses the extension arm (including, in particular, the primer binding site and the DNA synthesis domain) and an RNA-protein recruitment domain (e.g., MS2 aptamer or hairpin) in the same molecule which becomes co-localized or recruited to a modified prime editor that comprises a tPERT recruiting protein (e.g., MS2cp protein, which binds to the MS2 aptamer).

### Prime Editor

The term “prime editor” refers to fusion constructs comprising a napDNAbp (e.g., Cas9 nickase) and a reverse transcriptase and is capable of carrying out prime editing on a target nucleotide sequence in the presence of a pegRNA (or “extended guide RNA”). The term “prime editor” may refer to the fusion protein or to the fusion protein complexed with a pegRNA, and/or further complexed with a second-strand nicking sgRNA. In some embodiments, the prime editor may also refer to the complex comprising a fusion protein (reverse transcriptase fused to a napDNAbp), a pegRNA, and a regular guide RNA capable of directing the second-site nicking step of the non-edited strand as described herein.

### Primer Binding Site

The term “primer binding site” or “the PBS” refers to the nucleotide sequence located on a pegRNA as a component of the extension arm (typically at the 3′ end of the extension arm) and serves to bind to the primer sequence that is formed after Cas9 nicking of the target sequence by the prime editor. A PBS may be a single-stranded portion of the pegRNA that comprises a region of complementarity to the non-target strand (i.e. the PAM strand that has specific PAM sequence adjacent to a protospacer sequence). In some embodiments, the PBS is complementary or substantially complementary to a sequence on the non-target strand of the double stranded target DNA that is immediately upstream of a nick site specific to the prime editor. For example, in some embodiments, the prime editor comprises a Cas9 nickase (e.g. SpCas9 H840A nickase), and the nick site is three nucleotides upstream of the PAM sequence. In some embodiments, in the process of prime editing, the pegRNA complexes with and directs a prime editor to bind a binding site on the target strand of the double stranded target DNA, and generates a nick at the nick site on the non-target strand of the double stranded target DNA. In some embodiments, the PBS is complementary to or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis. In some embodiments, when the Cas9 nickase component of a prime editor nicks one strand of the target DNA sequence, a 3′-ended ssDNA flap is formed, which serves a primer sequence that anneals to the primer binding site on the pegRNA to prime reverse transcription.

### Protospacer

As used herein, the term “protospacer” refers to the sequence (~20 bp) in DNA adjacent to the PAM (protospacer adjacent motif) sequence. The protospacer shares the same sequence as the spacer sequence of the guide RNA. The guide RNA anneals to the complement of the protospacer sequence on the target DNA (specifically, one strand thereof, i.e., the “target strand” versus the “non-target strand” of the target DNA sequence). Such complement sequence of the protospacer sequence may be referred to as a binding site, to which the spacer sequence of the guide RNA is complementary and capable of binding. In order for Cas9 to function it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the target sequence in the genomic DNA, on the non-target strand.

### Protospacer Adjacent Motif (PAM)

As used herein, the term “protospacer adjacent sequence” or “PAM” refers to an approximately 2-6 base pair DNA sequence that is an important targeting component of a Cas9 nuclease. Typically, the PAM sequence is on either strand, and is downstream in the 5′ to 3′ direction of the Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes alternative PAM sequence.

For example, with reference to the canonical SpCas9 amino acid sequence is SEQ ID NO: 2, the PAM sequence can be modified by introducing one or more mutations, including (a) D1135V, R1335Q, and T1337R “the VQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These are examples and are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10 (5): 891-899 (which is incorporated herein by reference).

### Reverse Transcriptase

The term “reverse transcriptase” describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA which can then be cloned into a vector for further manipulation. Avian myoblastosis virus (AMV) reverse transcriptase was the first widely used RNA-dependent DNA polymerase (Verma, Biochim. Biophys. Acta 473:1 (1977)). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNase H activity has been presented by Berger et al., Biochemistry 22:2365-2372 (1983). Another reverse transcriptase which is used extensively in molecular biology is reverse transcriptase originating from Moloney murine leukemia virus (M-MLV). See, e.g., Gerard, G. R., DNA 5:271-279 (1986) and Kotewicz, M. L., et al., Gene 35:249-258 (1985). M-MLV reverse transcriptase substantially lacking in RNase H activity has also been described. See, e.g., U.S. Pat. No. 5,244,797. The invention contemplates the use of any such reverse transcriptases, or variants or mutants thereof.

In addition, the invention contemplates the use of reverse transcriptases that are error-prone, i.e., that may be referred to as error-prone reverse transcriptases or reverse transcriptases that do not support high fidelity incorporation of nucleotides during polymerization. During synthesis of the single-strand DNA flap based on the RT template integrated with the guide RNA, the error-prone reverse transcriptase can introduce one or more nucleotides which are mismatched with the RT template sequence, thereby introducing changes to the nucleotide sequence through erroneous polymerization of the single-strand DNA flap. These errors introduced during synthesis of the single strand DNA flap then become integrated into the double strand molecule through hybridization to the corresponding endogenous target strand, removal of the endogenous displaced strand, ligation, and then through one more round of endogenous DNA repair and/or sequencing processes.

### Reverse Transcription

As used herein, the term “reverse transcription” indicates the capability of an enzyme to synthesize a DNA strand (that is, complementary DNA or cDNA) using RNA as a template. In some embodiments, the reverse transcription can be “error-prone reverse transcription,” which refers to the properties of certain reverse transcriptase enzymes which are error-prone in their DNA polymerization activity.

### Protein, Peptide, and Polypeptide

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

### Spacer Sequence

As used herein, the term “spacer sequence” in connection with a guide RNA or a pegRNA refers to the portion of the guide RNA or pegRNA of about 20 nucleotides which contains a nucleotide sequence that shares the same sequence as the protospacer sequence in the target DNA sequence. The spacer sequence anneals to the complement of the protospacer sequence to form a ssRNA/ssDNA hybrid structure at the target site and a corresponding R loop ssDNA structure of the endogenous DNA strand.

### Target Site

The term “target site” refers to a sequence within a nucleic acid molecule that is edited by a prime editor (PE) disclosed herein. The target site further refers to the sequence within a nucleic acid molecule to which a complex of the prime editor (PE) and gRNA binds.

### Variant

As used herein the term “variant” should be taken to mean the exhibition of qualities that have a pattern that deviates from what occurs in nature, e.g., a variant Cas9 is a Cas9 comprising one or more changes in amino acid residues as compared to a wild type Cas9 amino acid sequence. The term “variant” encompasses homologous proteins having at least 75%, or at least 80%, or at least 85%, or at least 90%, or at least 95%, or at least 99% percent identity with a reference sequence and having the same or substantially the same functional activity or activities as the reference sequence. The term also encompasses mutants, truncations, or domains of a reference sequence, and which display the same or substantially the same functional activity or activities as the reference sequence.

### Vector

The term “vector,” as used herein, refers to a nucleic acid that can be modified to encode a gene of interest and that is able to enter into a host cell, mutate and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### DETAILED DESCRIPTION

Aspects of the present disclosure generally relate to systems, compositions, uses, and methods for prime editing with improved editing efficiency and/or reduced indel formation by inhibiting one or more genes of interest while conducting prime editing of a target site. Accordingly, the present disclosure provides systems, compositions, and methods for editing a nucleic acid molecule by prime editing that involves contacting a nucleic acid molecule with a prime editor, one or more pegRNAs, and an inhibitor of a gene of interest, thereby installing one or more modifications to the nucleic acid molecule at a target site with increased editing efficiency and/or lower indel formation. The present disclosure further provides polynucleotides for editing a DNA target site by prime editing comprising a nucleic acid sequence encoding a napDNAbp, a polymerase, and an inhibitor of one or more genes of interest, wherein the napDNAbp and polymerase in the presence of a pegRNA is capable of installing one or more modifications in the DNA target site with increased editing efficiency and/or lower indel formation. The disclosure further provides nucleic acids, vectors, complexes (e.g., ribonucleoproteins), cells, and kits comprising the compositions and polynucleotides of the disclosure.

Without wishing to be bound by any particular theory, prime editing of a single nucleic acid strand proceeds through a presumed multi-step editing process: 1) the Cas9 domain binds and nicks the target genomic DNA site, which is specified by the pegRNA's spacer sequence; 2) the reverse transcriptase domain uses the nicked genomic DNA as a primer to initiate the synthesis of an edited DNA strand using an engineered extension on the pegRNA as a template for reverse transcription—this generates a single-stranded 3′ flap containing the edited DNA sequence; 3) cellular DNA repair resolves the 3′ flap intermediate by the displacement of a 5′ flap species that occurs via invasion by the edited 3′ flap, excision of the 5′ flap containing the original DNA sequence, and ligation of the new 3′ flap to incorporate the edited DNA strand, forming a heteroduplex of one edited and one unedited strand; and 4) cellular DNA repair replaces the unedited strand within the heteroduplex using the edited strand as a template for repair, completing the editing process.

In some embodiments, systems, compositions, uses, and methods disclosed herein further comprise an additional pegRNA to simultaneously edit a first and a second complementary strand of a double stranded DNA sequence at a target site (e.g., via twinPE system or PrimeDel system). In some embodiments, the edit comprises one or more insertions, deletion, or a combination thereof. Other edits are also possible in other embodiments. For example, in some embodiments, the one or more edits to the nucleic acid molecule installed at the target site comprise one or more transitions, one or more transversions, one or more insertions, one or more deletions, and/or one more inversions.

In some embodiments, including a second pegRNA permits formation of two prime editors (e.g., a first prime editor and a second prime editor) capable in installing large deletions and/or insertions (e.g., greater than or equal to 100 bp). In some embodiments, the first prime editor comprises (1) a first prime editor (e.g., PE2) comprising a first nucleic acid programmable DNA binding protein (napDNAbp) and a first polypeptide comprising an RNA-dependent DNA polymerase activity and (2) a first prime editing guide RNA (first pegRNA) that binds to a first binding site on the first strand of the genomic DNA sequence upstream of the target site. Similarly, the second prime editor comprises, according to some embodiments, (1) a second prime editor (e.g., PE2) comprising a second nucleic acid programmable DNA binding protein (second napDNAbp) and a second polypeptide comprising an RNA-dependent DNA polymerase activity; and a second prime editing guide RNA (second pegRNA) that binds to a second binding site on the second strand of the genomic DNA sequence downstream of the target site.

In some embodiments, the first prime editor causes a first nick at a sequence complementary to the first binding site and the subsequent polymerization of a first single-stranded DNA sequence having a 3′-end (e.g., 3′ flap) from the available 5′-end formed by the first nick. In some embodiments, the second prime editor causes a second nick at a sequence complementary to the second binding site and the subsequent polymerization of a second single-stranded DNA sequence having a 3′-end (e.g., 3′ flap) from the available 5′-end formed by the second nick. In some embodiments, the first single-stranded DNA sequence and the second single-stranded DNA sequence are reverse complements over at least a region of complementarity and form a duplex comprising an edit. In some embodiments, the duplex replaces the nicked first and second complementary strands of the double-stranded DNA sequence. Without wishing to be bound by theory, it is generally believed that complementary 3′ flaps would preferentially hybridize with each other to create an intermediate comprising the annealed 3′ overhang of the edited DNA sequence and an annealed 5′ overhang comprising the original DNA sequence.

In other embodiments, the first single-stranded DNA sequence comprises complementarity to the second binding site (e.g., binding site sequence that is complimentary to the second pegRNA spacer sequence) and the second single-stranded DNA sequence comprises complementarity to the first binding site (e.g., binding site sequence that is complimentary to the first pegRNA spacer sequence). Without being bound by theory, it is generally believed that first 3′ flap would hybridize with the second binding site and the second 3′ flap would hybridize with the first binding site, thus forming an intermediate containing annealed 3′ overhangs comprising the original DNA minus a “deleted” segment of DNA and an annealed 5′ overhang comprising the “deleted” segment of DNA. Additionally, in some embodiments, the first 3′ overhang may include one or more regions of complimentary with the second 3′ overhang (e.g., deletion plus insertion using the PrimeDel system). For example, in some cases a first segment of the first 3′ flap is complimentary to a first segment of the second 3′ flap, a second segment of the first 3′ flap is homologous to the second binding site, and the second segment of the second 3′ flap is homologous to the first binding site.

It has now been discovered that certain genes and gene products involved in MMR, chromatin/chromatin-binding, chromatin remodeling, nuclease activity, helicase activity, and DNA damage response, which when knocked out or knocked down, increase the editing efficiency of the PE3, twinPE, and PrimeDel prime editing systems (e.g., they act as endogenous inhibitors of prime editing). Thus, various aspects of the present disclosure relate to inhibiting, blocking, or otherwise inactivating one or more of the genes associated with chromatin/chromatin-binding, chromatin remodeling, nuclease activity, helicase activity, and DNA damage response as listed in Tables 1-7 (e.g., in addition to the MMR pathway). In some embodiments, the one or more inhibitors of the one or more genes or gene products listed in Tables 1-7 do not include the MMR pathway.

Those of skill in the art will understand that the phrases MMR pathway, chromatin remodeling, nuclease activity, helicase activity, and DNA damage response chromatin/chromatin-binding may encompass different genes depending on the classification system. Exemplary classification systems known in the art include, but are not limited to, GENEOntology, PANTHER, DAVID, Reactome, and KEGG. In some embodiments, the genes disclosed herein were identified using a combination of the GENEOntology and PANTHER databases. Thus, the genes and gene products disclosed herein represent a subset of potential genes or gene products capable of inhibiting prime editing and other genes or gene products involved in MMR pathway, chromatin remodeling, nuclease activity, helicase activity, DNA damage response, and chromatin/chromatin-binding capable of decreasing prime editing efficacy are possible in other embodiments.

Any suitable inhibitor capable of inhibiting blocking, or otherwise inactivating one or more of the genes or gene products associated with MMR, chromatin/chromatin-binding, chromatin remodeling, nuclease activity, helicase activity, and DNA damage response as listed in Tables 1-7, are herein contemplated. The inhibition may involve inhibiting the gene product (e.g., protein) with an inhibitor (e.g., antibody, small molecule inhibitor, or a dominant negative variant of the protein which disrupts, blocks, or otherwise inactivates the function of the protein, e.g., a dominant negative form of CHAF1B). The inhibition may also involve any other suitable means, such as by protein degradation (e.g., PROTAC-based degradation of CHAF1B), transcript-level inhibition (e.g., siRNA transcript degradation/gene silencing or microRNA-based inhibition of translation of the CHAF1B transcript), or at the genetic level (i.e., installing a mutation in the CHAFIB gene (or regulatory regions) which inactivates or reduces the expression of the CHAFIB gene, or which installs a mutation which inactivates, blocks, or minimizes that activity of the encoded CHAF1B product). In addition, the disclosure contemplates that the prime editor (e.g., delivered as a fusion protein comprising a napDNAbp and a polymerase, such as a Cas9 nickase fused to a reverse transcriptase) may be administered together with the inhibitor.

In some embodiments, the inhibitor is a CRISPR inhibitor (herein “CRISPRi”). CRISPRi is an art recognized genetic perturbation technique that allows for sequence-specific repression of gene expression in cells. Without wishing to be bound by theory, it is believed that CRISPRi sterically represses transcription by blocking either transcriptional initiation or elongation, which is accomplished by designing sgRNAs complementary to the promoter or the exonic sequences. Because CRISPRi uses a catalytically inactivated Cas9 (e.g., dead Cas9) that can not cleave the dsDNA but does retain the ability to bind and target sequences within the dsDNA, CRISPRi constitutes a transcriptional equivalent of RNAi (e.g., RNAi operates on the mRNA level or translational level).

In some embodiments, the inhibitor is an RNA interference inhibitor (herein “RNAi”). RNAi is an art recognized technique used to knock down a gene or genes of interest, thus reducing the expression of said gene or genes. Without wishing to be bound by theory, it is believed that RNAi achieves gene knockdown by using a double stranded small interfering RNA (siRNA) that comprises a sequence complementary to the gene of interest. The RNAi cascade begins once the Dicer enzyme processes the siRNA, resulting in the degradation of mRNA and destroys any instructions needed to build certain proteins.

In some embodiments, the RNAi is a microRNA (miRNA). miRNAs are small, single stranded, non-coding RNA molecules. Without being bound by theory, it is believed that miRNAs base-pair to complementary sequences in mRNA molecules, then gene silence the mRNA molecule by one or more of the following processes: (1) cleavage of the mRNA strand into two pieces, (2) destabilization of mRNA by shortening its poly (A) tail, or (3) translation of mRNA into proteins.

In some embodiments, the inhibitor comprises a small molecule inhibitor. In some embodiments, any suitable small molecule inhibitor known in the art may be used herein. covalent inhibitor. In some embodiments, the small molecule inhibitor comprises a covalent inhibitor. Covalent inhibitors are art recognized inhibitors that are rationally designed to bind to and bond with their target protein. Without being bound by theory, it is believed that these inhibitors possess a bond-forming functional group of low chemical reactivity that, following binding to the target protein, is positioned to react rapidly with a proximate nucleophilic residue at the target site to form a bond. In some embodiments, covalent inhibitors are synonymous with irreversible inhibitors.

In some embodiments, the inhibitor is a non-covalent inhibitor. Non-covalent inhibitors are art recognized inhibitors that bind to sites on a substrate via non-covalent interaction such as hydrogen bonds, hydrophobic interactions, and ionic bonds. Non-covalent inhibitors, may in some cases, be competitive inhibitors, uncompetitive inhibitors, non-competitive inhibitors, or mixed. In some embodiments, a competitive inhibitor competes with the native ligand for the active binding site on the substrate. In some embodiments, uncompetitive inhibitors bind to ligand-substrate complexes. In other embodiments, non-competitive inhibitors bind to the target at a site different than the active binding site.

In some embodiments, the inhibitor comprises an antibody or fragment thereof. In some embodiments, the antibody or fragment thereof comprises a fragment antigen binding region (herein “Fab region”). The Fab is an art recognized protein composed of one constant and one variable domain of each of the heavy and light chains. The variable domain contains the paratope (e.g., the antigen binding site) which comprises a set of complementarity-determining regions at the amino terminal end of the monomer.

In some embodiments, the inhibitor comprises any inhibitor capable of inhibiting, blocking, or otherwise inactivating one or more genes or gene products listed in Table 1, during prime editing with any one of the PE3, twinPE, or PrimeDel systems disclosed herein.

TABLE 1
Inhibition of genes (average of three sgRNAs per gene)
that enhance the editing efficiency of TwinPE editors
by a factor of greater than or equal to 1.3.

| | | | TwinPE |
|---|---|---|---|
| | Gene Name | | (approximate |
| ID No. | (total: 1329) | Unique Emsembl ID | fold-change) |
| 1 | CHAF1B | ENSG00000159259 | 3.6X |
| 2 | DBR1 | ENSG00000138231 | 3.2X |
| 3 | XRN2 | ENSG00000088930 | 2.6X |
| 4 | GTF2H4 | ENSG00000213780 | 2.6X |
| 5 | CCNH | ENSG00000134480 | 2.5X |
| 6 | MNAT1 | ENSG00000020426 | 2.5X |
| 7 | NOP10 | ENSG00000182117 | 2.4X |
| 8 | GTF2F2 | ENSG00000188342 | 2.3X |
| 9 | CDK7 | ENSG00000134058 | 2.3X |
| 10 | DROSHA | ENSG00000113360 | 2.3X |
| 11 | MYB | ENSG00000118513 | 2.2X |
| 12 | ERCC2 | ENSG00000104884 | 2.1X |
| 13 | HINFP | ENSG00000172273 | 2.1X |
| 14 | XRCC5 | ENSG00000079246 | 2.0X |
| 15 | CHAF1A | ENSG00000167670 | 2.0X |
| 16 | RPP40 | ENSG00000124787 | 2.0X |
| 17 | DKC1 | ENSG00000130826 | 2.0X |
| 18 | POP5 | ENSG00000167272 | 2.0X |
| 19 | TTI1 | ENSG00000101407 | 2.0X |
| 20 | RPP21 | ENSG00000241370 | 1.9X |
| 21 | TAF1 | ENSG00000147133 | 1.9X |
| 22 | HJURP | ENSG00000123485 | 1.9X |
| 23 | TSEN2 | ENSG00000154743 | 1.9X |
| 24 | DDX20 | ENSG00000064703 | 1.9X |
| 25 | GTF2H1 | ENSG00000110768 | 1.9X |
| 26 | SMARCA5 | ENSG00000153147 | 1.9X |
| 27 | URI1 | ENSG00000105176 | 1.9X |
| 28 | RPP30 | ENSG00000148688 | 1.8X |
| 29 | DICER1 | ENSG00000100697 | 1.8X |
| 30 | XRCC6 | ENSG00000196419 | 1.8X |
| 31 | GAR1 | ENSG00000109534 | 1.8X |
| 32 | MTOR | ENSG00000198793 | 1.8X |
| 33 | CENPP | ENSG00000188312 | 1.8X |
| 34 | EXOSC2 | ENSG00000130713 | 1.8X |
| 35 | HNRNPC | ENSG00000092199 | 1.8X |
| 36 | POP1 | ENSG00000104356 | 1.8X |
| 37 | INTS3 | ENSG00000143624 | 1.8X |
| 38 | NHP2 | ENSG00000145912 | 1.7X |
| 39 | ERCC3 | ENSG00000163161 | 1.7X |
| 40 | RAD17 | ENSG00000152942 | 1.7X |
| 41 | POLR2I | ENSG00000105258 | 1.7X |
| 42 | TELO2 | ENSG00000100726 | 1.7X |
| 43 | RPP14 | ENSG00000163684 | 1.7X |
| 44 | NSMCE1 | ENSG00000169189 | 1.7X |
| 45 | CDK12 | ENSG00000167258 | 1.7X |
| 46 | MIS18A | ENSG00000159055 | 1.7X |
| 47 | TTI2 | ENSG00000129696 | 1.7X |
| 48 | RCL1 | ENSG00000120158 | 1.7X |
| 49 | DDX5 | ENSG00000108654 | 1.6X |
| 50 | TREX2 | ENSG00000183479 | 1.6X |
| 51 | DIS3 | ENSG00000083520 | 1.6X |
| 52 | NAT10 | ENSG00000135372 | 1.6X |
| 53 | BCCIP | ENSG00000107949 | 1.6X |
| 54 | ELP3 | ENSG00000134014 | 1.6X |
| 55 | DHX36 | ENSG00000174953 | 1.6X |
| 56 | RPP38 | ENSG00000152464 | 1.6X |
| 57 | PPP1R8 | ENSG00000117751 | 1.6X |
| 58 | SLBP | ENSG00000163950 | 1.6X |
| 59 | EXOSC4 | ENSG00000178896 | 1.6X |
| 60 | CEP63 | ENSG00000182923 | 1.6X |
| 61 | BPTF | ENSG00000171634 | 1.6X |
| 62 | CPSF3 | ENSG00000119203 | 1.6X |
| 63 | LAS1L | ENSG00000001497 | 1.6X |
| 64 | SEM1 | ENSG00000127922 | 1.6X |
| 65 | GPN1 | ENSG00000198522 | 1.6X |
| 66 | TAF9 | ENSG00000273841 | 1.6X |
| 67 | SMC6 | ENSG00000163029 | 1.6X |
| 68 | MYBBP1A | ENSG00000132382 | 1.6X |
| 69 | POLR2H | ENSG00000163882 | 1.6X |
| 70 | HUWE1 | ENSG00000086758 | 1.6X |
| 71 | DDX27 | ENSG00000124228 | 1.6X |
| 72 | HUS1 | ENSG00000136273 | 1.6X |
| 73 | GRWD1 | ENSG00000105447 | 1.5X |
| 74 | CCNA2 | ENSG00000145386 | 1.5X |
| 75 | NSMCE4A | ENSG00000107672 | 1.5X |
| 76 | EXOSC5 | ENSG00000077348 | 1.5X |
| 77 | NCAPH | ENSG00000121152 | 1.5X |
| 78 | SMC2 | ENSG00000136824 | 1.5X |
| 79 | CENPA | ENSG00000115163 | 1.5X |
| 80 | MCM2 | ENSG00000073111 | 1.5X |
| 81 | TIMELESS | ENSG00000111602 | 1.5X |
| 82 | TFPT | ENSG00000105619 | 1.5X |
| 83 | HLTF | ENSG00000071794 | 1.5X |
| 84 | AATF | ENSG00000275700 | 1.5X |
| 85 | DDX21 | ENSG00000165732 | 1.5X |
| 86 | POLR2B | ENSG00000047315 | 1.5X |
| 87 | NONO | ENSG00000147140 | 1.5X |
| 88 | ASF1A | ENSG00000111875 | 1.5X |
| 89 | TOP1 | ENSG00000198900 | 1.5X |
| 90 | POLR2A | ENSG00000181222 | 1.5X |
| 91 | DHX35 | ENSG00000101452 | 1.5X |
| 92 | RAD9A | ENSG00000172613 | 1.5X |
| 93 | CHTF18 | ENSG00000127586 | 1.5X |
| 94 | POP7 | ENSG00000172336 | 1.5X |
| 95 | CENPW | ENSG00000203760 | 1.5X |
| 96 | APAF1 | ENSG00000120868 | 1.5X |
| 97 | RBMX | ENSG00000147274 | 1.5X |
| 98 | SMC5 | ENSG00000198887 | 1.5X |
| 99 | REXO2 | ENSG00000076043 | 1.5X |
| 100 | PRKDC | ENSG00000253729 | 1.5X |
| 101 | SART3 | ENSG00000075856 | 1.4X |
| 102 | GTF2H3 | ENSG00000111358 | 1.4X |
| 103 | EP400 | ENSG00000183495 | 1.4X |
| 104 | BRCA2 | ENSG00000139618 | 1.4X |
| 105 | MCM3 | ENSG00000112118 | 1.4X |
| 106 | NSMCE2 | ENSG00000156831 | 1.4X |
| 107 | SSRP1 | ENSG00000149136 | 1.4X |
| 108 | POLN | ENSG00000130997 | 1.4X |
| 109 | WAPL | ENSG00000062650 | 1.4X |
| 110 | MAU2 | ENSG00000129933 | 1.4X |
| 111 | TERF2 | ENSG00000132604 | 1.4X |
| 112 | ASCC3 | ENSG00000112249 | 1.4X |
| 113 | RAD51 | ENSG00000051180 | 1.4X |
| 114 | FIP1L1 | ENSG00000145216 | 1.4X |
| 115 | RAD1 | ENSG00000113456 | 1.4X |
| 116 | GTF3C4 | ENSG00000125484 | 1.4X |
| 117 | YEATS2 | ENSG00000163872 | 1.4X |
| 118 | NCAPG | ENSG00000109805 | 1.4X |
| 119 | MCM6 | ENSG00000076003 | 1.4X |
| 120 | PAXBP1 | ENSG00000159086 | 1.4X |
| 121 | DFFA | ENSG00000160049 | 1.4X |
| 122 | OGA | ENSG00000198408 | 1.4X |
| 123 | RBBP8 | ENSG00000101773 | 1.4X |
| 124 | POLR2G | ENSG00000168002 | 1.4X |
| 125 | POLR2L | ENSG00000177700 | 1.4X |
| 126 | SGO1 | ENSG00000129810 | 1.4X |
| 127 | DSCC1 | ENSG00000136982 | 1.4X |
| 128 | DDX49 | ENSG00000105671 | 1.4X |
| 129 | POLR2C | ENSG00000102978 | 1.4X |
| 130 | PRMT1 | ENSG00000126457 | 1.4X |
| 131 | SMC1A | ENSG00000072501 | 1.4X |
| 132 | NCAPD2 | ENSG00000010292 | 1.3X |
| 133 | BRD9 | ENSG00000028310 | 1.3X |
| 134 | BRD8 | ENSG00000112983 | 1.3X |
| 135 | RUVBL1 | ENSG00000175792 | 1.3X |
| 136 | RNF113A | ENSG00000125352 | 1.3X |
| 137 | DDX18 | ENSG00000088205 | 1.3X |
| 138 | PALB2 | ENSG00000083093 | 1.3X |
| 139 | UBE2S | ENSG00000108106 | 1.3X |
| 140 | DDX10 | ENSG00000178105 | 1.3X |
| 141 | DHX37 | ENSG00000150990 | 1.3X |
| 142 | CASP3 | ENSG00000164305 | 1.3X |
| 143 | ISG20L2 | ENSG00000143319 | 1.3X |
| 144 | MMS22L | ENSG00000146263 | 1.3X |
| 145 | DHX29 | ENSG00000067248 | 1.3X |
| 146 | DDX24 | ENSG00000089737 | 1.3X |
| 147 | COPS6 | ENSG00000168090 | 1.3X |
| 148 | PWP1 | ENSG00000136045 | 1.3X |
| 149 | SUPT16H | ENSG00000092201 | 1.3X |
| 150 | MRGBP | ENSG00000101189 | 1.3X |
| 151 | EXOSC3 | ENSG00000107371 | 1.3X |
| 152 | BUB1B | ENSG00000156970 | 1.3X |
| 153 | NOB1 | ENSG00000141101 | 1.3X |
| 154 | SAP18 | ENSG00000150459 | 1.3X |
| 155 | DDX51 | ENSG00000185163 | 1.3X |
| 156 | RNF8 | ENSG00000112130 | 1.3X |
| 157 | SUPT6H | ENSG00000109111 | 1.3X |
| 158 | SND1 | ENSG00000197157 | 1.3X |

In some embodiments, the inhibitor comprises any inhibitor capable of inhibiting, blocking, or otherwise inactivating one or more genes or gene products listed in Table 2, during prime editing with any one of the PrimeDel system disclosed herein.

TABLE 2
Inhibition of genes (average of three sgRNAs per gene)
that enhance the editing efficiency of PrimeDel editors
by a factor of greater than or equal to 1.3.

| | | | PrimeDEL |
|---|---|---|---|
| | Gene Name | | (approximate |
| ID No. | (total: 1329) | Unique Emsembl ID | fold-change) |
| 1 | CHAF1B | ENSG00000159259 | 2.3X |
| 2 | DBR1 | ENSG00000138231 | 1.9X |
| 3 | XRN2 | ENSG00000088930 | 2.7X |
| 4 | GTF2H4 | ENSG00000213780 | 1.6X |
| 5 | CCNH | ENSG00000134480 | 1.5X |
| 6 | MNAT1 | ENSG00000020426 | 1.4X |
| 7 | NOP10 | ENSG00000182117 | 1.5X |
| 8 | GTF2F2 | ENSG00000188342 | 1.5X |
| 9 | MYB | ENSG00000118513 | 1.4X |
| 10 | ERCC2 | ENSG00000104884 | 1.3X |
| 11 | HINFP | ENSG00000172273 | 1.5X |
| 12 | XRCC5 | ENSG00000079246 | 1.3X |
| 13 | CHAF1A | ENSG00000167670 | 1.7X |
| 14 | RPP40 | ENSG00000124787 | 1.4X |
| 15 | DKC1 | ENSG00000130826 | 1.3X |
| 16 | POP5 | ENSG00000167272 | 1.4X |
| 17 | RPP21 | ENSG00000241370 | 1.3X |
| 18 | TAF1 | ENSG00000147133 | 1.5X |
| 19 | HJURP | ENSG00000123485 | 1.4X |
| 20 | DDX20 | ENSG00000064703 | 1.4X |
| 21 | SMARCA5 | ENSG00000153147 | 1.6X |
| 22 | URI1 | ENSG00000105176 | 1.4X |
| 23 | RPP30 | ENSG00000148688 | 1.4X |
| 24 | GAR1 | ENSG00000109534 | 1.3X |
| 25 | EXOSC2 | ENSG00000130713 | 1.3X |
| 26 | INTS3 | ENSG00000143624 | 1.4X |
| 27 | RAD17 | ENSG00000152942 | 1.9X |
| 28 | NSMCE1 | ENSG00000169189 | 1.6X |
| 29 | CDK12 | ENSG00000167258 | 1.7X |
| 30 | MIS18A | ENSG00000159055 | 1.3X |
| 31 | RCL1 | ENSG00000120158 | 1.3X |
| 32 | TREX2 | ENSG00000183479 | 1.3X |
| 33 | DHX36 | ENSG00000174953 | 1.3X |
| 34 | PPP1R8 | ENSG00000117751 | 1.5X |
| 35 | SLBP | ENSG00000163950 | 1.6X |
| 36 | CPSF3 | ENSG00000119203 | 1.4X |
| 37 | SEM1 | ENSG00000127922 | 1.3X |
| 38 | TAF9 | ENSG00000273841 | 1.4X |
| 39 | SMC6 | ENSG00000163029 | 1.6X |
| 40 | POLR2H | ENSG00000163882 | 1.4X |
| 41 | HUS1 | ENSG00000136273 | 1.5X |
| 42 | CCNA2 | ENSG00000145386 | 1.4X |
| 43 | NSMCE4A | ENSG00000107672 | 1.5X |
| 44 | MCM2 | ENSG00000073111 | 1.6X |
| 45 | TIMELESS | ENSG00000111602 | 1.7X |
| 46 | HLTF | ENSG00000071794 | 1.5X |
| 47 | NONO | ENSG00000147140 | 1.5X |
| 48 | ASF1A | ENSG00000111875 | 1.3X |
| 49 | RAD9A | ENSG00000172613 | 1.6X |
| 50 | CHTF18 | ENSG00000127586 | 1.4X |
| 51 | RBMX | ENSG00000147274 | 1.3X |
| 52 | SMC5 | ENSG00000198887 | 1.6X |
| 53 | MCM3 | ENSG00000112118 | 1.4X |
| 54 | NSMCE2 | ENSG00000156831 | 1.4X |
| 55 | RAD51 | ENSG00000051180 | 1.5X |
| 56 | FIP1L1 | ENSG00000145216 | 1.4X |
| 57 | MCM6 | ENSG00000076003 | 1.5X |
| 58 | RBBP8 | ENSG00000101773 | 1.3X |
| 59 | MMS22L | ENSG00000146263 | 1.3X |
| 60 | MCM4 | ENSG00000104738 | 1.4X |
| 61 | MCM10 | ENSG00000065328 | 1.4X |
| 62 | DUT | ENSG00000128951 | 1.5X |
| 63 | SLX4 | ENSG00000188827 | 1.7X |
| 64 | SLF2 | ENSG00000119906 | 1.4X |
| 65 | FANCE | ENSG00000112039 | 1.7X |
| 66 | USP37 | ENSG00000135913 | 1.5X |
| 67 | RMI1 | ENSG00000178966 | 1.6X |
| 68 | SENP6 | ENSG00000112701 | 1.4X |
| 69 | RFWD3 | ENSG00000168411 | 1.6X |
| 70 | RBBP4 | ENSG00000162521 | 1.4X |
| 71 | ATRIP | ENSG00000164053 | 1.3X |
| 72 | TIPIN | ENSG00000075131 | 1.4X |
| 73 | FANCC | ENSG00000158169 | 1.4X |
| 74 | CFDP1 | ENSG00000153774 | 1.3X |
| 75 | ATRX | ENSG00000085224 | 1.3X |
| 76 | UBE2T | ENSG00000077152 | 2.0X |
| 77 | FANCI | ENSG00000140525 | 1.8X |
| 78 | FAAP24 | ENSG00000131944 | 1.6X |
| 79 | SRSF1 | ENSG00000136450 | 1.3X |
| 80 | HNRNPK | ENSG00000165119 | 1.4X |
| 81 | FANCD2 | ENSG00000144554 | 1.8X |
| 82 | FAAP100 | ENSG00000185504 | 1.3X |
| 83 | WDHD1 | ENSG00000198554 | 1.3X |
| 84 | CCAR1 | ENSG00000060339 | 1.3X |
| 85 | FANCM | ENSG00000187790 | 1.7X |
| 86 | FANCG | ENSG00000221829 | 1.5X |
| 87 | FANCL | ENSG00000115392 | 1.5X |
| 88 | FANCA | ENSG00000187741 | 1.6X |
| 89 | FANCB | ENSG00000181544 | 1.4X |
| 90 | UBR5 | ENSG00000104517 | 1.3X |
| 91 | TYMS | ENSG00000176890 | 1.3X |
| 92 | FANCF | ENSG00000183161 | 1.6X |

In some embodiments, the inhibitor comprises any inhibitor capable of inhibiting, blocking, or otherwise inactivating one or more genes or gene products listed in Table 3, during prime editing with any one of the PE3 systems disclosed herein.

TABLE 3
Inhibition of genes (average of three sgRNAs per gene)
that enhance the editing efficiency of PE3 editors
by a factor of greater than or equal to 1.3.

| | | | PE3 |
|---|---|---|---|
| | Gene Name | | (approximate |
| ID No. | (total: 1329) | Unique Emsembl ID | fold-change) |
| 1 | CHAF1B | ENSG00000159259 | 1.6X |
| 2 | DBR1 | ENSG00000138231 | 2.9X |
| 3 | XRN2 | ENSG00000088930 | 2.4X |
| 4 | CCNH | ENSG00000134480 | 1.3X |
| 5 | NOP10 | ENSG00000182117 | 1.9X |
| 6 | MYB | ENSG00000118513 | 1.4X |
| 7 | XRCC5 | ENSG00000079246 | 1.6X |
| 8 | DKC1 | ENSG00000130826 | 1.7X |
| 9 | TAF1 | ENSG00000147133 | 1.7X |
| 10 | TSEN2 | ENSG00000154743 | 1.4X |
| 11 | SMARCA5 | ENSG00000153147 | 1.8X |
| 12 | XRCC6 | ENSG00000196419 | 1.6X |
| 13 | GAR1 | ENSG00000109534 | 1.6X |
| 14 | CENPP | ENSG00000188312 | 1.5X |
| 15 | EXOSC2 | ENSG00000130713 | 2.2X |
| 16 | HNRNPC | ENSG00000092199 | 1.4X |
| 17 | NHP2 | ENSG00000145912 | 1.4X |
| 18 | NSMCE1 | ENSG00000169189 | 2.0X |
| 19 | CDK12 | ENSG00000167258 | 1.4X |
| 20 | RCL1 | ENSG00000120158 | 1.6X |
| 21 | DIS3 | ENSG00000083520 | 1.8X |
| 22 | NAT10 | ENSG00000135372 | 1.5X |
| 23 | DHX36 | ENSG00000174953 | 1.3X |
| 24 | PPP1R8 | ENSG00000117751 | 1.4X |
| 25 | EXOSC4 | ENSG00000178896 | 2.1X |
| 26 | BPTF | ENSG00000171634 | 1.5X |
| 27 | LAS1L | ENSG00000001497 | 1.4X |
| 28 | SMC6 | ENSG00000163029 | 1.9X |
| 29 | MYBBP1A | ENSG00000132382 | 1.4X |
| 30 | DDX27 | ENSG00000124228 | 1.5X |
| 31 | NSMCE4A | ENSG00000107672 | 1.7X |
| 32 | EXOSC5 | ENSG00000077348 | 2.1X |
| 33 | MCM2 | ENSG00000073111 | 1.4X |
| 34 | AATF | ENSG00000275700 | 1.5X |
| 35 | DDX21 | ENSG00000165732 | 1.3X |
| 36 | NONO | ENSG00000147140 | 1.5X |
| 37 | ASF1A | ENSG00000111875 | 1.4X |
| 38 | TOP1 | ENSG00000198900 | 1.3X |
| 39 | SMC5 | ENSG00000198887 | 1.9X |
| 40 | NSMCE2 | ENSG00000156831 | 1.7X |
| 41 | WAPL | ENSG00000062650 | 1.3X |
| 42 | RAD51 | ENSG00000051180 | 1.4X |
| 43 | FIP1L1 | ENSG00000145216 | 1.4X |
| 44 | GTF3C4 | ENSG00000125484 | 1.6X |
| 45 | MCM6 | ENSG00000076003 | 1.3X |
| 46 | SMC1A | ENSG00000072501 | 1.6X |
| 47 | DDX18 | ENSG00000088205 | 1.3X |
| 48 | DHX37 | ENSG00000150990 | 1.4X |
| 49 | DDX24 | ENSG00000089737 | 1.5X |
| 50 | EXOSC3 | ENSG00000107371 | 1.8X |
| 51 | SAP18 | ENSG00000150459 | 1.4X |
| 52 | DDX51 | ENSG00000185163 | 1.4X |
| 53 | DDX56 | ENSG00000136271 | 1.3X |
| 54 | DUT | ENSG00000128951 | 1.3X |
| 55 | CABIN1 | ENSG00000099991 | 1.4X |
| 56 | SLF2 | ENSG00000119906 | 1.6X |
| 57 | HDAC3 | ENSG00000171720 | 1.3X |
| 58 | DDX47 | ENSG00000213782 | 1.3X |
| 59 | RNF4 | ENSG00000063978 | 1.3X |
| 60 | EXOSC9 | ENSG00000123737 | 1.3X |
| 61 | MTREX | ENSG00000039123 | 1.4X |
| 62 | CNOT4 | ENSG00000080802 | 1.3X |
| 63 | EXOSC7 | ENSG00000075914 | 1.3X |
| 64 | ATRX | ENSG00000085224 | 1.4X |
| 65 | SAE1 | ENSG00000142230 | 1.3X |
| 66 | NELFB | ENSG00000188986 | 1.3X |
| 67 | PNPT1 | ENSG00000138035 | 1.3X |
| 68 | MNAT1 | ENSG00000020426 | 1.3X |

In some embodiments, the inhibitor comprises any inhibitor capable of inhibiting, blocking, or otherwise inactivating one or more genes or gene products listed in Table 4, during prime editing with any one of the twinPE or PrimeDel systems disclosed herein.

TABLE 4
Inhibition of genes (average of three sgRNAs per gene)
that enhance the editing efficiency of TwinPE and PrimeDel
editors by a factor of greater than or equal to 1.3.

| | | | TwinPE | PrimeDEL |
|---|---|---|---|---|
| | Gene Name | Unique | (approximate | (approximate |
| ID No. | (total: 1329) | Emsembl ID | fold-change) | fold-change) |
| 1 | CHAF1B | ENSG00000159259 | 3.6X | 2.3X |
| 2 | DBR1 | ENSG00000138231 | 3.2X | 1.9X |
| 3 | XRN2 | ENSG00000088930 | 2.6X | 2.7X |
| 4 | GTF2H4 | ENSG00000213780 | 2.6X | 1.6X |
| 5 | CCNH | ENSG00000134480 | 2.5X | 1.5X |
| 6 | MNAT1 | ENSG00000020426 | 2.5X | 1.4X |
| 7 | NOP10 | ENSG00000182117 | 2.4X | 1.5X |
| 8 | GTF2F2 | ENSG00000188342 | 2.3X | 1.5X |
| 9 | MYB | ENSG00000118513 | 2.2X | 1.4X |
| 10 | ERCC2 | ENSG00000104884 | 2.1X | 1.3X |
| 11 | HINFP | ENSG00000172273 | 2.1X | 1.5X |
| 12 | XRCC5 | ENSG00000079246 | 2.0X | 1.3X |
| 13 | CHAF1A | ENSG00000167670 | 2.0X | 1.7X |
| 14 | RPP40 | ENSG00000124787 | 2.0X | 1.4X |
| 15 | DKC1 | ENSG00000130826 | 2.0X | 1.3X |
| 16 | POP5 | ENSG00000167272 | 2.0X | 1.4X |
| 17 | RPP21 | ENSG00000241370 | 1.9X | 1.3X |
| 18 | TAF1 | ENSG00000147133 | 1.9X | 1.5X |
| 19 | HJURP | ENSG00000123485 | 1.9X | 1.4X |
| 20 | DDX20 | ENSG00000064703 | 1.9X | 1.4X |
| 21 | SMARCA5 | ENSG00000153147 | 1.9X | 1.6X |
| 22 | URI1 | ENSG00000105176 | 1.9X | 1.4X |
| 23 | RPP30 | ENSG00000148688 | 1.8X | 1.4X |
| 24 | GAR1 | ENSG00000109534 | 1.8X | 1.3X |
| 25 | EXOSC2 | ENSG00000130713 | 1.8X | 1.3X |
| 26 | INTS3 | ENSG00000143624 | 1.8X | 1.4X |
| 27 | RAD17 | ENSG00000152942 | 1.7X | 1.9X |
| 28 | NSMCE1 | ENSG00000169189 | 1.7X | 1.6X |
| 29 | CDK12 | ENSG00000167258 | 1.7X | 1.7X |
| 30 | MIS18A | ENSG00000159055 | 1.7X | 1.3X |
| 31 | RCL1 | ENSG00000120158 | 1.7X | 1.3X |
| 32 | TREX2 | ENSG00000183479 | 1.6X | 1.3X |
| 33 | DHX36 | ENSG00000174953 | 1.6X | 1.3X |
| 34 | PPP1R8 | ENSG00000117751 | 1.6X | 1.5X |
| 35 | SLBP | ENSG00000163950 | 1.6X | 1.6X |
| 36 | CPSF3 | ENSG00000119203 | 1.6X | 1.4X |
| 37 | SEM1 | ENSG00000127922 | 1.6X | 1.3X |
| 38 | TAF9 | ENSG00000273841 | 1.6X | 1.4X |
| 39 | SMC6 | ENSG00000163029 | 1.6X | 1.6X |
| 40 | POLR2H | ENSG00000163882 | 1.6X | 1.4X |
| 41 | HUS1 | ENSG00000136273 | 1.6X | 1.5X |
| 42 | CCNA2 | ENSG00000145386 | 1.5X | 1.4X |
| 43 | NSMCE4A | ENSG00000107672 | 1.5X | 1.5X |
| 44 | MCM2 | ENSG00000073111 | 1.5X | 1.6X |
| 45 | TIMELESS | ENSG00000111602 | 1.5X | 1.7X |
| 46 | HLTF | ENSG00000071794 | 1.5X | 1.5X |
| 47 | NONO | ENSG00000147140 | 1.5X | 1.5X |
| 48 | ASF1A | ENSG00000111875 | 1.5X | 1.3X |
| 49 | RAD9A | ENSG00000172613 | 1.5X | 1.6X |
| 50 | CHTF18 | ENSG00000127586 | 1.5X | 1.4X |
| 51 | RBMX | ENSG00000147274 | 1.5X | 1.3X |
| 52 | SMC5 | ENSG00000198887 | 1.5X | 1.6X |
| 53 | MCM3 | ENSG00000112118 | 1.4X | 1.4X |
| 54 | NSMCE2 | ENSG00000156831 | 1.4X | 1.4X |
| 55 | RAD51 | ENSG00000051180 | 1.4X | 1.5X |
| 56 | FIP1L1 | ENSG00000145216 | 1.4X | 1.4X |
| 57 | MCM6 | ENSG00000076003 | 1.4X | 1.5X |
| 58 | RBBP8 | ENSG00000101773 | 1.4X | 1.3X |
| 59 | MMS22L | ENSG00000146263 | 1.3X | 1.3X |

In some embodiments, the inhibitor comprises any inhibitor capable of inhibiting, blocking, or otherwise inactivating one or more genes or gene products listed in Table 5, during prime editing with any one of the twinPE or PE3 systems disclosed herein.

TABLE 5
Inhibition of genes (average of three sgRNAs per gene)
that enhance the editing efficiency of TwinPE and PE3
editors by a factor of greater than or equal to 1.3.

| | | | TwinPE | PE3 |
|---|---|---|---|---|
| | Gene Name | | (approximate | (approximate |
| ID No. | (total: 1329) | Unique Emsembl ID | fold-change) | fold-change) |
| 1 | CHAF1B | ENSG00000159259 | 3.6X | 1.6X |
| 2 | DBR1 | ENSG00000138231 | 3.2X | 2.9X |
| 3 | XRN2 | ENSG00000088930 | 2.6X | 2.4X |
| 4 | CCNH | ENSG00000134480 | 2.5X | 1.3X |
| 5 | NOP10 | ENSG00000182117 | 2.4X | 1.9X |
| 6 | MYB | ENSG00000118513 | 2.2X | 1.4X |
| 7 | XRCC5 | ENSG00000079246 | 2.0X | 1.6X |
| 8 | DKC1 | ENSG00000130826 | 2.0X | 1.7X |
| 9 | TAF1 | ENSG00000147133 | 1.9X | 1.7X |
| 10 | TSEN2 | ENSG00000154743 | 1.9X | 1.4X |
| 11 | SMARCA5 | ENSG00000153147 | 1.9X | 1.8X |
| 12 | XRCC6 | ENSG00000196419 | 1.8X | 1.6X |
| 13 | GAR1 | ENSG00000109534 | 1.8X | 1.6X |
| 14 | CENPP | ENSG00000188312 | 1.8X | 1.5X |
| 15 | EXOSC2 | ENSG00000130713 | 1.8X | 2.2X |
| 16 | HNRNPC | ENSG00000092199 | 1.8X | 1.4X |
| 17 | NHP2 | ENSG00000145912 | 1.7X | 1.4X |
| 18 | NSMCE1 | ENSG00000169189 | 1.7X | 2.0X |
| 19 | CDK12 | ENSG00000167258 | 1.7X | 1.4X |
| 20 | RCL1 | ENSG00000120158 | 1.7X | 1.6X |
| 21 | DIS3 | ENSG00000083520 | 1.6X | 1.8X |
| 22 | NAT10 | ENSG00000135372 | 1.6X | 1.5X |
| 23 | DHX36 | ENSG00000174953 | 1.6X | 1.3X |
| 24 | PPP1R8 | ENSG00000117751 | 1.6X | 1.4X |
| 25 | EXOSC4 | ENSG00000178896 | 1.6X | 2.1X |
| 26 | BPTF | ENSG00000171634 | 1.6X | 1.5X |
| 27 | LAS1L | ENSG00000001497 | 1.6X | 1.4X |
| 28 | SMC6 | ENSG00000163029 | 1.6X | 1.9X |
| 29 | MYBBP1A | ENSG00000132382 | 1.6X | 1.4X |
| 30 | DDX27 | ENSG00000124228 | 1.6X | 1.5X |
| 31 | NSMCE4A | ENSG00000107672 | 1.5X | 1.7X |
| 32 | EXOSC5 | ENSG00000077348 | 1.5X | 2.1X |
| 33 | MCM2 | ENSG00000073111 | 1.5X | 1.4X |
| 34 | AATF | ENSG00000275700 | 1.5X | 1.5X |
| 35 | DDX21 | ENSG00000165732 | 1.5X | 1.3X |
| 36 | NONO | ENSG00000147140 | 1.5X | 1.5X |
| 37 | ASF1A | ENSG00000111875 | 1.5X | 1.4X |
| 38 | TOP1 | ENSG00000198900 | 1.5X | 1.3X |
| 39 | SMC5 | ENSG00000198887 | 1.5X | 1.9X |
| 40 | NSMCE2 | ENSG00000156831 | 1.4X | 1.7X |
| 41 | WAPL | ENSG00000062650 | 1.4X | 1.3X |
| 42 | RAD51 | ENSG00000051180 | 1.4X | 1.4X |
| 43 | FIP1L1 | ENSG00000145216 | 1.4X | 1.4X |
| 44 | GTF3C4 | ENSG00000125484 | 1.4X | 1.6X |
| 45 | MCM6 | ENSG00000076003 | 1.4X | 1.3X |
| 46 | SMC1A | ENSG00000072501 | 1.4X | 1.6X |
| 47 | DDX18 | ENSG00000088205 | 1.3X | 1.3X |
| 48 | DHX37 | ENSG00000150990 | 1.3X | 1.4X |
| 49 | DDX24 | ENSG00000089737 | 1.3X | 1.5X |
| 50 | EXOSC3 | ENSG00000107371 | 1.3X | 1.8X |
| 51 | SAP18 | ENSG00000150459 | 1.3X | 1.4X |
| 52 | DDX51 | ENSG00000185163 | 1.3X | 1.4X |

In some embodiments, the inhibitor comprises any inhibitor capable of inhibiting, blocking, or otherwise inactivating one or more genes or gene products listed in Table 6, during prime editing with any one of the PrimeDel or PE3 systems disclosed herein.

TABLE 6
Inhibition of genes (average of three sgRNAs per gene)
that enhance the editing efficiency of PrimeDel and PE3
editors by a factor of greater than or equal to 1.3.

| | | | PrimeDEL | PE3 |
|---|---|---|---|---|
| | Gene Name | | (approximate | (approximate |
| ID No. | (total: 1329) | Unique Emsembl ID | fold-change) | fold-change) |
| 1 | CHAF1B | ENSG00000159259 | 2.3X | 1.6X |
| 2 | DBR1 | ENSG00000138231 | 1.9X | 2.9X |
| 3 | XRN2 | ENSG00000088930 | 2.7X | 2.4X |
| 4 | CCNH | ENSG00000134480 | 1.5X | 1.3X |
| 5 | NOP10 | ENSG00000182117 | 1.5X | 1.9X |
| 6 | MYB | ENSG00000118513 | 1.4X | 1.4X |
| 7 | XRCC5 | ENSG00000079246 | 1.3X | 1.6X |
| 8 | DKC1 | ENSG00000130826 | 1.3X | 1.7X |
| 9 | TAF1 | ENSG00000147133 | 1.5X | 1.7X |
| 10 | SMARCA5 | ENSG00000153147 | 1.6X | 1.8X |
| 11 | GAR1 | ENSG00000109534 | 1.3X | 1.6X |
| 12 | EXOSC2 | ENSG00000130713 | 1.3X | 2.2X |
| 13 | NSMCE1 | ENSG00000169189 | 1.6X | 2.0X |
| 14 | CDK12 | ENSG00000167258 | 1.7X | 1.4X |
| 15 | RCL1 | ENSG00000120158 | 1.3X | 1.6X |
| 16 | DHX36 | ENSG00000174953 | 1.3X | 1.3X |
| 17 | PPP1R8 | ENSG00000117751 | 1.5X | 1.4X |
| 18 | SMC6 | ENSG00000163029 | 1.6X | 1.9X |
| 19 | NSMCE4A | ENSG00000107672 | 1.5X | 1.7X |
| 20 | MCM2 | ENSG00000073111 | 1.6X | 1.4X |
| 21 | NONO | ENSG00000147140 | 1.5X | 1.5X |
| 22 | ASF1A | ENSG00000111875 | 1.3X | 1.4X |
| 23 | SMC5 | ENSG00000198887 | 1.6X | 1.9X |
| 24 | NSMCE2 | ENSG00000156831 | 1.4X | 1.7X |
| 25 | RAD51 | ENSG00000051180 | 1.5X | 1.4X |
| 26 | FIP1L1 | ENSG00000145216 | 1.4X | 1.4X |
| 27 | MCM6 | ENSG00000076003 | 1.5X | 1.3X |
| 28 | DUT | ENSG00000128951 | 1.5X | 1.3X |
| 29 | SLF2 | ENSG00000119906 | 1.4X | 1.6X |
| 30 | ATRX | ENSG00000085224 | 1.3X | 1.4X |

In some embodiments, the inhibitor comprises any inhibitor capable of inhibiting, blocking, or otherwise inactivating one or more genes or gene products listed in Table 6, during prime editing with any one of the twinPE, PrimeDel or PE3 systems disclosed herein.

TABLE 7
Inhibition of genes (average of three sgRNAs per gene) that
enhance the editing efficiency of TwinPE, PrimeDel, and
PE3 editors by a factor of greater than or equal to 1.3.

| | | | TwinPE | PrimeDEL | PE3 |
|---|---|---|---|---|---|
| | Gene Name | Unique Emsembl | (approximate | (approximate | (approximate |
| ID No. | (total: 1329) | ID | fold-change) | fold-change) | fold-change) |
| 1 | CHAF1B | ENSG00000159259 | 3.6X | 2.3X | 1.6X |
| 2 | DBR1 | ENSG00000138231 | 3.2X | 1.9X | 2.9X |
| 3 | XRN2 | ENSG00000088930 | 2.6X | 2.7X | 2.4X |
| 4 | CCNH | ENSG00000134480 | 2.5X | 1.5X | 1.3X |
| 5 | NOP10 | ENSG00000182117 | 2.4X | 1.5X | 1.9X |
| 6 | MYB | ENSG00000118513 | 2.2X | 1.4X | 1.4X |
| 7 | XRCC5 | ENSG00000079246 | 2.0X | 1.3X | 1.6X |
| 8 | DKC1 | ENSG00000130826 | 2.0X | 1.3X | 1.7X |
| 9 | TAF1 | ENSG00000147133 | 1.9X | 1.5X | 1.7X |
| 10 | SMARCA5 | ENSG00000153147 | 1.9X | 1.6X | 1.8X |
| 11 | GAR1 | ENSG00000109534 | 1.8X | 1.3X | 1.6X |
| 12 | EXOSC2 | ENSG00000130713 | 1.8X | 1.3X | 2.2X |
| 13 | NSMCE1 | ENSG00000169189 | 1.7X | 1.6X | 2.0X |
| 14 | CDK12 | ENSG00000167258 | 1.7X | 1.7X | 1.4X |
| 15 | RCL1 | ENSG00000120158 | 1.7X | 1.3X | 1.6X |
| 16 | DHX36 | ENSG00000174953 | 1.6X | 1.3X | 1.3X |
| 17 | PPP1R8 | ENSG00000117751 | 1.6X | 1.5X | 1.4X |
| 18 | SMC6 | ENSG00000163029 | 1.6X | 1.6X | 1.9X |
| 19 | NSMCE4A | ENSG00000107672 | 1.5X | 1.5X | 1.7X |
| 20 | MCM2 | ENSG00000073111 | 1.5X | 1.6X | 1.4X |
| 21 | NONO | ENSG00000147140 | 1.5X | 1.5X | 1.5X |
| 22 | ASF1A | ENSG00000111875 | 1.5X | 1.3X | 1.4X |
| 23 | SMC5 | ENSG00000198887 | 1.5X | 1.6X | 1.9X |
| 24 | NSMCE2 | ENSG00000156831 | 1.4X | 1.4X | 1.7X |
| 25 | RAD51 | ENSG00000051180 | 1.4X | 1.5X | 1.4X |
| 26 | FIP1L1 | ENSG00000145216 | 1.4X | 1.4X | 1.4X |
| 27 | MCM6 | ENSG00000076003 | 1.4X | 1.5X | 1.3X |

Likewise, it has also been discovered that certain genes and gene products involved in MMR, chromatin/chromatin-binding, chromatin remodeling, nuclease activity, helicase activity, and DNA damage response, which, when knocked down or knocked out, decrease the editing efficiency of the PE3, twinPE, and PrimeDel prime editing systems (e.g., they act to enhanced prime editing). Thus, various aspects of the present disclosure relate to stimulating, enhancing, or otherwise activating one or more of the genes associated with chromatin/chromatin-binding, chromatin remodeling, nuclease activity, helicase activity, and DNA damage response as listed in Tables X4, X6, and X8 (e.g., in addition to the MMR pathway). In some embodiments, the one or more activators of the one or more genes or gene products listed in Tables X4, X6, and X8 do not include the MMR pathway.

Any suitable method of stimulating, enhancing, or otherwise activating one or more of the genes listed in Tables X4, X6, and X8, known to the skilled artisan, is herein contemplated. In some embodiments, gene activation involves the use of one or more inhibitors to regulate transcriptional control of the gene, translational control of the gene, and/or posttranslational control of the gene. Alternatively, or additionally, gene activation involves the use of one or more nucleic acid molecules encoding the one or more genes or gene products listed in Tables X4, X6, and X8. In addition, the disclosure contemplates that the prime editor (e.g., delivered as a fusion protein comprising a napDNAbp and a polymerase, such as a Cas9 nickase fused to a reverse transcriptase) may be administered together with a gene enhancer.

In some embodiments, the enhancer is a messenger RNA. In other embodiments, the enhancer is a DNA molecule. In some embodiments, the enhancer is encoded within an AAV vector configured to deliver the enhancer to one or more cells, in vitro or in vivo.

In some embodiments, a cell may be engineered to overexpress one or more of the genes listed in Tables X4, X6, and X8. In some embodiments, the cell is a mammalian cells or a plant cell. In some cases, the mammalian cell is a human cell.

In some embodiments, the enhancer comprises any agent capable of stimulating, enhancing, or otherwise activating one or more of the genes associated with chromatin/chromatin-binding, chromatin remodeling, nuclease activity, helicase activity, and DNA damage response as listed in Tables X4, X6, and X8, during prime editing with any one of the prime editing systems disclosed herein.

### Prime Editors

The present disclosure contemplates using prime editors comprising fusion proteins, wherein the fusion proteins comprise a nucleic acid programmable DNA binding protein (napDNAbp) domain and a polymerase (e.g., reverse transcriptase) domain. Any suitable napDNAbp and polymerase known in the art may be combined into a single fusion protein with any suitable structural configuration, in accordance with some embodiments. For example, the fusion protein may comprise, from the N-terminus to the C-terminus direction, a napDNAbp fused to a polymerase. In other embodiments, the fusion protein may comprise from the N-terminus to the C-terminus direction, a polymerase fused to a napDNAbp. The fused domain may optionally be joined by a linker, e.g., an amino acid sequence. In other embodiments, the fusion proteins may comprise the structure NH₂-[napDNAbp]-[polymerase]-COOH; or NH₂-[polymerase]-[napDNAbp]-COOH, wherein each instance of “]-[” indicates the presence of an optional linker sequence. In embodiments wherein the polymerase is a reverse transcriptase, the fusion proteins may comprise the structure NH₂-[napDNAbp]-[RT]-COOH; or NH₂-[RT]-[napDNAbp]-COOH, wherein each instance of “]-[” indicates the presence of an optional linker sequence.

Since prime editors, and hence napDNAbps and polymerases, are well-known in the art, and the amino acid sequences are readily available, this disclosure is not meant in any way to be limited to those specific napDNAbps and/or polymerases identified herein. Non-limiting examples of prime editors contemplated herein may be found in U.S. Provisional Application No. 62/820,813, U.S. Provisional Application No. 62/858,958, U.S. Provisional Application No. 62/889,996, U.S. Provisional Application No. 62/922,654, U.S. Provisional Application No. 62/913,553, U.S. Provisional Application No. 62/973,558, U.S. Provisional Application No. 62/931,195, U.S. Provisional Application No. 62/944,231, U.S. Provisional Application No. 62/974,537, U.S. Provisional Application No. 62/991,069, U.S. Provisional Application No. 63/100,548, U.S. Provisional Application No. 63/022,397, U.S. Provisional Application No. 63/116,785, International PCT Application No. PCT/US2020/023721, International PCT Application No. PCT/US2020/023553, International PCT Application No. PCT/US2020/023583, International PCT Application No. PCT/US2020/023730, International PCT Application No. PCT/US2020/023713, International PCT Application No. PCT/US2020/023712, International PCT Application No. PCT/US2020/023727, International PCT Application No. PCT/US2020/023724, International PCT Application No. PCT/US2020/023725, International PCT Application No. PCT/US2020/023728, International PCT Application No. PCT/US2020/023732, International PCT Application No. PCT/US2020/023723, and International PCT Application No. PCT/US2021/031439, all of which are herein incorporated by reference in their entirety.

In some embodiments, the napDNAbp domain and the polymerase domain are fused together without a linker. In other embodiments, the napDNAbp domain is fused to the polymerase domain via a linker. Any suitable linker known in the art may be used to fuse the napDNAbp domain and the polymerase domain. For example, in some embodiments, the linker is a peptide, a polypeptide, a protein, a nucleic acid, a polymer, a polysaccharide, or any combination thereof.

In some embodiments, the fusion proteins may comprise any suitable structural configuration. For example, the fusion protein may comprise from the N-terminus to the C-terminus direction, a napDNAbp fused to a polymerase (e.g., DNA-dependent DNA polymerase or RNA-dependent DNA polymerase, such as, reverse transcriptase). In other embodiments, the fusion protein may comprise from the N-terminus to the C-terminus direction, a polymerase (e.g., a reverse transcriptase) fused to a napDNAbp. The fused domain may optionally be joined by a linker, e.g., an amino acid sequence. In other embodiments, the fusion proteins may comprise the structure NH₂-[napDNAbp]-[polymerase]-COOH; or NH₂-[polymerase]-[napDNAbp]-COOH, wherein each instance of “]-[” indicates the presence of an optional linker sequence. In embodiments wherein the polymerase is a reverse transcriptase, the fusion proteins may comprise the structure NH₂-[napDNAbp]-[RT]-COOH; or NH₂-[RT]-[napDNAbp]-COOH, wherein each instance of “]-[” indicates the presence of an optional linker sequence.

In various embodiments, the prime editor fusion protein may have the following structure (referred to herein as “PE1”), which includes a Cas9 variant comprising an H840A mutation (i.e., a Cas9 nickase) and an M-MLV RT wild type, as well as an N-terminal NLS sequence (19 amino acids) and an amino acid linker (32 amino acids) that joins the C-terminus of the Cas9 nickase domain to the N-terminus of the RT domain. The PE1 fusion protein has the following structure: [NLS]-[Cas9 (H840A)]-[linker]-[MMLV_RT (wt)].

In some embodiments, the prime editor fusion protein (referred to herein as “PE2”) comprises a Cas9 (H840A) and a variant MMLV RT having the following structure: [NLS]-[Cas9 (H840A)]-[linker]-[MMLV_RT (D200N) (T330P) (L603W) (T306K) (W313F)] and a desired pegRNA.

In one set of embodiments, a prime editor of the present disclosure is selected from the group consisting of PE3 editors, TwinPE editors, and Prime Del editors as described in Anzalone et al., “Search-and-replace genome editing without double-strand breaks or donor DNA,” Nature. 2019 December; 576 (7785): 149-157; Anzalone et al., “Programmable deletion, replacement, integration, and inversion of large DNA sequences with twin prime editing,” Nat. Biotechnol. 2022 May; 40 (5): 731-740; and by Choi et al., “Precise genomic deletions using paired prime editing,” Nat. Biotechnol. 2022 February; 40 (2): 218-226, all of which are herein incorporated by reference in their entirety. In some embodiments, TwinPE and Prime Del editors comprise a pair of PE2 editors and two pegRNAs that target opposite strands of a double stranded nucleic acid (e.g., DNA).

In some embodiments, a PE3 prime editor comprises PE2 machinery and an additional sgRNA.

In some embodiments, a TwinPE editor comprises a first prime editor and a second prime editor. In some embodiments, the first prime editor comprises a first prime editor comprising a first nucleic acid programmable DNA binding protein (first napDNAbp) and a first polypeptide comprising an RNA-dependent DNA polymerase activity. The first prime editor further comprises a first prime editing guide RNA (first pegRNA) that binds to a first binding site on a first strand of the double-stranded DNA sequence upstream of the target site to be edited. In some embodiments, the first prime editor is a first PE2 editor. In some embodiments, the second prime editor comprises a second prime editor comprising a second nucleic acid programmable DNA binding protein (second napDNAbp) and a second polypeptide comprising an RNA-dependent DNA polymerase activity. The second prime editor further comprises a second prime editing guide RNA (second pegRNA) that binds to a second binding site on a second strand of the double-stranded DNA sequence upstream of the target site to be edited. In some embodiments, the second prime editor is a second PE2 editor. In some embodiments, the first pegRNA comprises a first DNA synthesis template encoding a first single-stranded DNA sequence and the second pegRNA comprises a second DNA synthesis template encoding a second single-stranded DNA sequence. In some embodiments, the first and the second single-stranded DNA sequence each comprise a region of complementarity to the other. In some embodiments, the first single-stranded DNA sequence and the second single-stranded DNA sequence form a duplex comprising an edited portion as compared to the DNA sequence at the target site to be edited.

In some embodiments, a Prime Del prime editor comprises a first prime editor and a second prime editor. In some embodiments, the first prime editor comprises a first prime editor comprising a first nucleic acid programmable DNA binding protein (first napDNAbp) and a first polypeptide comprising an RNA-dependent DNA polymerase activity. The first prime editor further comprises a first prime editing guide RNA (first pegRNA) that binds to a first binding site on a first strand of the double-stranded DNA sequence upstream of the target site to be edited. In some embodiments, the second prime editor comprises a second nucleic acid programmable DNA binding protein (second napDNAbp) and a second polypeptide comprising an RNA-dependent DNA polymerase activity. The second prime editor further comprises a second prime editing guide RNA (second pegRNA) that binds to a second binding site on a second strand of the double-stranded DNA sequence upstream of the target site to be edited. In some embodiments, the first pegRNA comprises a first DNA synthesis template that comprises a region of complementary to the second binding site of the second pegRNA. In some embodiments, the second pegRNA comprises a second DNA synthesis template that comprises a region of complementary to the first binding site of the first pegRNA. In some embodiments, the first DNA synthesis template is used to produce a first single stranded DNA sequence and the second DNA synthesis template is used to produce a second single stranded DNA sequence. In some embodiments, first single stranded DNA sequence forms a duplex with a sequence complementary to the binding site of the second pegRNA and the second single stranded DNA sequence forms a duplex with a sequence complementary to the binding site of the first pegRNA.

napDNAbp Domain

In some embodiments, a prime editor comprises a (napDNAbp) domain. Any suitable napDNAbp domain known in the art may be used in the prime editors described herein, such as those described in detail in U.S. Patent Application 63/136,194, titled “Prime editor variants, constructs, and methods of using the same” by David Liu, et al., filed on Jan. 11, 2021, which is incorporated herein by reference in its entirety. For example, in various embodiments, the napDNAbp may be any Class 2 CRISPR-Cas system, including any type II, type V, or type VI CRISPR-Cas enzyme. Given the rapid development of CRISPR-Cas as a tool for genome editing, there have been constant developments in the nomenclature used to describe and/or identify CRISPR-Cas enzymes, such as Cas9 and Cas9 orthologs. This application references CRISPR-Cas enzymes with nomenclature that may be old and/or new as described in U.S. Patent Application 63/136,194 (described elsewhere herein) or Makarova et al., The CRISPR Journal, Vol. 1, No. 5, 2018, which is incorporated herein by reference in its entirety.

Other napDNAbps are also possible in other embodiments. For example, in some embodiments, the napDNAbp comprises the canonical SpCas9, or any ortholog Cas9 protein, or any variant Cas9 protein—including any naturally occurring variant, mutant, or otherwise engineered version of Cas9—that is known or that may be made or evolved through a directed evolutionary or otherwise mutagenic process. In various embodiments, the Cas9 or Cas9 variants have a nickase activity, i.e., only cleave one strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats).

In various embodiments described herein, the prime editors comprise a napDNAbp, such as a Cas9 protein. These proteins are “programmable” by way of their becoming complexed with a guide RNA (or a pegRNA, as the case may be), which guides the Cas9 protein to a target site on the DNA which possess a sequence that is complementary to the spacer portion of the gRNA (or pegRNA) and also which possesses the required PAM sequence. However, in certain embodiment envisioned here, the napDNAbp may be substituted with a different type of programmable protein, such as a zinc finger nuclease or a transcription activator-like effector nuclease (TALEN). See U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety. In addition, TALENS are described in WO 2015/027134, U.S. Pat. No. 9,181,535, Boch et al., “Breaking the Code of DNA Binding Specificity of TAL-Type III Effectors”, Science, vol. 326, pp. 1509-1512 (2009), Bogdanove et al., TAL Effectors: Customizable Proteins for DNA Targeting, Science, vol. 333, pp. 1843-1846 (2011), Cade et al., “Highly efficient generation of heritable zebrafish gene mutations using homo- and heterodimeric TALENs”, Nucleic Acids Research, vol. 40, pp. 8001-8010 (2012), and Cermak et al., “Efficient design and assembly of custom TALEN and other TAL effector-based constructs for DNA targeting”, Nucleic Acids Research, vol. 39, No. 17, e82 (2011), each of which are incorporated herein by reference. See also, for example, in Carroll et al., “Genome Engineering with Zinc-Finger Nucleases,” Genetics, August 2011, Vol. 188:773-782; Durai et al., “Zinc finger nucleases: custom-designed molecular scissors for genome engineering of plant and mammalian cells,” Nucleic Acids Res, 2005, Vol. 33:5978-90; and Gaj et al., “ZFN, TALEN, and CRISPR/Cas-based methods for genome engineering,” Trends Biotechnol. 2013, Vol. 31:397-405, each of which are incorporated herein by reference in their entireties.

### Polymerase Domain

In some embodiments, the prime editors disclosed herein comprise a polymerase domain or a variant thereof (e.g., DNA-dependent DNA polymerase or RNA-dependent DNA polymerase, such as, reverse transcriptase). In some cases, the polymerase, or variant thereof, may be provided as a fusion protein with a napDNAbp or other programmable nuclease, or provided in trans.

Any polymerase known in the art may be used in the prime editors with the methods and compositions disclosed herein. The polymerases may be wild type polymerases, functional fragments, mutants, variants, or truncated variants, and the like. The polymerases may include wild type polymerases from eukaryotic, prokaryotic, archael, or viral organisms, and/or the polymerases may be modified by genetic engineering, mutagenesis, or directed evolution-based processes. The polymerases may include T7 DNA polymerase, T5 DNA polymerase, T4 DNA polymerase, Klenow fragment DNA polymerase, DNA polymerase III and the like. The polymerases may also be thermostable, and may include Taq, Tne, Tma, Pfu, Tfl, Tth, Stoffel fragment, VENT® and DEEPVENT® DNA polymerases, KOD, Tgo, JDF3, and mutants, variants and derivatives thereof (see U.S. Pat. Nos. 5,436,149; 4,889,818; 4,965,185; 5,079,352; 5,614,365; 5,374,553; 5,270,179; 5,047,342; 5,512,462; WO 92/06188; WO 92/06200; WO 96/10640; Barnes, W. M., Gene 112:29-35 (1992); Lawyer, F. C., et al., PCR Meth. Appl. 2:275-287 (1993); Flaman, J.-M, et al., Nuc. Acids Res. 22 (15): 3259-3260 (1994), each of which are incorporated by reference).

In some embodiments, the polymerases used in the methods and compositions disclosed herein are “template-dependent” polymerase (since the polymerases are intended to rely on the DNA synthesis template to specify the sequence of the DNA strand under synthesis during prime editing. As used herein, the term “template DNA molecule” refers to that strand of a nucleic acid from which a complementary nucleic acid strand is synthesized by a DNA polymerase, for example, in a primer extension reaction of the DNA synthesis template of a PegRNA.

The disclosure contemplates any wild type polymerase obtained from any naturally-occurring organism or virus, or obtained from a commercial or non-commercial source. In addition, the polymerases usable in the prime editors can include any naturally-occurring mutant polymerase, engineered mutant polymerase, or other variant polymerase, including truncated variants that retain function. The polymerases usable herein may also be engineered to contain specific amino acid substitutions, such as those specifically disclosed herein. In certain preferred embodiments, the polymerases usable in the prime editors utilized in the methods and compositions of the present disclosure are template-based polymerases, i.e., they synthesize nucleotide sequences in a template-dependent manner.

In some embodiments, the polymerase is a DNA polymerase (e.g., a “DNA-dependent DNA polymerase” whereby the template molecule is a strand of DNA). In some embodiments, the polymerase is an RNA polymerase. In various other embodiments, the DNA polymerase can be an “RNA-dependent DNA polymerase” (i.e., whereby the template molecule is a strand of RNA). The term “polymerase” may also refer to an enzyme that catalyzes the polymerization of nucleotide (i.e., the polymerase activity). Generally, the enzyme will initiate synthesis at the 3′-end of a primer annealed to a polynucleotide template sequence (e.g., such as a primer sequence annealed to the primer binding site of a PegRNA), and will proceed toward the 5′ end of the template strand.

In some embodiments, the DNA polymerase is a “functional fragment thereof”. A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

In some embodiments, the polymerase is a reverse transcriptase (RT). RTs are art recognized enzymes with RNA- and DNA-dependent DNA polymerization activity, and an RNaseH activity that catalyzes the cleavage of RNA in RNA-DNA hybrids. In some embodiments, the RT is mutated to disable the RNaseH domain (e.g., to prevent unintended damage to the mRNA). In other embodiments, still, the RNaseH domain is truncated.

Any of the wild type, variant, and/or mutant forms of reverse transcriptases known in the art or which can be made using methods known in the art, such as those described by [[U.S. Pat. No. ______]]], are contemplated herein. For example, in some embodiments, the RT is a wild type RT. Non-limiting examples of RTs include Moloney Murine Leukemia Virus (M-MLV); Human Immunodeficiency Virus (HIV) reverse transcriptase and avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV).

pegRNAs

In some embodiments, the compositions and methods for prime editing contemplated herein comprise at least one pegRNA. Any suitable pegRNA architecture known in the art may be used in any one of the compositions and methods for prime editing disclosed herein, such as those described in U.S. Provisional Application U.S. Ser. No. 63/255,897, U.S. Provisional Application U.S. Ser. No. 63/231,230, U.S. Provisional Application U.S. Ser. No. 63/194,913, U.S. Provisional Application U.S. Ser. No. 63/194,865, U.S. Provisional Application U.S. Ser. No. 63/176,180, U.S. Provisional Application U.S. Ser. No. 63/176,202, and U.S. Provisional Application U.S. Ser. No. 63/136,194, U.S. Provisional Application No. 63/022,397, U.S. Provisional Application No. 63/116,785, International Patent Application No. PCT/US2021/031439, and International Patent Application No. PCT/US2022/012054, the entire contents each of which is incorporated herein by reference in their entireties.

In some embodiments, the pegRNA comprises a spacer sequence, gRNA core, a DNA synthesis template, and a primer binding site. As used herein, the term “spacer sequence” in connection with a guide RNA or a pegRNA refers to the portion of the guide RNA or pegRNA of about 20 nucleotides which contains a nucleotide sequence that shares the same sequence as the protospacer sequence in the target DNA sequence. The spacer sequence anneals to the complement of the protospacer sequence to form a ssRNA/ssDNA hybrid structure at the target site and a corresponding R loop ssDNA structure of the endogenous DNA strand.

In some embodiments, the pegRNA comprises a gRNA core.

In some embodiments, an extended guide RNA usable in the prime editing system utilized in the methods and compositions disclosed herein whereby a traditional guide RNA includes a ~20 nt spacer sequence and a gRNA core region, which binds with the napDNAbp. In this embodiment, the guide RNA includes an extended RNA segment at the 5′ end, i.e., a 5′ extension. In this embodiment, the 5′extension includes a reverse transcription template sequence, a reverse transcription primer binding site, and an optional 5-20 nucleotide linker sequence. The RT primer binding site hybrizes to the free 3′ end that is formed after a nick is formed in the non-target strand of the R-loop, thereby priming reverse transcriptase for DNA polymerization in the 5′-3′ direction.

In another embodiment, an extended guide RNA usable in the prime editing system utilized in the methods and compositions disclosed herein whereby a traditional guide RNA includes a ~20 nt spacer sequence and a gRNA core, which binds with the napDNAbp. In this embodiment, the guide RNA includes an extended RNA segment at the 3′ end, i.e., a 3′ extension. In this embodiment, the 3′extension includes a reverse transcription template sequence, and a reverse transcription primer binding site. The RT primer binding site hybrizes to the free 3′ end that is formed after a nick is formed in the non-target strand of the R-loop, thereby priming reverse transcriptase for DNA polymerization in the 5′-3′ direction.

In another embodiment, an extend guide RNA usable in the prime editing system utilized in the methods and compositions disclosed herein whereby a traditional guide RNA includes a ~20 nt spacer sequence and a gRNA core, which binds with the napDNAbp. In this embodiment, the guide RNA includes an extended RNA segment at an intermolecular position within the gRNA core, i.e., an intramolecular extension. In this embodiment, the intramolecular extension includes a reverse transcription template sequence, and a reverse transcription primer binding site. The RT primer binding site hybrizes to the free 3′ end that is formed after a nick is formed in the non-target strand of the R-loop, thereby priming reverse transcriptase for DNA polymerization in the 5′-3′ direction.

In one embodiment, the position of the intermolecular RNA extension is not in the spacer sequence of the guide RNA. In another embodiment, the position of the intermolecular RNA extension in the gRNA core. In still another embodiment, the position of the intermolecular RNA extension is any position within the guide RNA molecule except within the spacer sequence, or at a position which disrupts the spacer sequence. In one embodiment, the intermolecular RNA extension is inserted downstream from the 3′ end of the spacer sequence. In another embodiment, the intermolecular RNA extension is inserted at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides downstream of the 3′ end of the spacer sequence.

In other embodiments, the intermolecular RNA extension is inserted into the gRNA, which refers to the portion of the guide RNA corresponding or comprising the tracrRNA, which binds and/or interacts with the Cas9 protein or equivalent thereof (i.e, a different napDNAbp). Preferably the insertion of the intermolecular RNA extension does not disrupt or minimally disrupts the interaction between the tracrRNA portion and the napDNAbp.

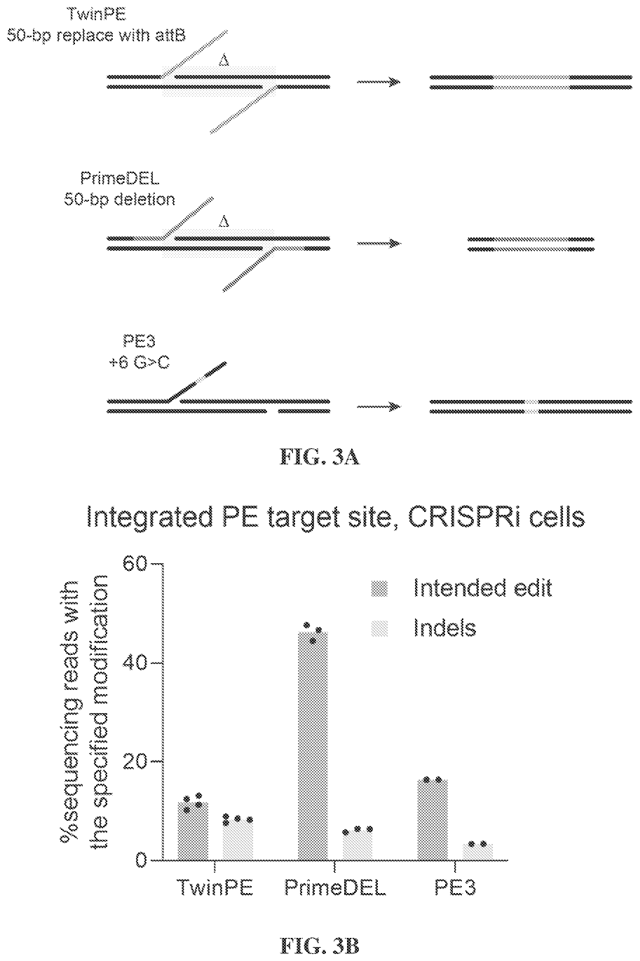

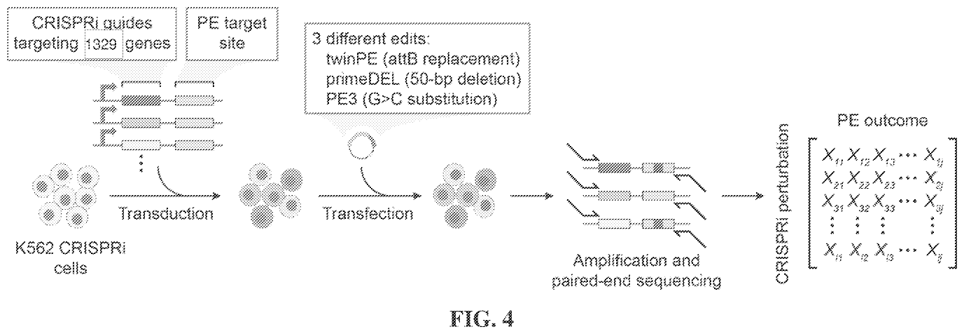

The length of the RNA extension (which includes at least the RT template and primer binding site, e.g., see FIG. 3) can be any useful length. In various embodiments, the RNA extension is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence can also be any suitable length. For example, the RT template sequence can be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In still other embodiments, wherein the reverse transcription primer binding site sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In other embodiments, the optional linker or spacer sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence, in certain embodiments, encodes a single-stranded DNA molecule which is homologous to the non-target strand (and thus, complementary to the corresponding site of the target strand) but includes one or more nucleotide changes. The least one nucleotide change may include one or more single-base nucleotide changes, one or more deletions, and one or more insertions.

The synthesized single-stranded DNA product of the RT template sequence is homologous to the non-target strand and contains one or more nucleotide changes. The single-stranded DNA product of the RT template sequence hybridizes in equilibrium with the complementary target strand sequence, thereby displacing the homologous endogenous target strand sequence. The displaced endogenous strand may be referred to in some embodiments as a 5′ endogenous DNA flap species. This 5′ endogenous DNA flap species can be removed by a 5′ flap endonuclease (e.g., FEN1) and the single-stranded DNA product, now hybridized to the endogenous target strand, may be ligated, thereby creating a mismatch between the endogenous sequence and the newly synthesized strand. The mismatch may be resolved by the cell's innate DNA repair and/or replication processes.

In various embodiments, the nucleotide sequence of the RT template sequence corresponds to the nucleotide sequence of the non-target strand which becomes displaced as the 5′ flap species and which overlaps with the site to be edited.

In various embodiments of the extended guide RNAs, the reverse transcription template sequence may encode a single-strand DNA flap that is complementary to an endogenous DNA sequence adjacent to a nick site, wherein the single-strand DNA flap comprises a desired nucleotide change. The single-stranded DNA flap may displace an endogenous single-strand DNA at the nick site. The displaced endogenous single-strand DNA at the nick site can have a 5′ end and form an endogenous flap, which can be excised by the cell. In various embodiments, excision of the 5′ end endogenous flap can help drive product formation since removing the 5′ end endogenous flap encourages hybridization of the single-strand 3′ DNA flap to the corresponding complementary DNA strand, and the incorporation or assimilation of the desired nucleotide change carried by the single-strand 3′ DNA flap into the target DNA.

In various embodiments of the extended guide RNAs, the cellular repair of the single-strand DNA flap results in installation of the desired nucleotide change, thereby forming a desired product.

In still other embodiments, the desired nucleotide change is installed in an editing window that is between about-5 to +5 of the nick site, or between about-10 to +10 of the nick site, or between about-20 to +20 of the nick site, or between about-30 to +30 of the nick site, or between about-40 to +40 of the nick site, or between about-50 to +50 of the nick site, or between about-60 to +60 of the nick site, or between about-70 to +70 of the nick site, or between about-80 to +80 of the nick site, or between about-90 to +90 of the nick site, or between about-100 to +100 of the nick site, or between about-200 to +200 of the nick site.

In other embodiments, the desired nucleotide change is installed in an editing window that is between about +1 to +2 from the nick site, or about +1 to +3, +1 to +4, +1 to +5, +1 to +6, +1 to +7, +1 to +8, +1 to +9, +1 to +10, +1 to +11, +1 to +12, +1 to +13, +1 to +14, +1 to +15, +1 to +16, +1 to +17, +1 to +18, +1 to +19, +1 to +20, +1 to +21, +1 to +22, +1 to +23, +1 to +24, +1 to +25, +1 to +26, +1 to +27, +1 to +28, +1 to +29, +1 to +30, +1 to +31, +1 to +32, +1 to +33, +1 to +34, +1 to +35, +1 to +36, +1 to +37, +1 to +38, +1 to +39, +1 to +40, +1 to +41, +1 to +42, +1 to +43, +1 to +44, +1 to +45, +1 to +46, +1 to +47, +1 to +48, +1 to +49, +1 to +50, +1 to +51, +1 to +52, +1 to +53, +1 to +54, +1 to +55, +1 to +56, +1 to +57, +1 to +58, +1 to +59, +1 to +60, +1 to +61, +1 to +62, +1 to +63, +1 to +64, +1 to +65, +1 to +66, +1 to +67, +1 to +68, +1 to +69, +1 to +70, +1 to +71, +1 to +72, +1 to +73, +1 to +74, +1 to +75, +1 to +76, +1 to +77, +1 to +78, +1 to +79, +1 to +80, +1 to +81, +1 to +82, +1 to +83, +1 to +84, +1 to +85, +1 to +86, +1 to +87, +1 to +88, +1 to +89, +1 to +90, +1 to +90, +1 to +91, +1 to +92, +1 to +93, +1 to +94, +1 to +95, +1 to +96, +1 to +97, +1 to +98, +1 to +99, +1 to +100, +1 to +101, +1 to +102, +1 to +103, +1 to +104, +1 to +105, +1 to +106, +1 to +107, +1 to +108, +1 to +109, +1 to +110, +1 to +111, +1 to +112, +1 to +113, +1 to +114, +1 to +115, +1 to +116, +1 to +117, +1 to +118, +1 to +119, +1 to +120, +1 to +121, +1 to +122, +1 to +123, +1 to +124, or +1 to +125 from the nick site.

In still other embodiments, the desired nucleotide change is installed in an editing window that is between about +1 to +2 from the nick site, or about +1 to +5, +1 to +10, +1 to +15, +1 to +20, +1 to +25, +1 to +30, +1 to +35, +1 to +40, +1 to +45, +1 to +50, +1 to +55, +1 to +100, +1 to +105, +1 to +110, +1 to +115, +1 to +120, +1 to +125, +1 to +130, +1 to +135, +1 to +140, +1 to +145, +1 to +150, +1 to +155, +1 to +160, +1 to +165, +1 to +170, +1 to +175, +1 to +180, +1 to +185, +1 to +190, +1 to +195, or +1 to +200, from the nick site.

In various aspects, the extended guide RNAs are modified versions of a guide RNA. Guide RNAs maybe naturally occurring, expressed from an encoding nucleic acid, or synthesized chemically. Methods are well known in the art for obtaining or otherwise synthesizing guide RNAs and for determining the appropriate sequence of the guide RNA, including the spacer sequence which interacts and hybridizes with the target strand of a genomic target site of interest.

In various embodiments, the particular design aspects of a guide RNA sequence will depend upon the nucleotide sequence of a genomic target site of interest (i.e., the desired site to be edited) and the type of napDNAbp (e.g., Cas9 protein) present in the prime editing systems utilized in the methods and compositions described herein, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of a napDNAbp (e.g., a Cas9, Cas9 homolog, or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a prime editor to a target sequence may be assessed by any suitable assay. For example, the components of a prime editor, including the guide sequence to be tested, may be provided to a host cell having the corresponding target sequence, such as by transfection with vectors encoding the components of a prime editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a prime editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG (SEQ ID NO: 173) where NNNNNNNNNNNNXGG (SEQ ID NO: 174) (N is A, G, T, or C; and X can be anything). A unique target sequence in a genome may include an S. pyogenes Cas9 target site of the form MMMMMMMMMNNNNNNNNNNNXGG (SEQ ID NO: 175) where NNNNNNNNNNNXGG (SEQ ID NO: 176) (N is A, G, T, or C; and X can be anything). For the S. thermophilus CRISPR1Cas9, a unique target sequence in a genome may include a Cas9 target site of the form MMMMMMMMNNNNNNNNNNNNXXAGAAW (SEQ ID NO: 177) where NNNNNNNNNNNNXXAGAAW (SEQ ID NO: 178) (N is A, G, T, or C; X can be anything; and W is A or T). A unique target sequence in a genome may include an S. thermophilus CRISPR 1 Cas9 target site of the form MMMMMMMMMNNNNNNNNNNNXXAGAAW (SEQ ID NO: 179) where NNNNNNNNNNNXXAGAAW (SEQ ID NO: 180) (N is A, G, T, or C; X can be anything; and W is A or T). For the S. pyogenes Cas9, a unique target sequence in a genome may include a Cas9 target site of the form MMMMMMMMNNNNNNNNNNNNXGGXG (SEQ ID NO: 181) where NNNNNNNNNNNNXGGXG (SEQ ID NO: 182) (N is A, G, T, or C; and X can be anything). A unique target sequence in a genome may include an S. pyogenes Cas9 target site of the form MMMMMMMMMNNNNNNNNNNNXGGXG (SEQ ID NO: 183) where NNNNNNNNNNNXGGXG (SEQ ID NO: 184) (N is A, G, T, or C; and X can be anything). In each of these sequences “M” may be A, G, T, or C, and need not be considered in identifying a sequence as unique.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106 (1): 23-24; and PA Carr and GM Church, 2009, Nature Biotechnology 27 (12): 1151-62). Further algorithms may be found in U.S. application Ser. No. 61/836,080; Broad Reference BI-2013/004A); incorporated herein by reference.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator:

(1)
(SEQ ID NO: 185)
NNNNNNNNGTTTTTGTACTCTCAAGATTTAGAAATAAATCTTGCA
GAAGCTACAAAGATAAGGCTTCATGCCGAAATCAACACCCTGTCA
TTTTATGGCAGGGTGTTTTCGTTATTTAATTTTTT;
(2)
(SEQ ID NO: 186)
NNNNNNNNNNNNNNNNNNGTTTTTGTACTCTCAGAAATGCAGAAG
CTACAAAGATAAGGCTTCATGCCGAAATCAACACCCTGTCATTTT
ATGGCAGGGTGTTTTCGTTATTTAATTTTTT;
(3)
(SEQ ID NO: 187)
NNNNNNNNNNNNNNNNNNNNGTTTTTGTACTCTCAGAAATGCAGA
AGCTACAAAGATAAGGCTTCATGCCGAAATCAACACCCTGTCATT
TTATGGCAGGGTGTTTTTT;
(4)
(SEQ ID NO: 188)
NNNNNNNNNNNNNNNNNNNNGTTTTAGAGCTAGAAATAGCAAGTT
AAAATAAGGCTAGTCCGTTATCAACTTGAAAAAGTGGCACCGAGT
CGGTGCTTTTTT;
(5)
(SEQ ID NO: 189)
NNNNNNNNNNNNNNNNNNNNGTTTTAGAGCTAGAAATAGCAAGTT
AAAATAAGGCTAGTCCGTTATCAACTTGAAAAAGTGTTTTTTT;
AND
(6)
(SEQ ID NO: 190)
NNNNNNNNNNNNNNNNNNNNGTTTTAGAGCTAGAAATAGCAAGTT
AAAATAAGGCTAGTCCGTTATCATTTTTTTT

In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

It will be apparent to those of skill in the art that in order to target any of the fusion proteins comprising a Cas9 domain and a single-stranded DNA binding protein, as disclosed herein, to a target site, e.g., a site comprising a point mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9: nucleic acid editing enzyme/domain fusion protein.

In some embodiments, the guide RNA comprises a structure 5′-[guide sequence]-GUUUUAGAGCUAGAAAUAGCAAGUUAAAAUAAAGGCUAGUCCGUUAUCAACU UGAAAAAGUGGCACCGAGUCGGUGCUUUUU-3′ (SEQ ID NO: 191), wherein the guide sequence comprises a sequence that is complementary to the target sequence. The guide sequence is typically 20 nucleotides long. The sequences of suitable guide RNAs for targeting Cas9: nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein. Additional guide sequences are well known in the art and can be used with the prime editors utilized in the methods and compositions described herein.

In some embodiments, a pegRNA comprises three main component elements ordered in the 5′ to 3′ direction, namely: a spacer, a gRNA core, and an extension arm at the 3′ end. The extension arm may further be divided into the following structural elements in the 5′ to 3′ direction, namely: a primer binding site (A), an edit template (B), and a homology arm (C). In addition, the pegRNA may comprise an optional 3′ end modifier region (e1) and an optional 5′ end modifier region (e2). Still further, the pegRNA may comprise a transcriptional termination signal at the 3′ end of the pegRNA (not depicted). These structural elements are further defined herein. The depiction of the structure of the pegRNA is not meant to be limiting and embraces variations in the arrangement of the elements. For example, the optional sequence modifiers (e1) and (e2) could be positioned within or between any of the other regions shown, and not limited to being located at the 3′ and 5′ ends.

In some embodiments, a pegRNA contemplated herein and may be designed in accordance with the methodology defined in Example 2. The pegRNA comprises three main component elements ordered in the 5′ to 3′ direction, namely: a spacer, a gRNA core, and an extension arm at the 3′ end. The extension arm may further be divided into the following structural elements in the 5′ to 3′ direction, namely: a primer binding site (A), an edit template (B), and a homology arm (C). In addition, the pegRNA may comprise an optional 3′ end modifier region (e1) and an optional 5′ end modifier region (e2). Still further, the pegRNA may comprise a transcriptional termination signal on the 3′ end of the pegRNA (not depicted). These structural elements are further defined herein. The depiction of the structure of the pegRNA is not meant to be limiting and embraces variations in the arrangement of the elements. For example, the optional sequence modifiers (e1) and (e2) could be positioned within or between any of the other regions shown, and not limited to being located at the 3′ and 5′ ends.

The pegRNAs may also include additional design improvements that may modify the properties and/or characteristics of pegRNAs thereby improving the efficacy of prime editing. In various embodiments, these improvements may belong to one or more of a number of different categories, including but not limited to: (1) designs to enable efficient expression of functional pegRNAs from non-polymerase III (pol III) promoters, which would enable the expression of longer pegRNAs without burdensome sequence requirements; (2) improvements to the core, Cas9-binding pegRNA scaffold, which could improve efficacy; (3) modifications to the pegRNA to improve RT processivity, enabling the insertion of longer sequences at targeted genomic loci; and (4) addition of RNA motifs to the 5′ or 3′ termini of the pegRNA that improve pegRNA stability, enhance RT processivity, prevent misfolding of the pegRNA, or recruit additional factors important for genome editing.

In one embodiment, pegRNA could be designed with polIII promoters to improve the expression of longer-length pegRNA with larger extension arms. sgRNAs are typically expressed from the U6 snRNA promoter. This promoter recruits pol III to express the associated RNA and is useful for expression of short RNAs that are retained within the nucleus. However, pol III is not highly processive and is unable to express RNAs longer than a few hundred nucleotides in length at the levels required for efficient genome editing. Additionally, pol III can stall or terminate at stretches of U's, potentially limiting the sequence diversity that could be inserted using a pegRNA. Other promoters that recruit polymerase II (such as pCMV) or polymerase I (such as the U1 snRNA promoter) have been examined for their ability to express longer sgRNAs. However, these promoters are typically partially transcribed, which would result in extra sequence 5′ of the spacer in the expressed pegRNA, which has been shown to result in markedly reduced Cas9: sgRNA activity in a site-dependent manner. Additionally, while pol III-transcribed pegRNAs can simply terminate in a run of 6-7 U's, pegRNAs transcribed from pol II or pol I would require a different termination signal. Often such signals also result in polyadenylation, which would result in undesired transport of the pegRNA from the nucleus. Similarly, RNAs expressed from pol II promoters such as pCMV are typically 5′-capped, also resulting in their nuclear export.

Previously, Rinn and coworkers screened a variety of expression platforms for the production of long-noncoding RNA-(lncRNA) tagged sgRNAs¹⁸³. These platforms include RNAs expressed from pCMV and that terminate in the ENE element from the MALAT1 ncRNA from humans¹⁸⁴, the PAN ENE element from KSHV¹⁸⁵, or the 3′ box from U1 snRNA¹⁸⁶. Notably, the MALAT1 ncRNA and PAN ENEs form triple helices protecting the poly A-tail 184, 187. These constructs could also enhance RNA stability. It is contemplated that these expression systems will also enable the expression of longer pegRNAs.

In addition, a series of methods have been designed for the cleavage of the portion of the pol II promoter that would be transcribed as part of the pegRNA, adding either a self-cleaving ribozyme such as the hammerhead¹⁸⁸, pistol¹⁸⁹, hatchet¹⁸⁹, hairpin¹⁹⁰, VS¹⁹¹, twister¹⁹², or twister sister¹⁹² ribozymes, or other self-cleaving elements to process the transcribed guide, or a hairpin that is recognized by Csy⁴¹⁹³ and also leads to processing of the guide. Also, it is hypothesized that incorporation of multiple ENE motifs could lead to improved pegRNA expression and stability, as previously demonstrated for the KSHV PAN RNA and element¹⁸⁵. It is also anticipated that circularizing the pegRNA in the form of a circular intronic RNA (ciRNA) could also lead to enhanced RNA expression and stability, as well as nuclear localization¹⁹⁴.

In various other embodiments, the pegRNA may be improved by introducing improvements to the scaffold or core sequences. This can be done by introducing known The core, Cas9-binding pegRNA scaffold can likely be improved to enhance PE activity. Several such approaches have already been demonstrated. For instance, the first pairing element of the scaffold (P1) contains a GTTTT-AAAAC pairing element. Such runs of Ts have been shown to result in pol III pausing and premature termination of the RNA transcript. Rational mutation of one of the T-A pairs to a G-C pair in this portion of P1 has been shown to enhance sgRNA activity, suggesting this approach would also be feasible for pegRNAs¹⁹⁵. Additionally, increasing the length of P1 has also been shown to enhance sgRNA folding and lead to improved activity¹⁹⁵, suggesting it as another avenue for the improvement of pegRNA activity

In various other embodiments, the pegRNA may be improved by introducing modifications to the edit template region. As the size of the insertion templated by the pegRNA increases, it is more likely to be degraded by endonucleases, undergo spontaneous hydrolysis, or fold into secondary structures unable to be reverse-transcribed by the RT or that disrupt folding of the pegRNA scaffold and subsequent Cas9-RT binding. Accordingly, it is likely that modification to the template of the pegRNA might be necessary to affect large insertions, such as the insertion of whole genes. Some strategies to do so include the incorporation of modified nucleotides within a synthetic or semi-synthetic pegRNA that render the RNA more resistant to degradation or hydrolysis or less likely to adopt inhibitory secondary structures¹⁹⁶. Such modifications could include 8-aza-7-deazaguanosine, which would reduce RNA secondary structure in G-rich sequences; locked-nucleic acids (LNA) that reduce degradation and enhance certain kinds of RNA secondary structure; 2′-O-methyl, 2′-fluoro, or 2′-O-methoxyethoxy modifications that enhance RNA stability. Such modifications could also be included elsewhere in the pegRNA to enhance stability and activity. Alternatively or additionally, the template of the pegRNA could be designed such that it both encodes for a desired protein product and is also more likely to adopt simple secondary structures that are able to be unfolded by the RT. Such simple structures would act as a thermodynamic sink, making it less likely that more complicated structures that would prevent reverse transcription would occur. Finally, one could also split the template into two, separate pegRNAs. In such a design, a PE would be used to initiate transcription and also recruit a separate template RNA to the targeted site via an RNA-binding protein fused to Cas9 or an RNA recognition element on the pegRNA itself such as the MS2 aptamer. The RT could either directly bind to this separate template RNA, or initiate reverse transcription on the original pegRNA before swapping to the second template. Such an approach could enable long insertions by both preventing misfolding of the pegRNA upon addition of the long template and also by not requiring dissociation of Cas9 from the genome for long insertions to occur, which could possibly be inhibiting PE-based long insertions.

In still other embodiments, the pegRNA may be improved by introducing additional RNA motifs at the 5′ and 3′ termini of the pegRNAs, or even at positions therein between (e.g., in the gRNA core region, or the spacer). Several such motifs-such as the PAN ENE from KSHV and the ENE from MALAT1 were discussed above as possible means to terminate expression of longer pegRNAs from non-pol III promoters. These elements form RNA triple helices that engulf the polyA tail, resulting in their being retained within the nucleus^{184, 187}. However, by forming complex structures at the 3′ terminus of the pegRNA that occlude the terminal nucleotide, these structures would also likely help prevent exonuclease-mediated degradation of pegRNAs.

Other structural elements inserted at the 3′ terminus could also enhance RNA stability, albeit without enabling termination from non-pol III promoters. Such motifs could include hairpins or RNA quadruplexes that would occlude the 3′ terminus¹⁹⁷, or self-cleaving ribozymes such as HDV that would result in the formation of a 2′-3′-cyclic phosphate at the 3′ terminus and also potentially render the pegRNA less likely to be degraded by exonucleases¹⁹⁸. Inducing the pegRNA to cyclize via incomplete splicing—to form a ciRNA—could also increase pegRNA stability and result in the pegRNA being retained within the nucleus 194.

Additional RNA motifs could also improve RT processivity or enhance pegRNA activity by enhancing RT binding to the DNA-RNA duplex. Addition of the native sequence bound by the RT in its cognate retroviral genome could enhance RT activity¹⁹⁹. This could include the native primer binding site (PBS), polypurine tract (PPT), or kissing loops involved in retroviral genome dimerization and initiation of transcription¹⁹⁹.

Addition of dimerization motifs-such as kissing loops or a GNRA tetraloop/tetraloop receptor pair²⁰⁰—at the 5′ and 3′ termini of the pegRNA could also result in effective circularization of the pegRNA, improving stability. Additionally, it is envisioned that addition of these motifs could enable the physical separation of the pegRNA spacer and primer, prevention occlusion of the spacer which would hinder PE activity. Short 5′ extensions or 3′ extensions to the pegRNA that form a small toehold hairpin in the spacer region or along the primer binding site could also compete favorably against the annealing of intracomplementary regions along the length of the pegRNA, e.g., the interaction between the spacer and the primer binding site that can occur. Finally, kissing loops could also be used to recruit other template RNAs to the genomic site and enable swapping of RT activity from one RNA to the other.

pegRNA scaffolds could be further improved via directed evolution, in an analogous fashion to how SpCas9 and prime editors (PE) have been improved. Directed evolution could enhance pegRNA recognition by Cas9 or evolved Cas9 variants. Additionally, it is likely that different pegRNA scaffold sequences would be optimal at different genomic loci, either enhancing PE activity at the site in question, reducing off-target activities, or both. Finally, evolution of pegRNA scaffolds to which other RNA motifs have been added would almost certainly improve the activity of the fused pegRNA relative to the unevolved, fusion RNA. For instance, evolution of allosteric ribozymes composed of c-di-GMP-I aptamers and hammerhead ribozymes led to dramatically improved activity²⁰², suggesting that evolution would improve the activity of hammerhead-pegRNA fusions as well. In addition, while Cas9 currently does not generally tolerate 5′ extension of the sgRNA, directed evolution will likely generate enabling mutations that mitigate this intolerance, allowing additional RNA motifs to be utilized.

The present disclosure contemplates any such ways to further improve the efficacy of the prime editing systems utilized in the methods and compositions disclosed here.

In various embodiments, it may be advantageous to limit the appearance of consecutive sequence of Ts from the extension arm as consecutive series of T's may limit the capacity of the pegRNA to be transcribed. For example, strings of at least consecutive three T's, at least consecutive four T's, at least consecutive five T's, at least consecutive six T's, at least consecutive seven T's, at least consecutive eight T's, at least consecutive nine T's, at least consecutive ten T's, at least consecutive eleven T's, at least consecutive twelve T's, at least consecutive thirteen T's, at least consecutive fourteen T's, or at least consecutive fifteen T's should be avoided when designing the pegRNA, or should be at least removed from the final designed sequence. In one embodiment, one can avoid the includes of unwanted strings of consecutive T's in pegRNA extension arms but avoiding target sites that are rich in consecutive A: T nucleobase pairs.

### Linkers, NLS, and Other PE Elements

### Linkers

As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and the catalytic domain of a polymerase (e.g., a reverse transcriptase). In some embodiments, a linker joins a dCas9 and reverse transcriptase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included funtionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)ₙ (SEQ ID NO: 118), (G)ₙ (SEQ ID NO: 119), (EAAAK)ₙ (SEQ ID NO: 120), (GGS)ₙ (SEQ ID NO: 121), (SGGS)ₙ (SEQ ID NO: 122), (XP)ₙ (SEQ ID NO: 123), or any combination thereof, wherein n is independently an integer between 1 and 30, and wherein X is any amino acid. In some embodiments, the linker comprises the amino acid sequence (GGS)ₙ (SEQ ID NO: 121), wherein n is 1, 3, or 7. In some embodiments, the linker comprises the amino acid sequence SGSETPGTSESATPES (SEQ ID NO: 124). In some embodiments, the linker comprises the amino acid sequence SGGSSGGSSGSETPGTSESATPESSGGSSGGS (SEQ ID NO: 125). In some embodiments, the linker comprises the amino acid sequence SGGSGGSGGS (SEQ ID NO: 126). In some embodiments, the linker comprises the amino acid sequence SGGS (SEQ ID NO: 127). In other embodiments, the linker comprises the amino acid sequence

(SEQ ID NO: 128, 60AA)
SGGSSGGSSGSETPGTSESATPESAGSYPYDVPDYAGSAAPAAKK
KKLDGSGSGGSSGGS.

In certain embodiments, linkers may be used to link any of the peptides or peptide domains or moieties of the invention (e.g., a napDNAbp linked or fused to a reverse transcriptase).

As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and the catalytic domain of a recombinase. In some embodiments, a linker joins a dCas9 and reverse transcriptase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminoHEXAnoic acid (Ahx). In certain embodiments, the linker is based on a carbocyclic moiety (e.g., cyclopentane, cycloHEXAne). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)n (SEQ ID NO: 118), (G)n (SEQ ID NO: 119), (EAAAK)ₙ (SEQ ID NO: 120), (GGS)ₙ (SEQ ID NO: 121), (SGGS)n (SEQ ID NO: 122), (XP)n (SEQ ID NO: 123), or any combination thereof, wherein n is independently an integer between 1 and 30, and wherein X is any amino acid. In some embodiments, the linker comprises the amino acid sequence (GGS)n (SEQ ID NO: 121), wherein n is 1, 3, or 7. In some embodiments, the linker comprises the amino acid sequence SGSETPGTSESATPES (SEQ ID NO: 124). In some embodiments, the linker comprises the amino acid sequence SGGSSGGSSGSETPGTSESATPESSGGSSGGS (SEQ ID NO: 125). In some embodiments, the linker comprises the amino acid sequence SGGSGGSGGS (SEQ ID NO: 126). In some embodiments, the linker comprises the amino acid sequence SGGS (SEQ ID NO: 127).

In particular, the following linkers can be used in various embodiments to join prime editor domains with one another:

(SEQ ID NO: 129)
GGS;
(SEQ ID NO: 130)
GGSGGS;
(SEQ ID NO: 131)
GGSGGSGGS;
(SEQ ID NO: 102)
SGGSSGGSSGSETPGTSESATPESSGGSSGGSS;
(SEQ ID NO: 124)
SGSETPGTSESATPES;
(SEQ ID NO: 128)
SGGSSGGSSGSETPGTSESATPESAGSYPYDVPDYAGS
AAPAAKKKKLDGSGSGGSSGG S

The PE fusion proteins may also comprise various other domains besides the napDNAbp (e.g., Cas9 domain) and the polymerase domain (e.g., RT domain). For example, in the case where the napDNAbp is a Cas9 and the polymerase is a RT, the PE fusion proteins may comprise one or more linkers that join the Cas9 domain with the RT domain. The linkers may also join other functional domains, such as nuclear localization sequences (NLS) or a FEN1 (or other flap endonuclease) to the PE fusion proteins or a domain thereof.

### Nuclear Localization Sequences (NLS)

In various embodiments, the PE fusion proteins may comprise one or more nuclear localization sequences (NLS), which help promote translocation of a protein into the cell nucleus. Such sequences are well-known in the art and can include the following examples:

| DESCRIPTION | SEQUENCE | SEQ ID NO: |
|---|---|---|
| NLS OF | PKKKRKV | SEQ ID NO: 132 |
| SV40 | | |
| LARGE T- | | |
| AG | | |
| NLS | MKRTADGSEFESPKKKRKV | SEQ ID NO: 101 |
| NLS | MDSLLMNRRKFLYQFKNVR | SEQ ID NO: 1 |
| | WAKGRRETYLC | |
| NLS OF | AVKRPAATKKAGQAKKKKL | SEQ ID NO: 133 |
| NUCLEOP | D | |
| LASMIN | | |
| NLS OF | MSRRRKANPTKLSENAKKLA | SEQ ID NO: 134 |
| EGL-13 | KEVEN | |
| NLS OF C- | PAAKRVKLD | SEQ ID NO: 135 |
| MYC | | |
| NLS OF | KLKIKRPVK | SEQ ID NO: 136 |
| TUS- | | |
| PROTEIN | | |
| NLS OF | VSRKRPRP | SEQ ID NO: 137 |
| POLYOMA | | |
| LARGE T- | | |
| AG | | |
| NLS OF | EGAPPAKRAR | SEQ ID NO: 138 |
| HEPATITIS | | |
| D VIRUS | | |
| ANTIGEN | | |
| NLS OF | PPQPKKKPLDGE | SEQ ID NO: 139 |
| MURINE | | |
| P53 | | |
| NLS OF | SGGSKRTADGSEFEPKKKRKV | SEQ ID NO: 103 |
| PE1 AND | | |
| PE2 | | |

The NLS examples above are non-limiting. The PE fusion proteins may comprise any known NLS sequence, including any of those described in Cokol et al., “Finding nuclear localization signals,” EMBO Rep., 2000, 1 (5): 411-415 and Freitas et al., “Mechanisms and Signals for the Nuclear Import of Proteins,” Current Genomics, 2009, 10 (8): 550-7, each of which are incorporated herein by reference.

In various embodiments, the prime editors and constructs encoding the prime editors utilized in the methods and compositions disclosed herein further comprise one or more, preferably, at least two nuclear localization signals. In certain embodiments, the prime editors comprise at least two NLSs. In embodiments with at least two NLSs, the NLSs can be the same NLSs or they can be different NLSs. In addition, the NLSs may be expressed as part of a fusion protein with the remaining portions of the prime editors. In some embodiments, one or more of the NLSs are bipartite NLSs (“bpNLS”). In certain embodiments, the disclosed fusion proteins comprise two bipartite NLSs. In some embodiments, the disclosed fusion proteins comprise more than two bipartite NLSs.

The location of the NLS fusion can be at the N-terminus, the C-terminus, or within a sequence of a prime editor (e.g., inserted between the encoded napDNAbp component (e.g., Cas9) and a polymerase domain (e.g., a reverse transcriptase domain).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 132), MDSLLMNRRKFLYQFKNVRWAKGRRETYLC (SEQ ID NO: 1), KRTADGSEFESPKKKRKV (SEQ ID NO: 140), or KRTADGSEFEPKKKRKV (SEQ ID NO: 141). In other embodiments, NLS comprises the amino acid sequences NLSKRPAAIKKAGQAKKKK (SEQ ID NO: 142), PAAKRVKLD (SEQ ID NO: 135), RQRRNELKRSF (SEQ ID NO: 143), NQSSNFGPMKGGNFGGRSSGPYGGGGQYFAKPRNQGGY (SEQ ID NO: 144).

In one aspect of the disclosure, a prime editor may be modified with one or more nuclear localization signals (NLS), preferably at least two NLSs. In certain embodiments, the prime editors are modified with two or more NLSs. The disclosure contemplates the use of any nuclear localization signal known in the art at the time of the disclosure, or any nuclear localization signal that is identified or otherwise made available in the state of the art after the time of the instant filing. A representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273:14731-37, incorporated herein by reference) to eight amino acids, and is typically rich in lysine and arginine residues (Magin et al., (2000) Virology 274:11-16, incorporated herein by reference). Nuclear localization signals often comprise proline residues. A variety of nuclear localization signals have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 132)); (ii) a bipartite motif consisting of two basic domains separated by a variable number of spacer amino acids and exemplified by the Xenopus nucleoplasmin NLS (KRXXXXXXXXXXKKKL (SEQ ID NO: 145)); and (iii) noncanonical sequences such as M9 of the hnRNP A1 protein, the influenza virus nucleoprotein NLS, and the yeast Gal4 protein NLS (Dingwall and Laskey 1991).

Nuclear localization signals appear at various points in the amino acid sequences of proteins. NLS's have been identified at the N-terminus, the C-terminus and in the central region of proteins. Thus, the disclosure provides prime editors that may be modified with one or more NLSs at the C-terminus, the N-terminus, as well as at in internal region of the prime editor. The residues of a longer sequence that do not function as component NLS residues should be selected so as not to interfere, for example tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a prime editor to include one or more NLSs. In one aspect, the prime editors may be engineered to express a prime editor protein that is translationally fused at its N-terminus or its C-terminus (or both) to one or more NLSs, i.e., to form a prime editor-NLS fusion construct. In other embodiments, the prime editor-encoding nucleotide sequence may be genetically modified to incorporate a reading frame that encodes one or more NLSs in an internal region of the encoded prime editor. In addition, the NLSs may include various amino acid linkers or spacer regions encoded between the prime editor and the N-terminally, C-terminally, or internally-attached NLS amino acid sequence, e.g, and in the central region of proteins. Thus, the present disclosure also provides for nucleotide constructs, vectors, and host cells for expressing fusion proteins that comprise a prime editor and one or more NLSs.

The prime editors utilized in the methods and compositions described herein may also comprise nuclear localization signals which are linked to a prime editor through one or more linkers, e.g., and polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and be joined to the prime editor by any suitable strategy that effectuates forming a bond (e.g., covalent linkage, hydrogen bonding) between the prime editor and the one or more NLSs.

### Flap Endonucleases

In various embodiments, the PE fusion proteins may comprise one or more flap endonucleases (e.g., FEN1), which refers to an enzyme that catalyzes the removal of 5′ single strand DNA flaps. These are naturally occurring enzymes that process the removal of 5′ flaps formed during cellular processes, including DNA replication. The prime editing utilized in the methods and compositions described herein may utilize endogenously supplied flap endonucleases or those provided in trans to remove the 5′ flap of endogenous DNA formed at the target site during prime editing. Flap endonucleases are known in the art and can be found described in Patel et al., “Flap endonucleases pass 5′-flaps through a flexible arch using a disorder-thread-order mechanism to confer specificity for free 5′-ends,” Nucleic Acids Research, 2012, 40 (10): 4507-4519 and Tsutakawa et al., “Human flap endonuclease structures, DNA double-base flipping, and a unified understanding of the FEN1 superfamily,” Cell, 2011, 145 (2): 198-211 (each of which are incorporated herein by reference).

In various embodiments, the prime editor fusion proteins utilized in the methods and compositions contemplated herein may include any flap endonuclease variant of the above-disclosed sequences having an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the above sequences.

Other endonucleases that may be utilized by the instant methods to facilitate removal of the 5′ end single strand DNA flap include, but are not limited to (1) trex 2, (2) exo1 endonuclease (e.g., Keijzers et al., Biosci Rep. 2015, 35 (3): e00206)

### Additional PE Elements

In certain embodiments, the prime editors utilized in the methods and compositions described herein may comprise an inhibitor of base repair. The term “inhibitor of base repair” or “IBR” refers to a protein that is capable in inhibiting the activity of a nucleic acid repair enzyme, for example a base excision repair enzyme. In some embodiments, the IBR is an inhibitor of OGG base excision repair. In some embodiments, the IBR is an inhibitor of base excision repair (“iBER”). Exemplary inhibitors of base excision repair include inhibitors of APE1, Endo III, Endo IV, Endo V, Endo VIII, Fpg, hOGG1, hNEIL1, T7 EndoI, T4PDG, UDG, hSMUG1, and hAAG. In some embodiments, the IBR is an inhibitor of Endo V or hAAG. In some embodiments, the IBR is an iBER that may be a catalytically inactive glycosylase or catalytically inactive dioxygenase or a small molecule or peptide inhibitor of an oxidase, or variants thereof. In some embodiments, the IBR is an iBER that may be a TDG inhibitor, MBD4 inhibitor or an inhibitor of an AlkBH enzyme. In some embodiments, the IBR is an iBER that comprises a catalytically inactive TDG or catalytically inactive MBD4. An exemplary catalytically inactive TDG is an N140A mutant of SEQ ID NO: 172 (human TDG).

In some embodiments, the fusion proteins described herein may comprise one or more heterologous protein domains (e.g., about or more than about 1, 2, 3, 4, 5, 6, 7, 8, 9, 10, or more domains in addition to the prime editor components). A fusion protein may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags that are useful for solubilization, purification, or detection of the fusion proteins.

Examples of protein domains that may be fused to a prime editor or component thereof (e.g., the napDNAbp domain, the polymerase domain, or the NLS domain) include, without limitation, epitope tags, and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A prime editor may be fused to a gene sequence encoding a protein or a fragment of a protein that bind DNA molecules or bind other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a prime editor are described in US Patent Publication No. 2011/0059502, published Mar. 10, 2011 and incorporated herein by reference in its entirety.

In an aspect of the disclosure, a reporter gene which includes, but is not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In certain embodiments of the disclosure the gene product is luciferase. In a further embodiment of the disclosure the expression of the gene product is decreased.

Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FLASH tags, V5 tags, and SBP-tags. Additional suitable sequences will be apparent to those of skill in the art. In some embodiments, the fusion protein comprises one or more His tags.

In some embodiments of the present disclosure, the activity of the prime editing system may be temporally regulated by adjusting the residence time, the amount, and/or the activity of the expressed components of the PE system. For example, as described herein, the PE may be fused with a protein domain that is capable of modifying the intracellular half-life of the PE. In certain embodiments involving two or more vectors (e.g., a vector system in which the components described herein are encoded on two or more separate vectors), the activity of the PE system may be temporally regulated by controlling the timing in which the vectors are delivered. For example, in some embodiments a vector encoding the nuclease system may deliver the PE prior to the vector encoding the template. In other embodiments, the vector encoding the pegRNA may deliver the guide prior to the vector encoding the PE system. In some embodiments, the vectors encoding the PE system and pegRNA are delivered simultaneously. In certain embodiments, the simultaneously delivered vectors temporally deliver, e.g., the PE, pegRNA, and/or second strand guide RNA components. In further embodiments, the RNA (such as, e.g., the nuclease transcript) transcribed from the coding sequence on the vectors may further comprise at least one element that is capable of modifying the intracellular half-life of the RNA and/or modulating translational control. In some embodiments, the half-life of the RNA may be increased. In some embodiments, the half-life of the RNA may be decreased. In some embodiments, the element may be capable of increasing the stability of the RNA. In some embodiments, the element may be capable of decreasing the stability of the RNA. In some embodiments, the element may be within the 3′ UTR of the RNA. In some embodiments, the element may include a polyadenylation signal (PA). In some embodiments, the element may include a cap, e.g., an upstream mRNA or pegRNA end. In some embodiments, the RNA may comprise no PA such that it is subject to quicker degradation in the cell after transcription. In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus (WHP).

Posttranscriptional Regulatory Element (WPRE), which creates a tertiary structure to enhance expression from the transcript. In further embodiments, the element is a modified and/or truncated WPRE sequence that is capable of enhancing expression from the transcript, as described, for example in Zufferey et al., J Virol, 73 (4): 2886-92 (1999) and Flajolet et al., J Virol, 72 (7): 6175-80 (1998). In some embodiments, the WPRE or equivalent may be added to the 3′ UTR of the RNA. In some embodiments, the element may be selected from other RNA sequence motifs that are enriched in either fast- or slow-decaying transcripts.

In some embodiments, the vector encoding the PE or the pegRNA may be self-destroyed via cleavage of a target sequence present on the vector by the PE system. The cleavage may prevent continued transcription of a PE or a pegRNA from the vector. Although transcription may occur on the linearized vector for some amount of time, the expressed transcripts or proteins subject to intracellular degradation will have less time to produce off-target effects without continued supply from expression of the encoding vectors.

### Kits, Cells, and Vectors

### Kits

The compositions of the present disclosure may be assembled into kits. In some embodiments, the kit comprises nucleic acid vectors for the expression of a prime editor and an inhibitor, such as, but not limited to an MLH1 dominant negative variant as described herein. In other embodiments, the kit further comprises appropriate guide nucleotide sequences (e.g., pegRNAs and second-site gRNAs) or nucleic acid vectors for the expression of such guide nucleotide sequences, to target the Cas9 protein or prime editor to the desired target sequence.

The kit described herein may include one or more containers housing components for performing the methods described herein and optionally instructions for use. Any of the kit described herein may further comprise components needed for performing the assay methods. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe and shipped refrigerated. Alternatively it may be housed in a vial or other container for storage. A second container may have other components prepared sterilely. Alternatively the kits may include the active agents premixed and shipped in a vial, tube, or other container.

The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. Some aspects of this disclosure provide kits comprising a nucleic acid construct comprising a nucleotide sequence encoding the various components of the prime editing system utilized in the methods and compositions described herein (e.g., including, but not limited to, the napDNAbps, reverse transcriptases, polymerases, fusion proteins (e.g., comprising napDNAbps and reverse transcriptases (or more broadly, polymerases), extended guide RNAs, and complexes comprising fusion proteins and extended guide RNAs, as well as accessory elements, such as second strand nicking components (e.g., second strand nicking gRNA) and 5′ endogenous DNA flap removal endonucleases for helping to drive the prime editing process towards the edited product formation). In some embodiments, the nucleotide sequence(s) comprises a heterologous promoter (or more than a single promoter) that drives expression of the prime editing system components.

Other aspects of this disclosure provide kits comprising one or more nucleic acid constructs encoding the various components of the prime editing systems utilized in the methods and compositions described herein, e.g., the comprising a nucleotide sequence encoding the components of the prime editing system capable of modifying a target DNA sequence. In some embodiments, the nucleotide sequence comprises a heterologous promoter that drives expression of the prime editing system components.

Some aspects of this disclosure provides kits comprising a nucleic acid construct, comprising (a) a nucleotide sequence encoding a napDNAbp (e.g., a Cas9 domain) fused to a reverse transcriptase and (b) a heterologous promoter that drives expression of the sequence of (a).

### Cells

Cells that may contain any of the compositions described herein include prokaryotic cells and eukaryotic cells. The methods described herein are used to deliver a Cas9 protein or a prime editor and an inhibitor (e.g., an MLH1 dominant negative variant) into a eukaryotic cell (e.g., a mammalian cell, such as a human cell). In some embodiments, the cell is in vitro (e.g., cultured cell. In some embodiments, the cell is in vivo (e.g., in a subject such as a human subject). In some embodiments, the cell is ex vivo (e.g., isolated from a subject and may be administered back to the same or a different subject). Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, rAAV vectors are delivered into human embryonic kidney (HEK) cells (e.g., HEK 293 or HEK 293T cells). In some embodiments, rAAV vectors are delivered into stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepalclc7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Some aspects of this disclosure provide cells comprising any of the constructs disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO—IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

### Vectors

Some aspects of the present disclosure relate to using recombinant virus vectors (e.g., adeno-associated virus vectors, adenovirus vectors, or herpes simplex virus vectors) for the delivery of the prime editors and MLH1 dominant negative mutants as described herein into a cell. In the case of a split-PE approach, the N-terminal portion of a PE fusion protein and the C-terminal portion of a PE fusion are delivered by separate recombinant virus vectors (e.g., adeno-associated virus vectors, adenovirus vectors, or herpes simplex virus vectors) into the same cell, since the full-length Cas9 protein or prime editors exceeds the packaging limit of various virus vectors, e.g., rAAV (~4.9 kb).

In some embodiments, the vectors used herein may encode the PE fusion proteins, or any of the components thereof (e.g., napDNAbp, linkers, or polymerases), or an MLH1 dominant negative mutant. In addition, the vectors used herein may encode the pegRNAs, and/or the accessory gRNA for second strand nicking. The vectors may be capable of driving expression of one or more coding sequences in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild-type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the promoters that may be used in the prime editor vectors may be constitutive, inducible, or tissue-specific. In some embodiments, the promoters may be a constitutive promoters. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EFla) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EFla promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, the promoter may be a tissue-specific promoter. In some embodiments, the tissue-specific promoter is exclusively or predominantly expressed in liver tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-promoter, Mb promoter, Nphsl promoter, OG-2 promoter, SP-B promoter, SYN1 promoter, and WASP promoter.

In some embodiments, the prime editor and MLH1 dominant negative mutant vectors (e.g., including any vectors encoding the prime editor fusion protein and/or the pegRNAs, and/or the accessory second strand nicking gRNAs) may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the prime editor vectors (e.g., including any vectors encoding the prime editor fusion protein and/or the pegRNAs, and/or the accessory second strand nicking gRNAs) and MLH1 dominant negative mutant vector (e.g., any vector encoding an MLH1 dominant negative mutant as described herein) may comprise tissue-specific promoters to start expression only after it is delivered into a specific tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter.

In some embodiments, the nucleotide sequence encoding the pegRNA (or any guide RNAs used in connection with prime editing) may be operably linked to at least one transcriptional or translational control sequence. In some embodiments, the nucleotide sequence encoding the guide RNA may be operably linked to at least one promoter. In some embodiments, the promoter may be recognized by RNA polymerase III (Pol III). Non-limiting examples of Pol III promoters include U6, HI and tRNA promoters. In some embodiments, the nucleotide sequence encoding the guide RNA may be operably linked to a mouse or human U6 promoter. In other embodiments, the nucleotide sequence encoding the guide RNA may be operably linked to a mouse or human HI promoter. In some embodiments, the nucleotide sequence encoding the guide RNA may be operably linked to a mouse or human tRNA promoter. In embodiments with more than one guide RNA, the promoters used to drive expression may be the same or different. In some embodiments, the nucleotide encoding the crRNA of the guide RNA and the nucleotide encoding the tracr RNA of the guide RNA may be provided on the same vector. In some embodiments, the nucleotide encoding the crRNA and the nucleotide encoding the tracr RNA may be driven by the same promoter. In some embodiments, the crRNA and tracr RNA may be transcribed into a single transcript. For example, the crRNA and tracr RNA may be processed from the single transcript to form a double-molecule guide RNA. Alternatively, the crRNA and tracr RNA may be transcribed into a single-molecule guide RNA.

In some embodiments, the nucleotide sequence encoding the guide RNA may be located on the same vector comprising the nucleotide sequence encoding the PE fusion protein. In some embodiments, expression of the guide RNA and of the PE fusion protein may be driven by their corresponding promoters. In some embodiments, expression of the guide RNA may be driven by the same promoter that drives expression of the PE fusion protein. In some embodiments, the guide RNA and the PE fusion protein transcript may be contained within a single transcript. For example, the guide RNA may be within an untranslated region (UTR) of the Cas9 protein transcript. In some embodiments, the guide RNA may be within the 5′ UTR of the PE fusion protein transcript. In other embodiments, the guide RNA may be within the 3′ UTR of the PE fusion protein transcript. In some embodiments, the intracellular half-life of the PE fusion protein transcript may be reduced by containing the guide RNA within its 3′ UTR and thereby shortening the length of its 3′ UTR. In additional embodiments, the guide RNA may be within an intron of the PE fusion protein transcript. In some embodiments, suitable splice sites may be added at the intron within which the guide RNA is located such that the guide RNA is properly spliced out of the transcript. In some embodiments, expression of the Cas9 protein and the guide RNA in close proximity on the same vector may facilitate more efficient formation of the CRISPR complex.

The vector system may comprise one vector, or two vectors, or three vectors, or four vectors, or five vector, or more. In some embodiments, the vector system may comprise one single vector, which encodes both the PE fusion protein, the pegRNA, and an MLH1 dominant negative mutant. In other embodiments, the vector system may comprise two vectors, wherein one vector encodes the PE fusion protein and the pegRNA, and the other encodes the MLH1 dominant negative mutant.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

### Pharmaceutical Compositions

Other aspects of the present disclosure relate to pharmaceutical compositions comprising any of the various prime editing system described herein (e.g., including, but not limited to, the napDNAbps, reverse transcriptases, fusion proteins (e.g., comprising napDNAbps and reverse transcriptases), extended guide RNAs, and complexes comprising fusion proteins and extended guide RNAs, as well as accessory elements, such as second strand nicking components and 5′ endogenous DNA flap removal endonucleases for helping to drive the multi-flap prime editing process towards the edited product formation).

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi) propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### EXAMPLES

### Example 1

### Lentivirus Production

To package lentivirus, HEK293T cells were seeded on 6-well plates (Corning) at 7.5×10⁵ cells per well in DMEM supplemented with 10% FBS. At 60% confluency 16 h after seeding, cells were transfected with 12 μL Lipofectamine 2000 (Thermo Fisher Scientific) according to the manufacturer's protocol and 1.33 μg lentiviral transfer plasmid, 0.67 μg pMD2.G (Addgene #12259), and 1 μg psPAX2 (Addgene #12260). 6 h after transfection, media was exchanged with DMEM supplemented with 10% FBS. 48 h after transfection, viral supernatant was centrifuged at 3000 g for 15 min to remove cellular debris, filtered through a 0.45 μm PVDF filter (Corning), and stored at −80° C.

For larger scale lentivirus production, HEK293T cells were seeded on 6-well plates (Corning) at 7.5×10⁵ cells per well in DMEM supplemented with 10% FBS. At 60% confluency 16 h after seeding, cells were transfected with 12 μL Lipofectamine 2000 (Thermo Fisher Scientific) according to the manufacturer's protocol and 1.33 μg lentiviral transfer plasmid, 0.67 μg pMD2.G (Addgene #12259), and 1 μg psPAX2 (Addgene #12260). 6 h after transfection, media was exchanged with DMEM supplemented with 10% FBS. 48 h after transfection, viral supernatant was centrifuged at 3000 g for 15 min to remove cellular debris, filtered through a 0.45 μm PVDF filter (Corning), and stored at −80° C.

Design and Construction of Lentiviral Screening Vector with CRISPRi sgRNA and Prime Edit Target Site

The lentiviral transfer plasmid backbone for prime editing screens (pPC1655) was designed from the pPC1000 vector backbone¹ to contain a specific prime edit site and express a control GFP-targeting S. pyogenes sgRNA for CRISPRi. The prime edit site comprised a protospacer from the HBB 3′UTR and a protospacer from CCR5. These protospacers were positioned in a PAM-in orientation and were adjacent to a PAM sequence for SaCas9. This 234-bp edit site containing two targets for SaCas9-pegRNAs was then edited by PE3 with a +50 nick, twin prime editing, and PRIME-Del.

This 234-bp edit site was positioned adjacent to an S. pyogenes sgRNA expression cassette driven by a modified mouse U6 promoter such that an sgRNA and edit site could be amplified by PCR in the same 453-bp amplicon. The sgRNA expression cassette in pPC1655 encoded an EGFP-targeting control sgRNA (spacer, 5′-GACCAGGATGGGCACCACCC-3′ (SEQ ID NO: XX)) and an pEF1α-PuroR-T2A-BFP selection marker.

### CRISPRi Library Cloning and Lentiviral Library Production

An oligonucleotide library of CRISPRi sgRNAs was designed to contain 210 non-targeting control sgRNAs and 4,304 sgRNAs that target 1,329 genes involved in DNA repair, DNA replication, DNA metabolism, chromatin binding, chromatin remodeling, nuclease function, and helicase function (See FIG. 4). 1,496 of these gene-targeting sgRNAs were derived from a DNA repair CRISPRi library previously used for Repair-seq². The oligonucleotide library was ordered from Twist Bioscience and sequences were amplified by PCR using Phusion High-Fidelity DNA Polymerase (New England BioLabs) and purified with the NucleoSpin Gel and PCR Clean-up kit (Macherey-Nagel). The amplified sequences and the pPC1000 lentiviral screen vector containing the pre-validated prime edit site were digested with BstXI and BlpI restriction endonucleases (Thermo Fisher Scientific), ligated with T4 ligase (New England BioLabs), and transformed into 10-beta electrocompetent cells (New England BioLabs). The plasmid library was isolated from transformed cells using QIAGEN Plasmid Plus Midi Kit, and the pooled library of plasmids was verified by PCR and sequencing on a MiSeq Reagent Kit v2 (Illumina).

### Prime Editing CRISPRi Screens in K562 Cells

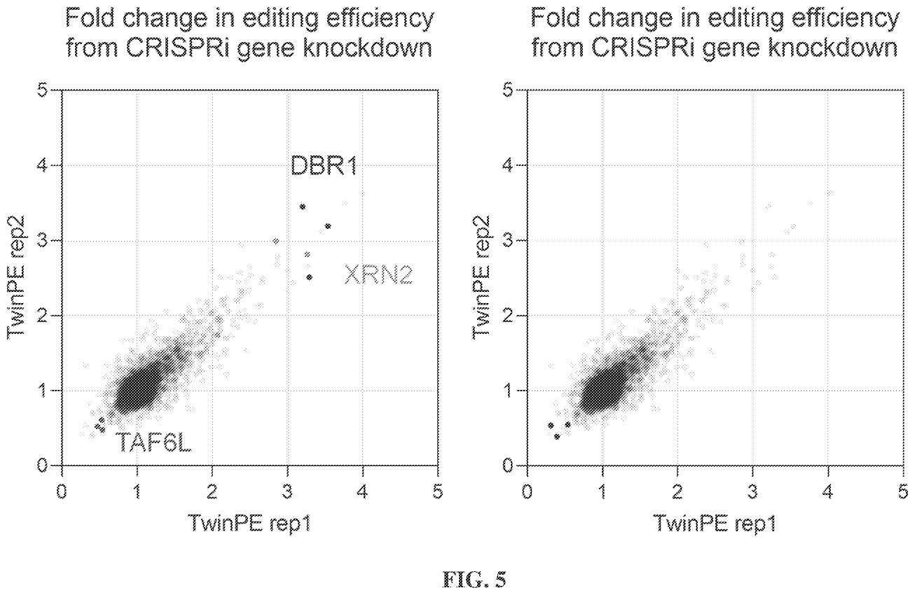

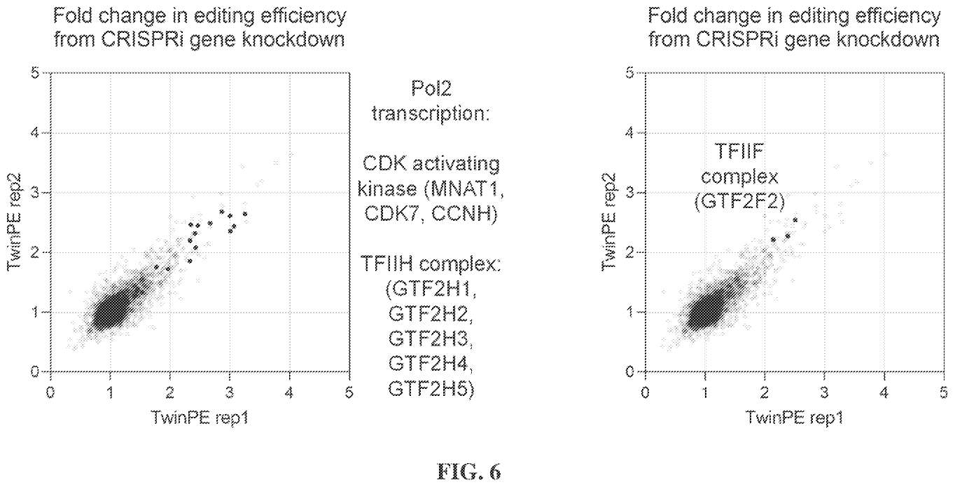

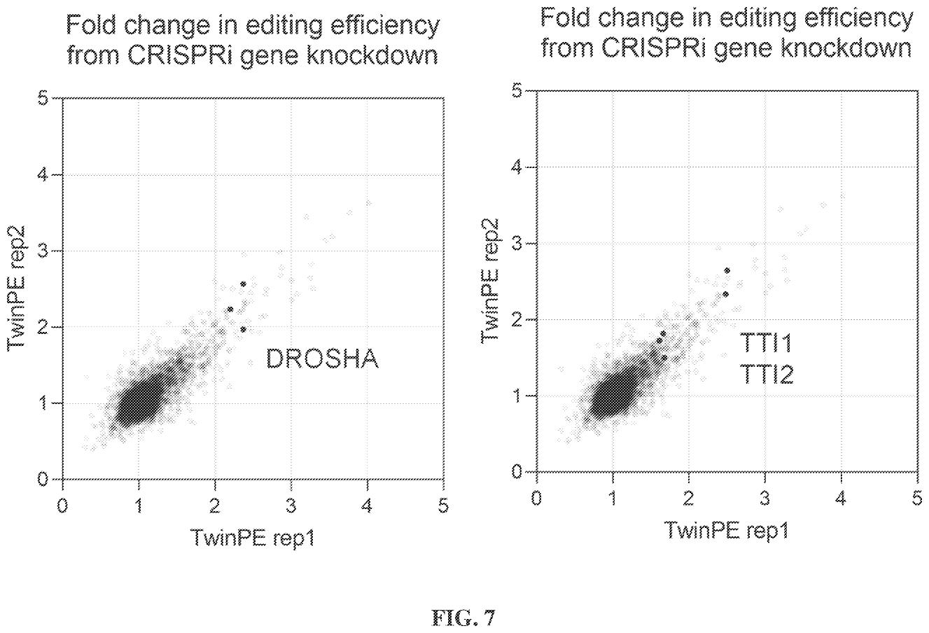

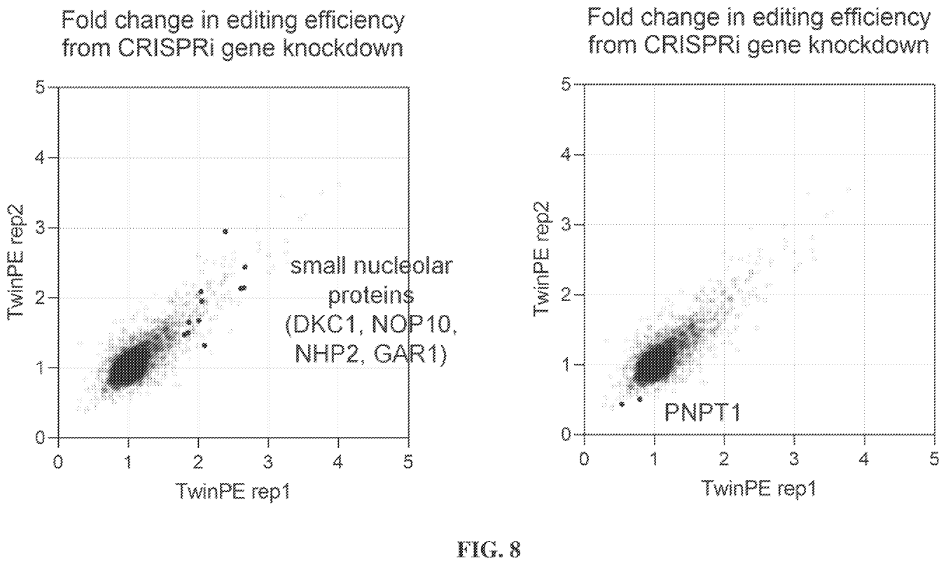

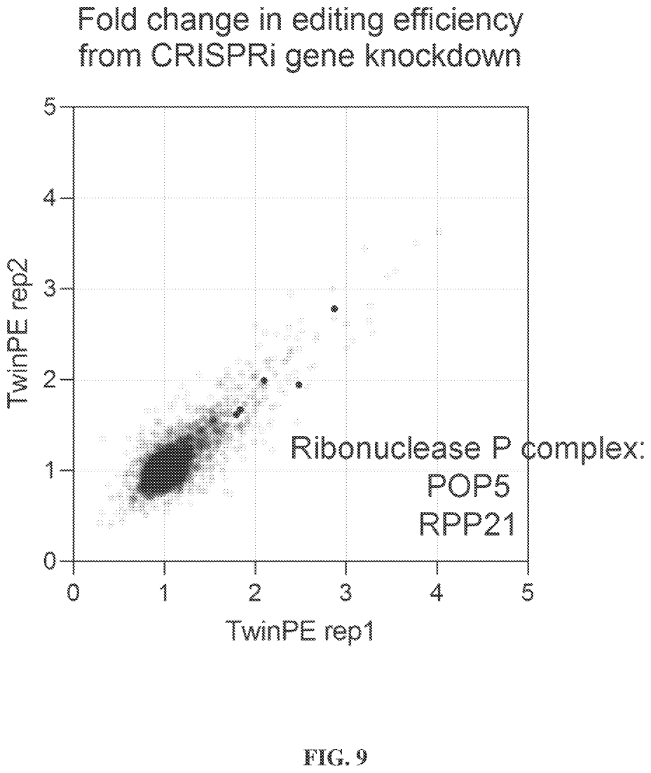

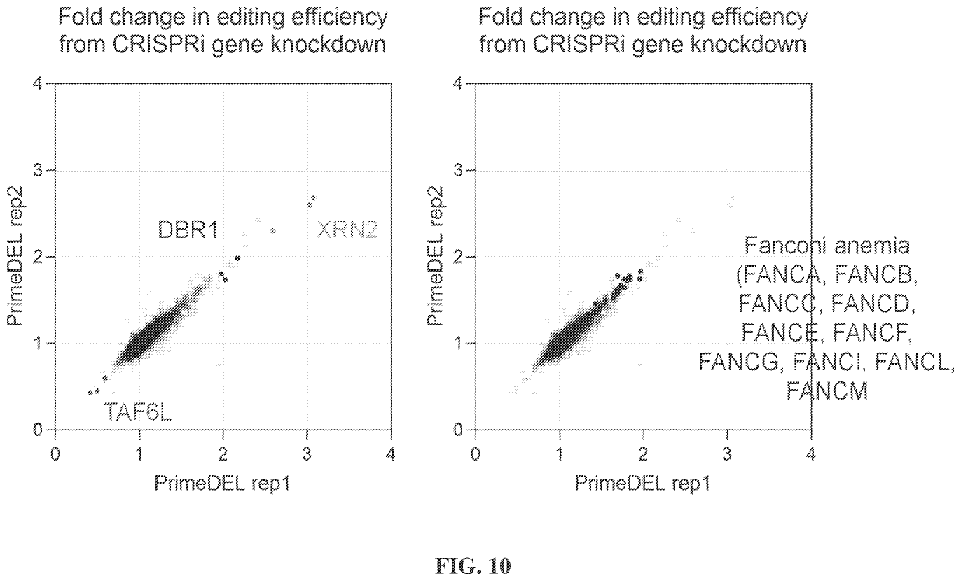

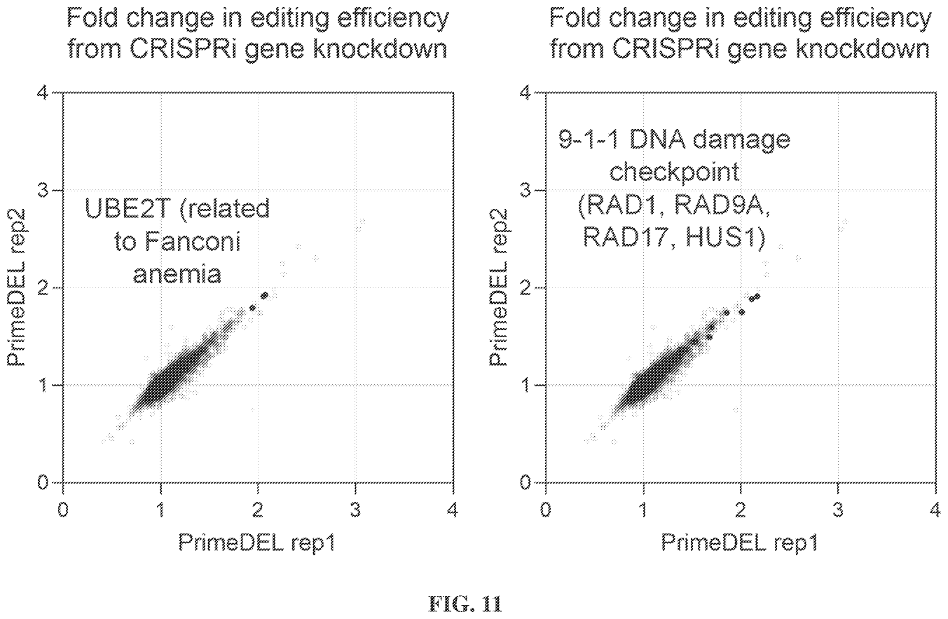

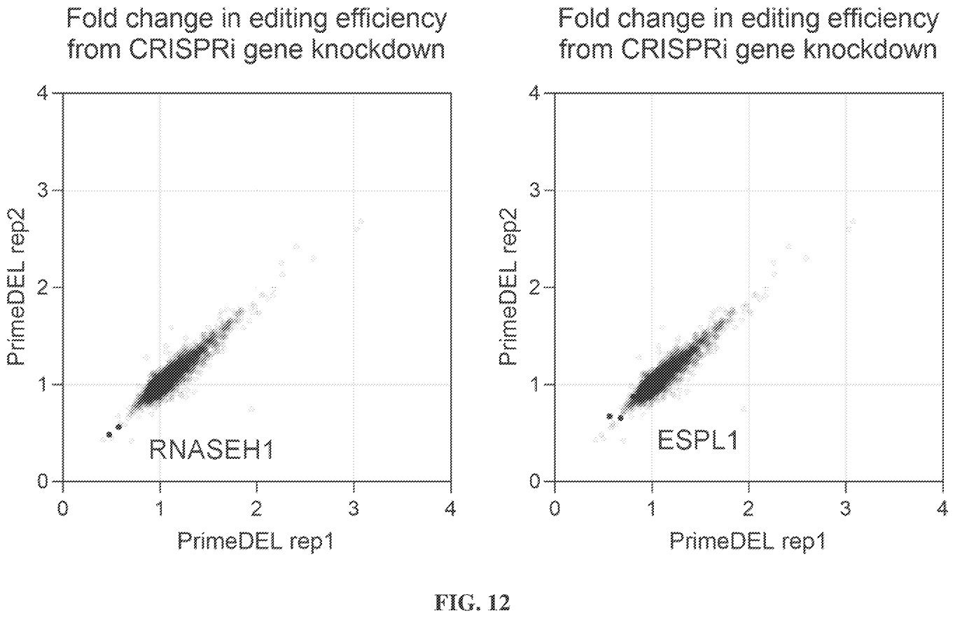

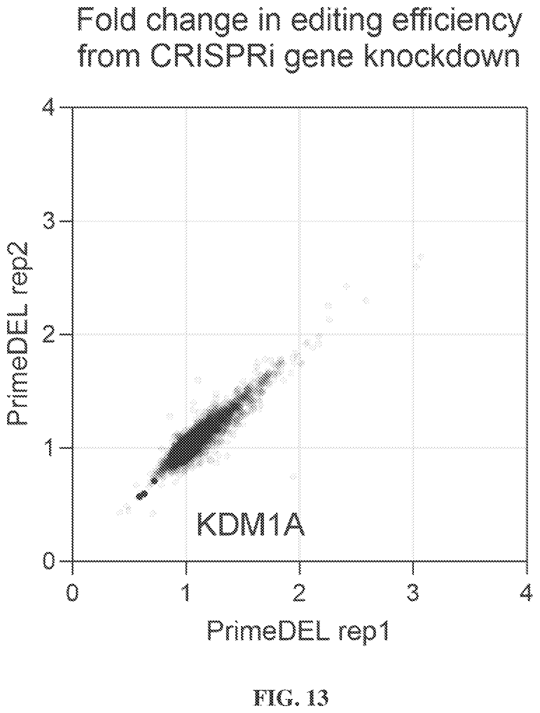

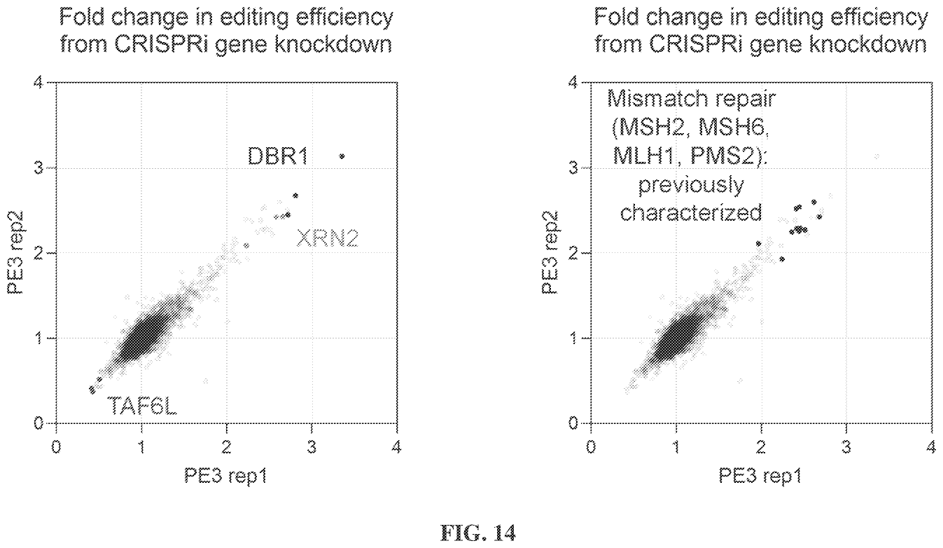

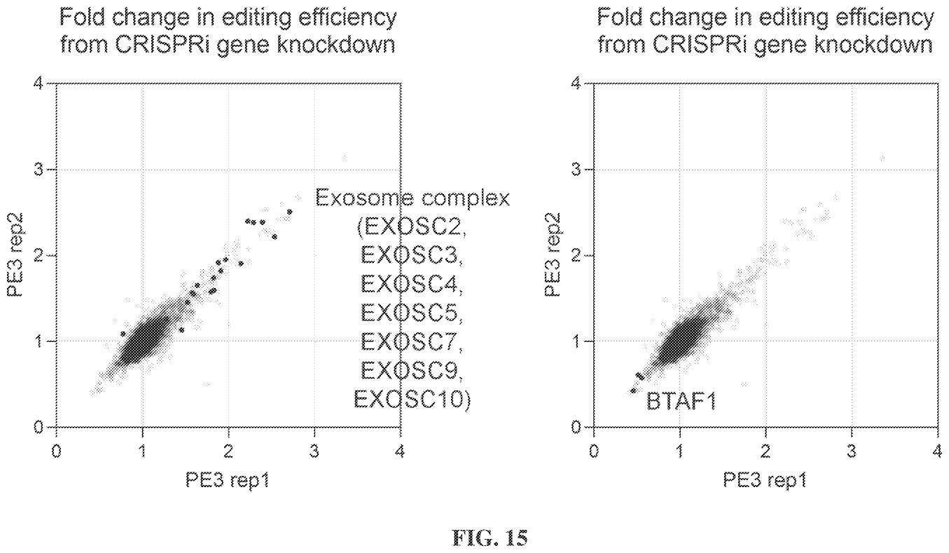

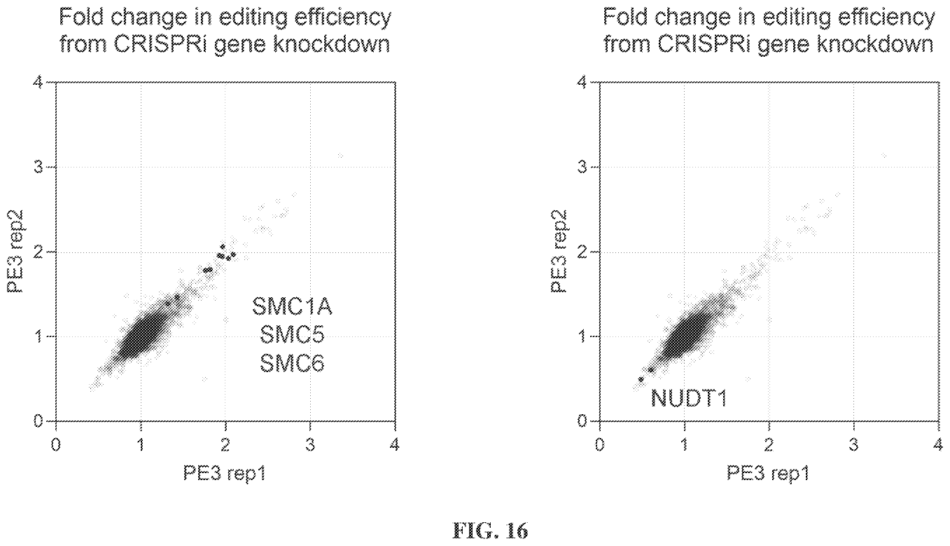

All prime editing CRISPRi screens were performed in biological duplicate in K562 cells with integrated dCas9-BFP-KRAB (Addgene #46911) 3. Results of these screens are shown in FIGS. 5-16. K562 CRISPRi cells expressing dCas9-KRAB were transduced with the lentiviral library at 0.1 MOI (10% BFP+) in RPMI supplemented with 10% FBS, 100 U mL⁻¹ penicillin, 100 μg mL⁻¹ streptomycin, 292 μg mL⁻¹ L-Glutamine, and 8 μg mL⁻¹ polybrene (Sigma-Aldrich) by centrifugation at 1000 g for 2 h at 33° C. 2 days post infection, K562 CRISPRi cells were treated with 3 μg mL⁻¹ puromycin (Thermo Fisher Scientific) to select for cells with integrated library members. After infection, the density of the K562 CRISPRi cells was maintained at approximately 5×10⁵ mL⁻¹ and the culture was replaced by fresh RPMI supplemented with 10% FBS, 100 U mL⁻¹ penicillin, 100 μg mL⁻¹ streptomycin, 292 μg mL⁻¹ L-Glutamine, and 3 μg mL⁻¹ puromycin 3 days and 5 days post infection. During media replacement, the cells were pelleted, washed with DPBS and resuspended in fresh media to remove dead cells. All centrifugations were performed at 200 g for 5 min in 50 mL canonical tubes to avoid cell loss. 6 days post infection, the culture was replaced twice by fresh RPMI supplemented with 10% FBS and 292 μg mL⁻¹ L-Glutamine (Thermo Fisher Scientific) to remove dead cells and antibiotics. Throughout lentiviral transduction and selection steps, cells were analyzed for BFP fluorescence on a flow cytometer to ensure completed selection. 7 days post infection, the cells were electroporated using the SE Cell Line 4D-Nucleofector X kit L (Lonza) with 1×10⁷ cells (program FF-120) and the following plasmid amounts for each condition: for PE3, 2 μg SaPEmax-P2A-BlastR plasmid, 0.5 μg Sa-pegRNA plasmid for installing a +6 G·C to C·G edit at the pre-validated edit site, and 0.2 μg Sa-sgRNA plasmid for +50 complementary-strand nicking was transfected; for twin prime editing, 12.5 μg SaPEmax-P2A-BlastR plasmid and 2.5 μg of each Sa-pegRNA plasmid for replacing 50 bp with the 38-bp attB sequence for Bxb1 recombination was transfected; for PRIME-Del editing, 5 μg SaPEmax-P2A-BlastR plasmid and 1.25 μg of each Sa-pegRNA plasmid for deleting 50 bp between the protospacers was transfected. After electroporation, the cells were seeded at a density of 5×10⁵ mL⁻¹ in RPMI supplemented with 10% FBS and 292 μg mL⁻¹ L-Glutamine. 48 h post electroporation, the cultures were pipetted up and down 5 times to prevent cells from clumping. 24 h post electroporation, 10 ng/μl blasticidin was added to the media to begin antibiotic selection for SaPEmax-expressing cells. 96 h post electroporation, the cells were pelleted at 1000 g for 10 min, washed with DPBS, pelleted at 1000 g for 10 min, and then stored at −80° C.

### High-Throughput Sequencing of CRISPRi Prime Editing Libraries

Genomic DNA was extracted from all CRISPRi prime editing screen cells using NucleoSpin Blood XL Maxi kit (Machery-Nagel). The entirety of the genomic DNA from each screen condition was used in the initial round of PCR (PCR1) to amplify the 453-bp region containing CRISPRi sgRNA and edit site. Each 100 μL PCR1 reaction was performed with 4 μg of genomic DNA as template, 1 μM of each primer for amplifying pPC1655 sgRNA and edit site, and 50 μL of NEBNext Ultra II Q5 Master Mix (New England BioLabs) on a BioRad C1000 thermal cycler with the following thermocycler conditions: 98° C. for 30 s, 22 cycles of [98° C. for 10 s, 65° C. for 75 s], followed by 65° C. for 5 min. These amplification reactions were verified by agarose gel electrophoresis and ethidium bromide staining. 1 mL of PCR1 product from each test condition and 1.5 mL of PCR1 product from each control condition were purified with 0.5× right-side and 0.9× left-side SPRIselect (Beckman Coulter). A following PCR step (PCR2) enabled indexing of the samples by the addition of i7 and i5 Illumina barcodes, and 200 μl PCR2 reactions were performed for each screen condition. For each PCR2 reaction, 20 ng of PCR1 amplicon was used as template along with 100 μL of KAPA HiFi HotStart ReadyMix (Roche Molecular Systems) and 600 nM of each barcoding primer with the following thermocycler conditions: 98° C. for 2 min, 8 cycles of [98° C. for 15 s, 61° C. for 20 s, 72° C. for 40 s], followed by 72° C. for 2 min. The reactions were verified by agarose gel electrophoresis and ethidium bromide staining. PCR2 products from four PCR2 products were purified using 0.75× left-side SPRIselect and quantified on an Agilent 4200 Bioanalyzer prior to pooling. Libraries were sequenced with the NovaSeq 6000 S1 Reagent Kit v1.5 (Illumina) with two 8-nt index reads, 45 cycles for R1 read, 265 cycles for R2 read.

### Processing of CRISPRi Prime Editing Screen Data

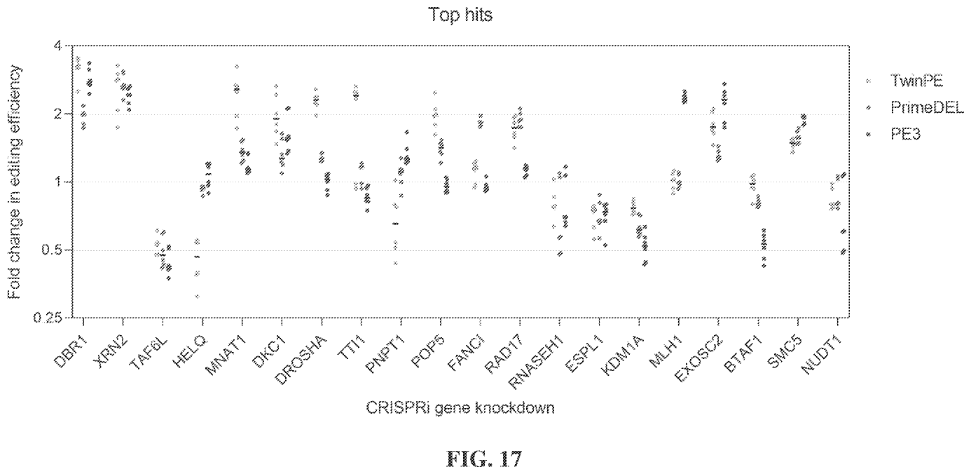

Sequencing data from the NovaSeq were demultiplexed into individual screens based on index reads. R1 reads specified the sequence of the CRISPRi sgRNA that programs the CRISPRi gene perturbation, while R2 reads contained the sequence outcome from PE3, TwinPE, or PRIME-Del editing. Within each screen, R2 reads were demultiplexed into individual fastq read files based on the CRISPRi sgRNA sequence in R1. Briefly, R1 reads were matched to the first 19 nt of the CRISPRi sgRNA sequences in the library, allowing for a maximum of 1 mismatch. These individual R2 fastqs (each corresponding to all editing outcomes in cells with a given CRISPRi sgRNA) were then analyzed with CRISPResso2⁴ to obtain editing and indel frequencies for each CRISPRi perturbation. For CRISPResso analysis, R2 read sequences were aligned to a reference sequence in HDR mode using the parameters “−q 30”. For each amplicon, the CRISPResso2 quantification window was positioned to include the entire sequence between Sa-pegRNA protospacers, including the full protospacer sequences. CRISPResso2 was run in HDR mode using the intended editing outcome as the expected allele (−e). Frequencies for these edits were quantified as: (number of HDR-aligned reads)/(number of reference-aligned reads). All indel frequencies were quantified as: (number of indel-containing reads)/(number of reference-aligned reads). Results of these screens are shown in Tables X3-X8. Top hits and modulators are summarized in FIG. 17 and Tables X1-X2.

### References in Example 1

1. Chen, P. J. et al. Enhanced prime editing systems by manipulating cellular determinants of editing outcomes. Cell 184, 5635-5652.e5629, doi: 10.1016/j.cell.2021.09.018 (2021).
2. Hussmann, J. A. et al. Mapping the genetic landscape of DNA double-strand break repair. Cell 184, 5653-5669.e5625, doi: 10.1016/j.cell.2021.10.002 (2021).
3. Gilbert, Luke A. et al. Genome-Scale CRISPR-Mediated Control of Gene Repression and Activation. Cell 159, 647-661, doi: 10.1016/j.cell.2014.09.029 (2014).
4. Clement, K. et al. CRISPResso2 provides accurate and rapid genome editing sequence analysis. Nature Biotechnology 37, 224-226, doi: 10.1038/s41587-019-0032-3 (2019).

### Example 2. Validation of TwinPE, PrimeDEL, PE2, and PE3 Edits

### TwinPE

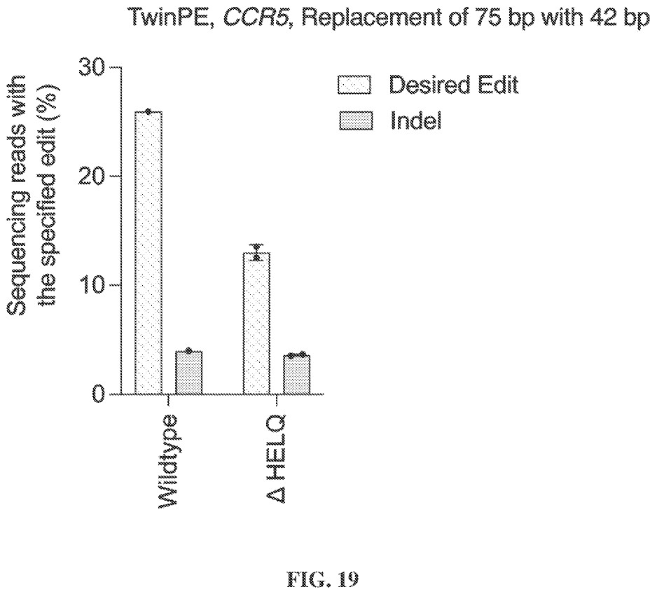

HELQ-knockout retinal pigment epithelium (RPE) cells were created by Cas9 nuclease and a pair of sgRNA to remove most conserved exons of the hit candidate. The edited cells were then sorted to single cell clones, re-populated, and sequenced to confirm knockout effects. Homozygous clones were treated with TwinPE strategy (WT RPE cells treated similarly by TwinPE as the control). Editing efficiency was quantified by Miseq analysis. As shown in FIG. 19, knockout of HELQ inhibited the TwinPE editing efficiency by ~2-fold, confirming HELQ identified from the CRISPRi screen.

### PrimeDEL

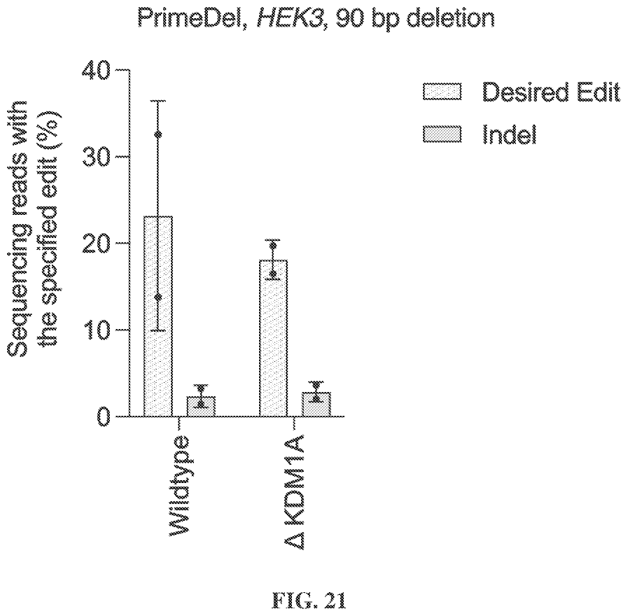

KDM1A-knockout retinal pigment epithelium (RPE) cells were created by Cas9 nuclease and a pair of sgRNA to remove most conserved exons of the hit candidate. The edited cells were then sorted to single cell clones, re-populated, and sequenced to confirm knockout effects. Homozygous clones were treated with PrimeDEL (WT RPE cells treated similarly by PrimeDEL as the control). Editing efficiency was quantified by Miseq analysis. As shown in FIG. 21, knockout of KDM1A impaired PrimeDEL editing efficiency by 1.3-fold compared WT, confirming KDM1A's effect on PrimeDEL identified from the CRISPRi screen.

### PE2

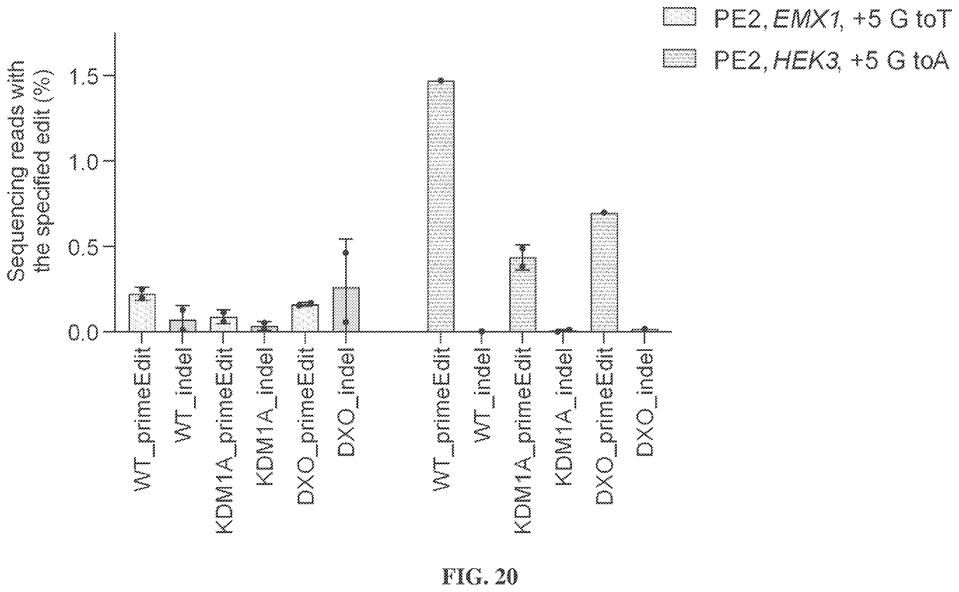

KDM1A- and DXO-knockout retinal pigment epithelium (RPE) cells were created by Cas9 nuclease and a pair of sgRNA to remove most conserved exons of the hit candidate. The edited cells were then sorted to single cell clones, re-populated, and sequenced to confirm knockout effects. Homozygous clones were treated with PE2 (WT RPE cells treated similarly by PE2 as the control). Editing efficiency was quantified by Miseq analysis. As shown in FIG. 20, knockout of KDM1A and DXO impaired PE3 editing efficiency up to 2-fold compared to wildtype cells, confirming these two hits identified from the CRISPRi screen.

### PE3

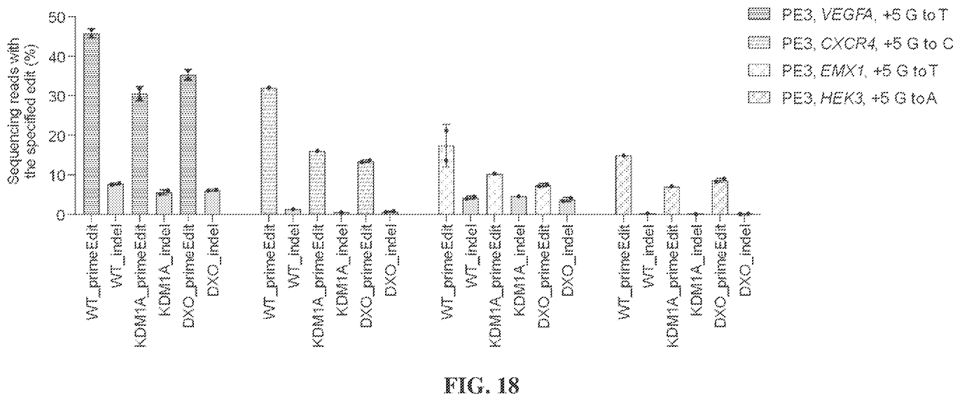

KDM1A- and DXO-knockout retinal pigment epithelium (RPE) cells were created by Cas9 nuclease and a pair of sgRNA to remove the most conserved exons of the hit candidate. The edited cells were then FACS-sorted to single cell clone on 96-well plate, re-populated, and sequenced to confirm knockout effects. Homozygous clones were then expanded, replated, and transfected with PE3 (WT RPE cells treated similarly by PE3 as the control) using Lipofectamine2000 per manufacture's protocol. Genomic DNA was harvested, PCR-amplified, and sequenced by miseq. CRISPResso2 analysis was performed to analyze the editing efficiency. As shown in FIG. 18, knockout of KDM1A and DXO impaired PE3 editing efficiency by 1.3-2.5-fold compared to WT cells, confirming these two hits identified from the CRISPRi screen.

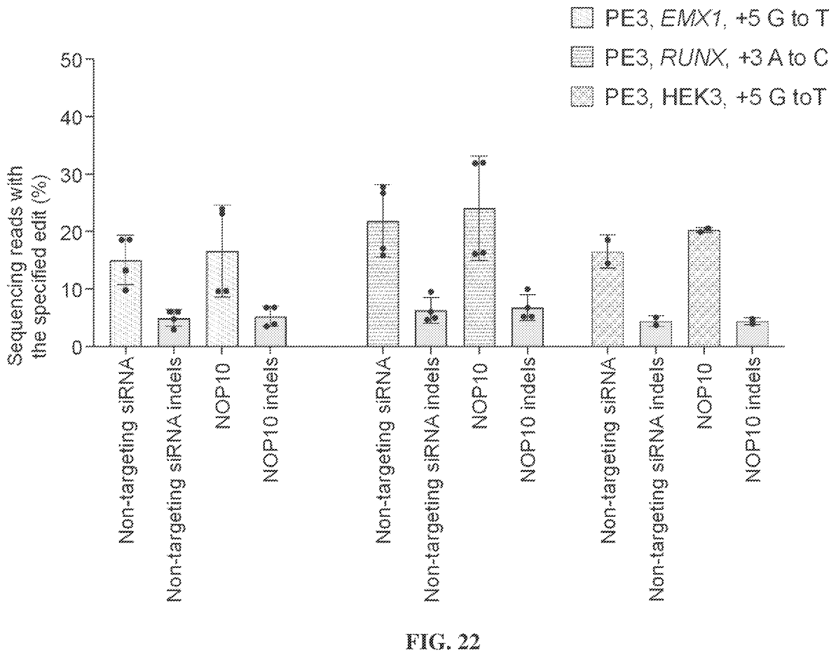

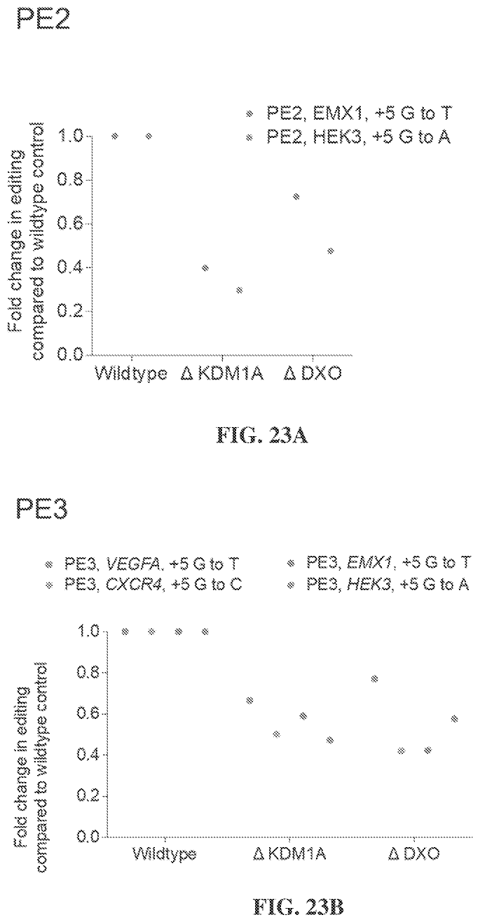

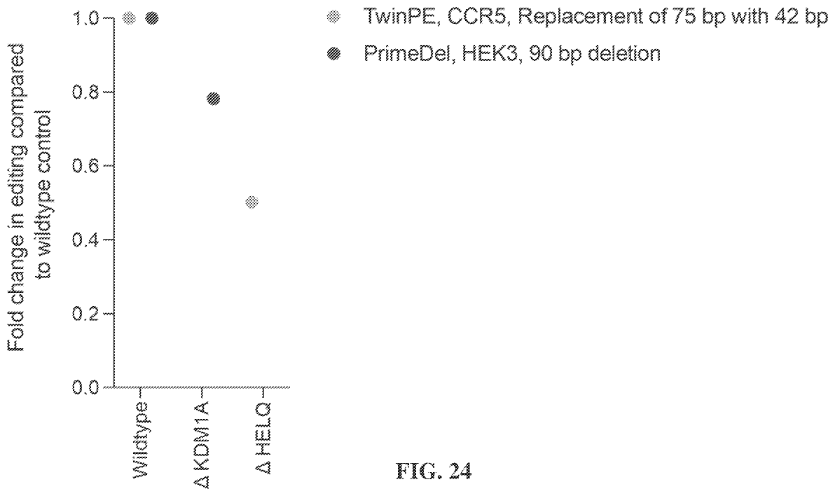

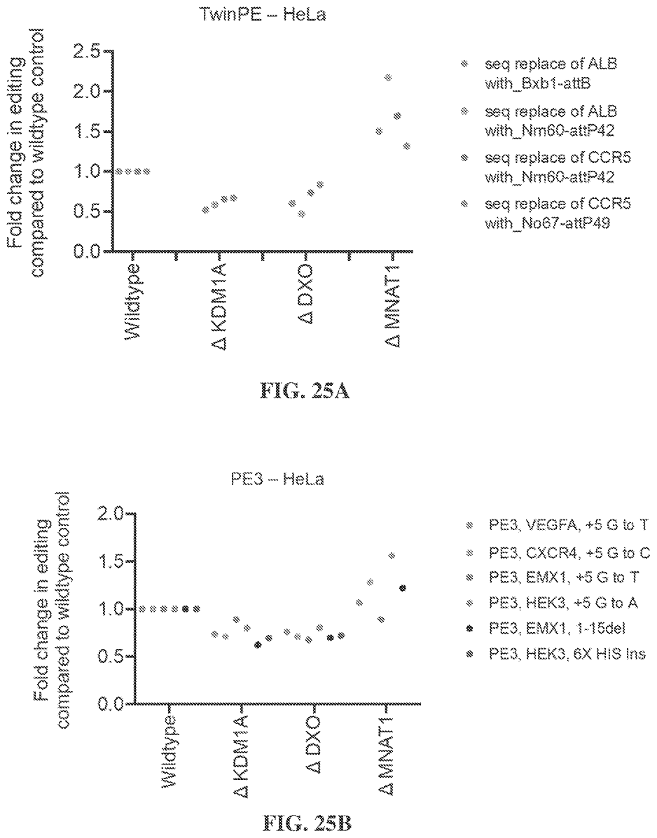

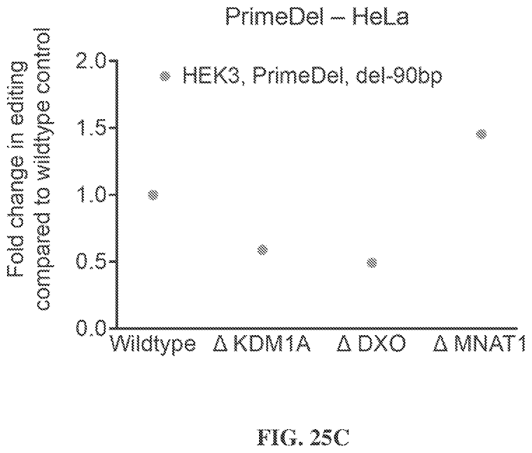

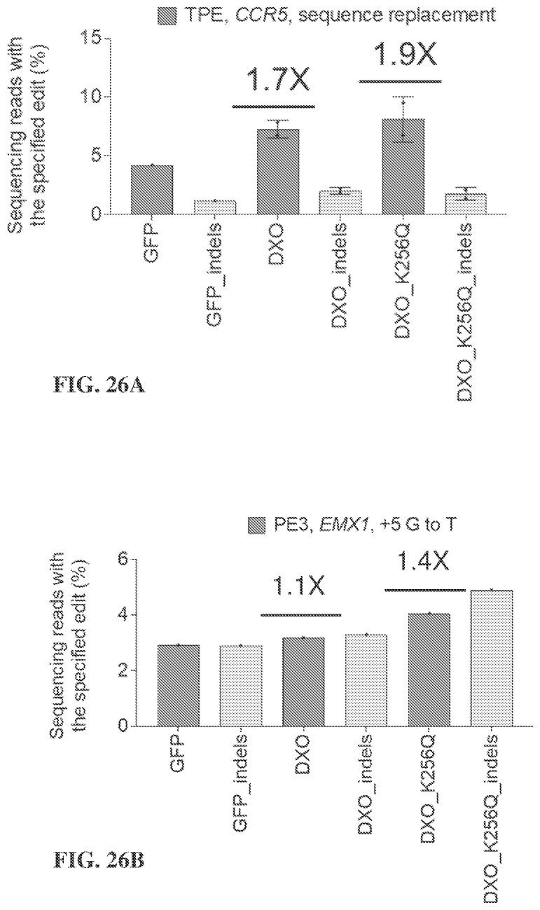

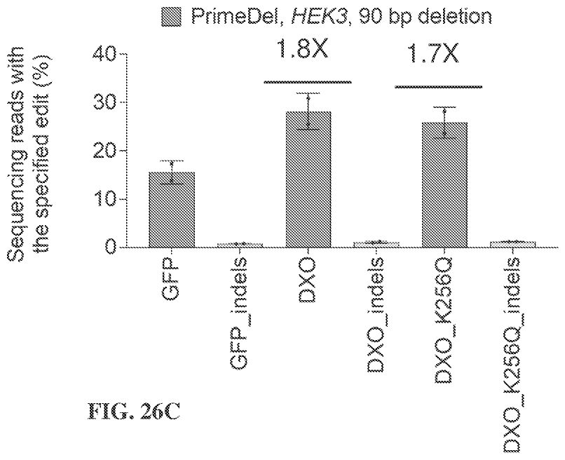

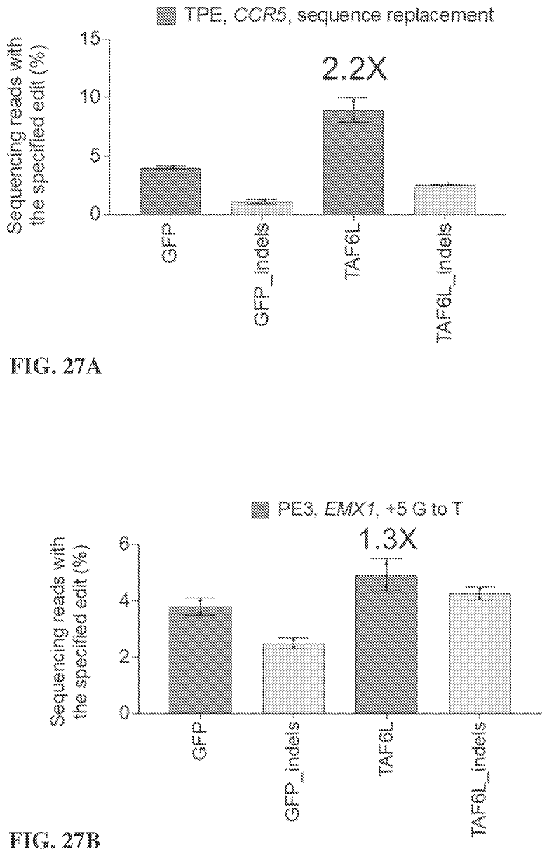

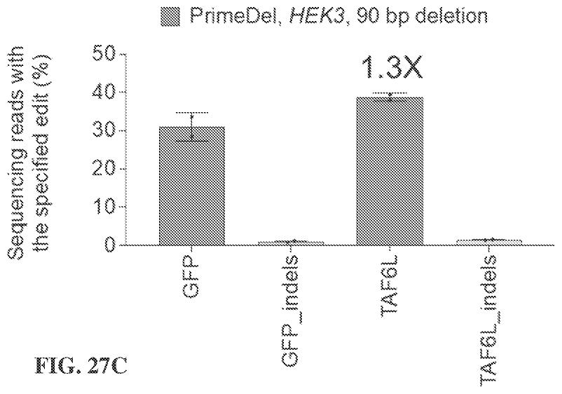

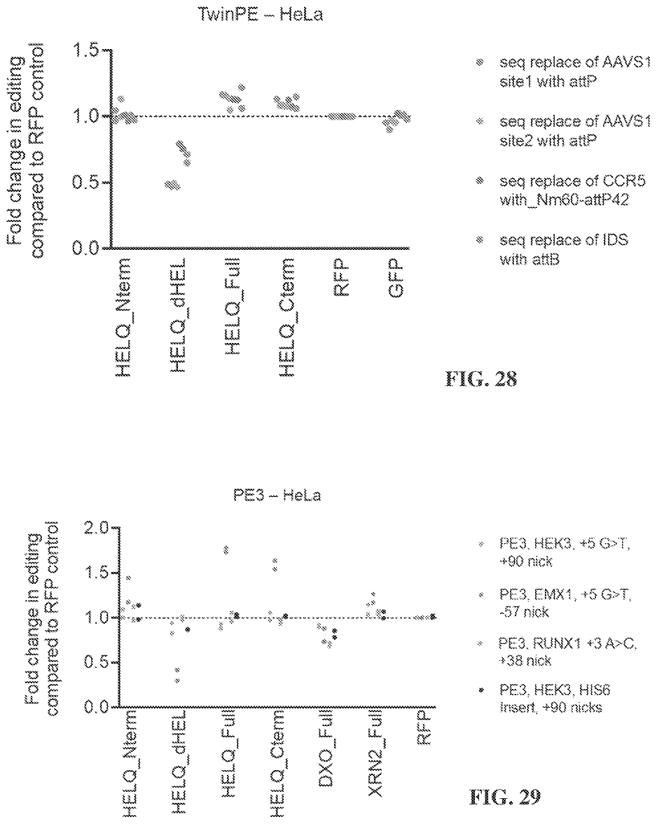

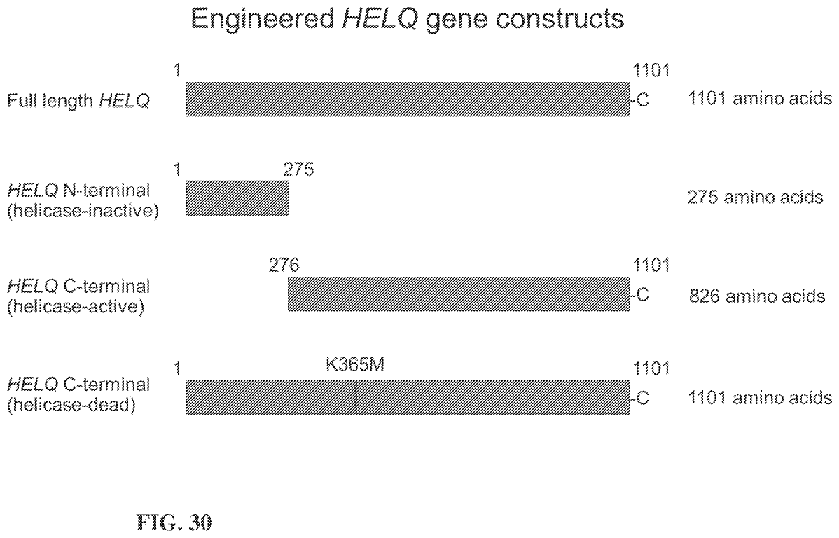

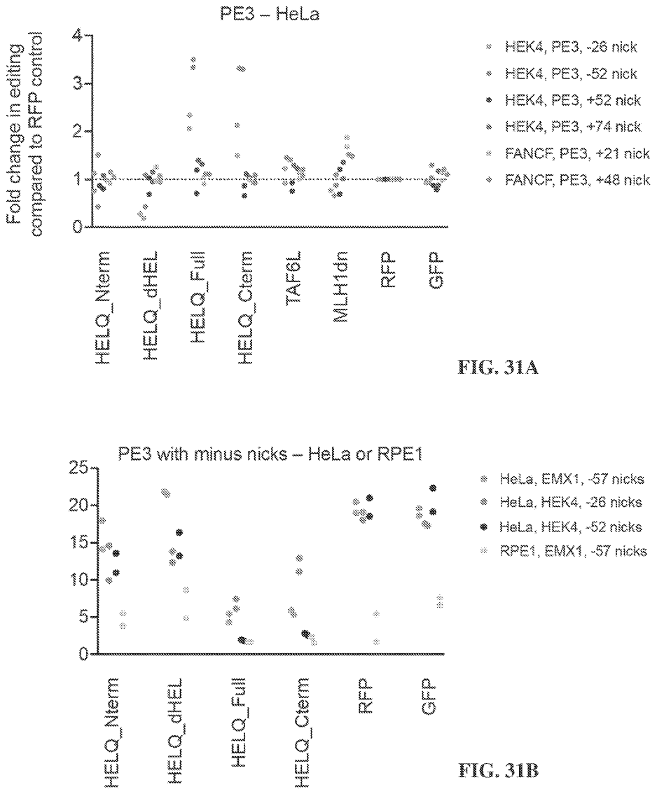

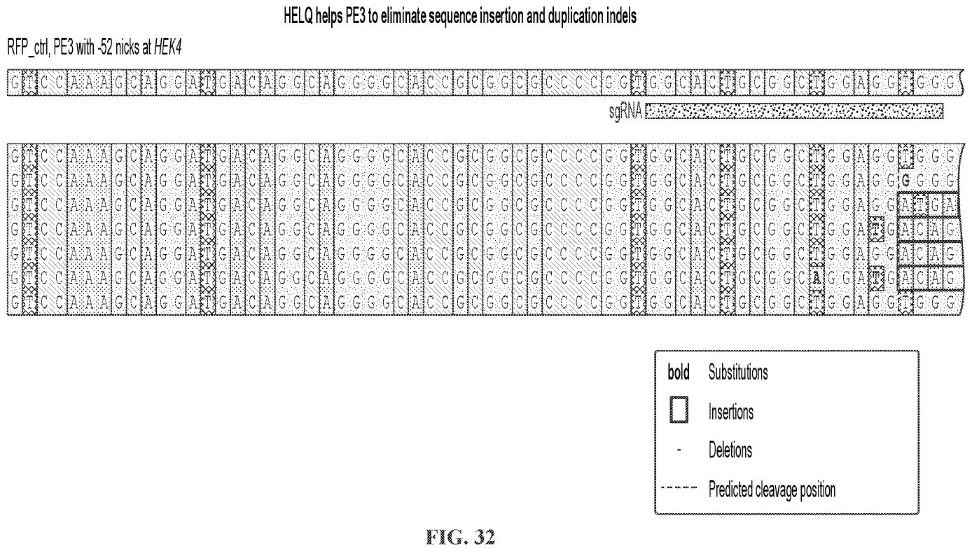

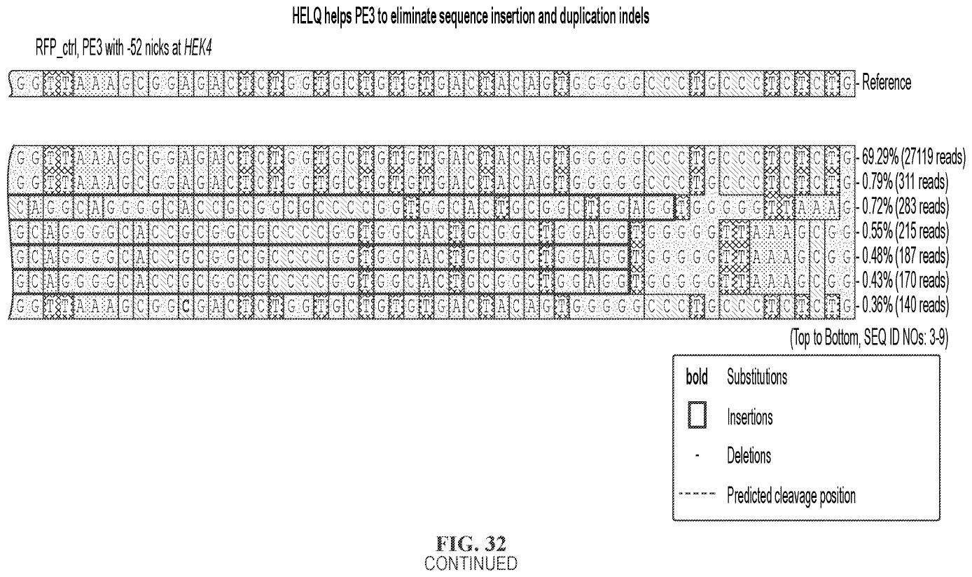

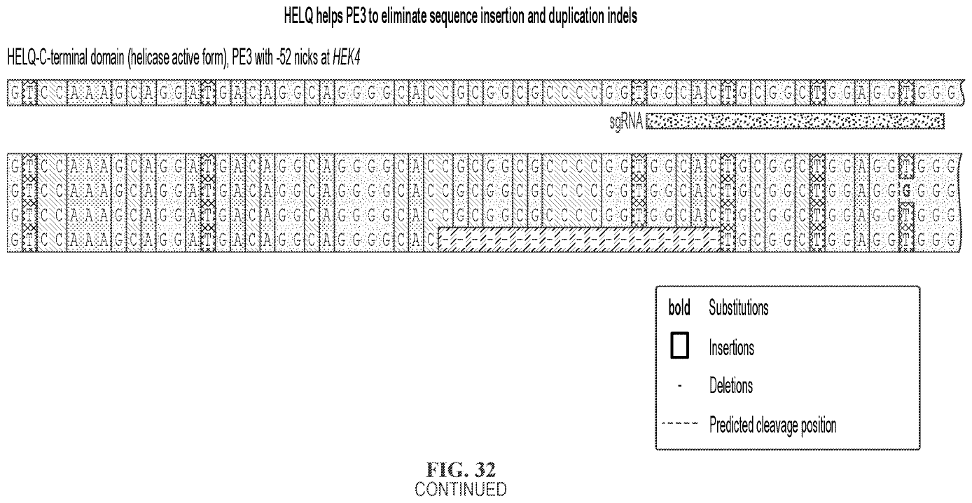

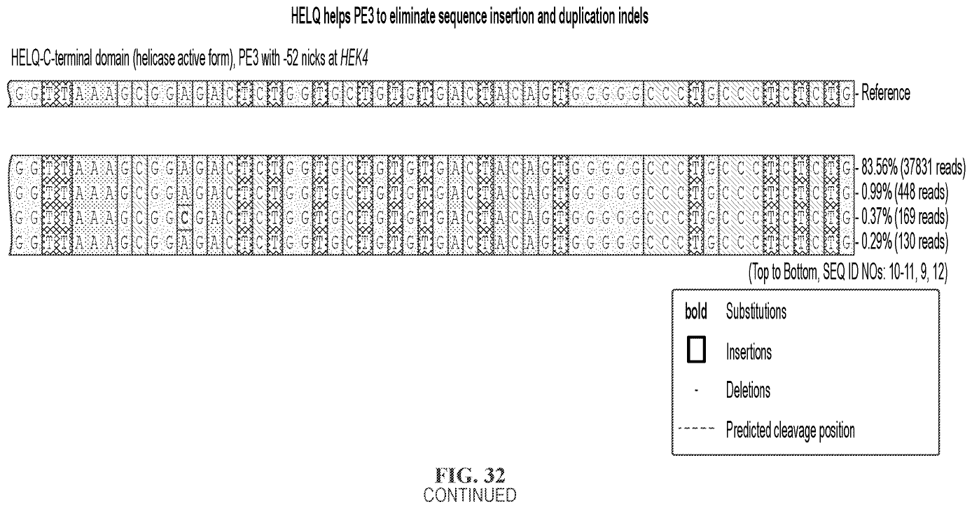

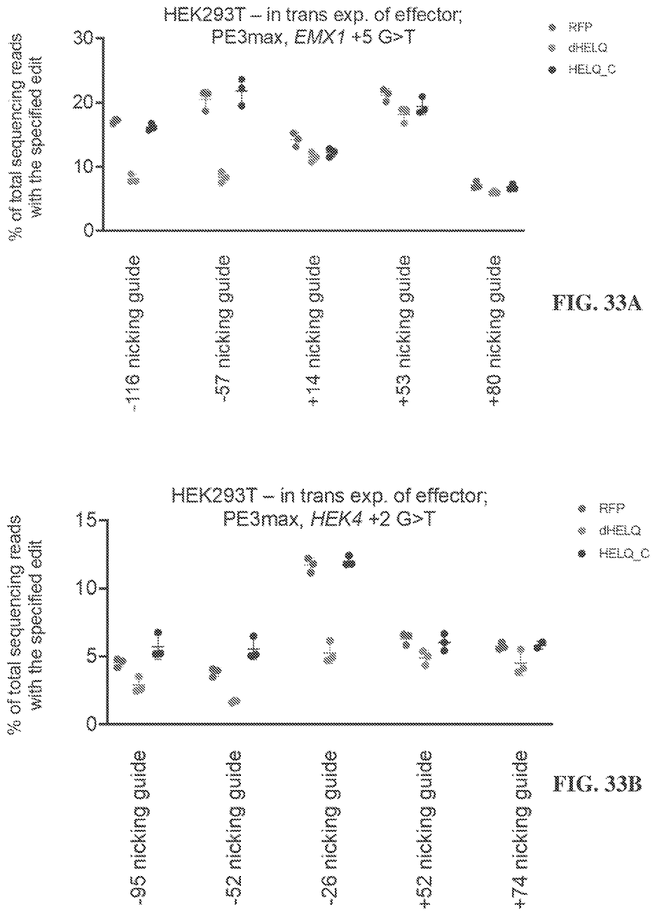

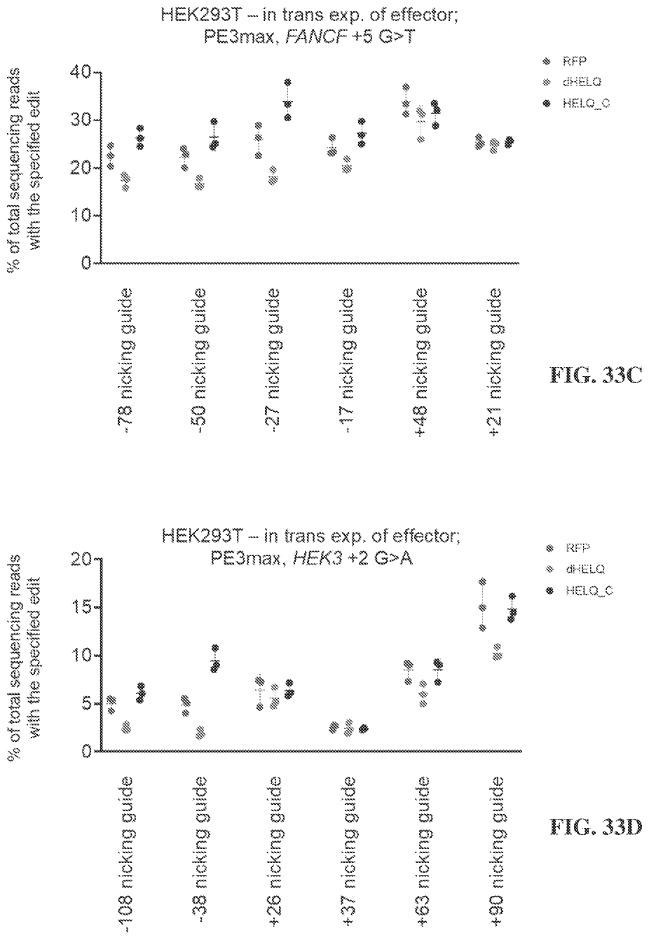

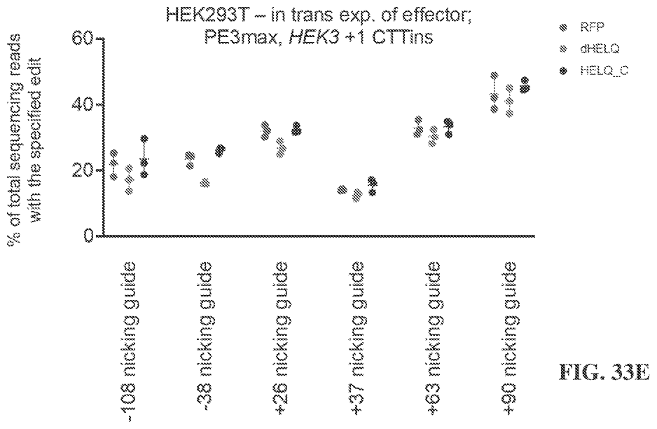

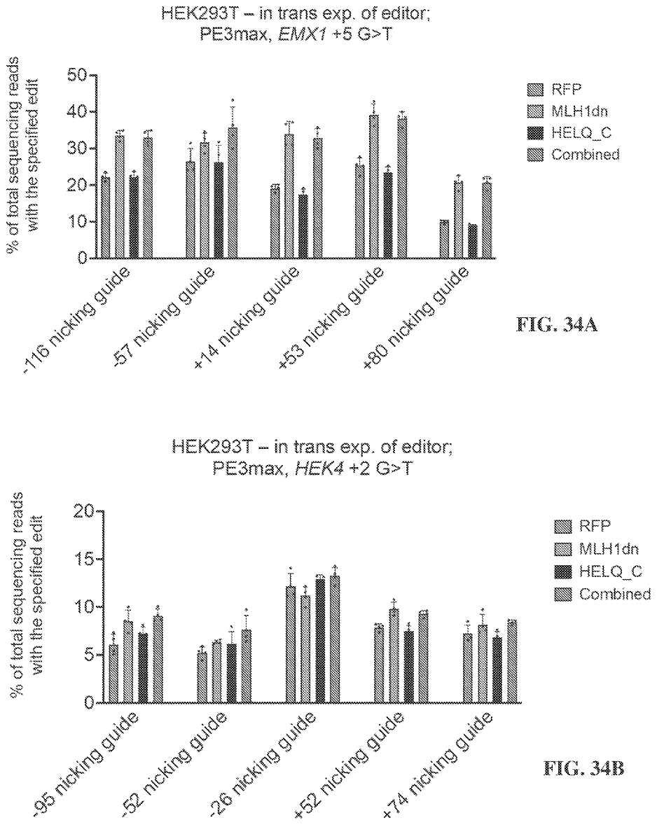

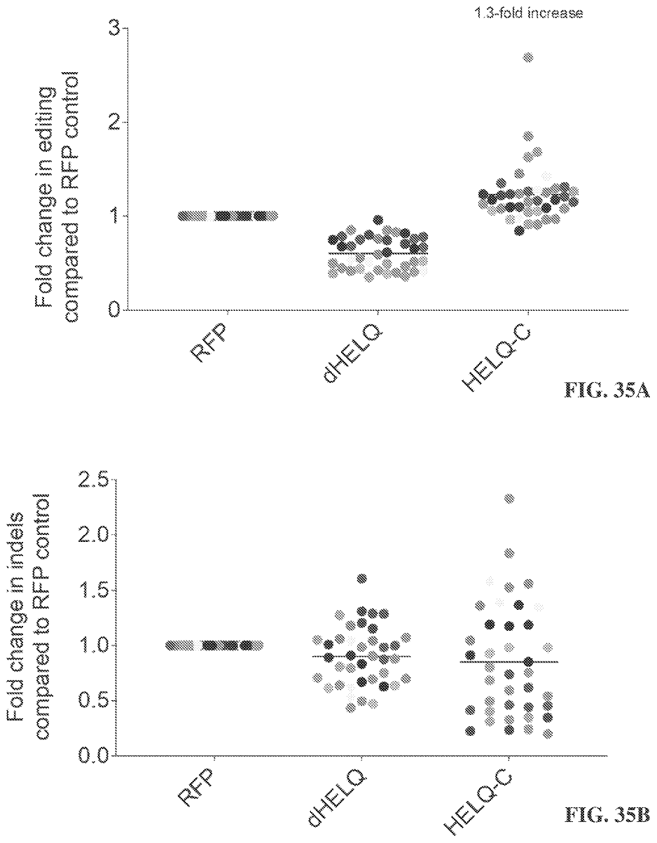

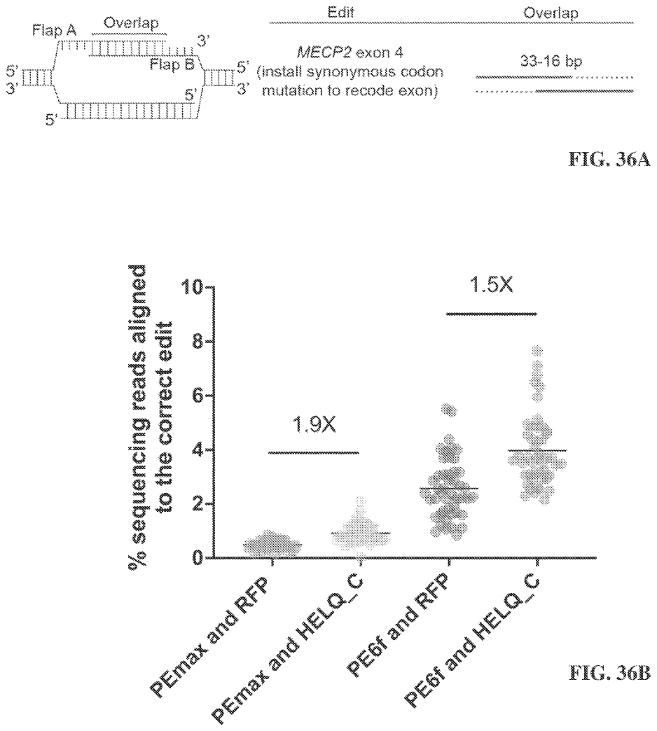

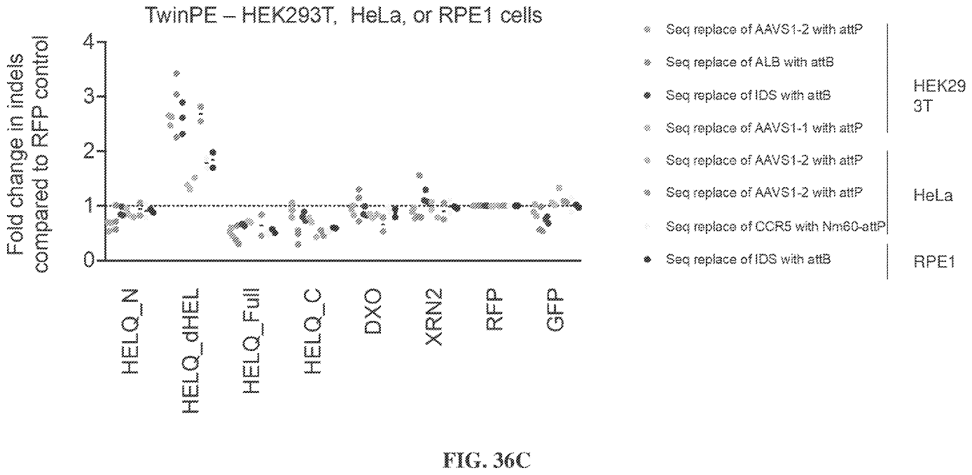

Additionally, PE3 was tested in HEK293T cells. HEK293T cells were seeded on 6-well plates (Corning) at 7.5×10\hat{ }5 cells per well in DMEM plus GlutaMAX supplemented with 10% FBS. At 60% confluency 16 h after seeding, cells were transfected with 9 mL Lipofectamine RNAiMAX (Thermo Fisher Scientific) according to the manufacturer's protocol and 90 μmol ON-TARGETplus SMARTpool siRNAs (Horizon Discovery). One day after transfection, media was replaced with fresh DMEM plus GlutaMAX supplemented with 10% FBS. 2 days after transfection, cells were washed once with PBS and resuspended using TrypLE (Thermo Fisher Scientific) and DMEM plus GlutaMAX supplemented with 10% FBS. HEK293T cells were then seeded on 96-well plates (Corning) at 2.5×10\hat{ }4 cells per well. Between 16 and 24 h after seeding, cells were transfected at 60%-80% confluency with 0.5 mL Lipofectamine 2000 (Thermo Fisher Scientific) according to the manufacturer's protocol and 200 ng prime editor plasmid, 66 ng pegRNA plasmid, 22 ng sgRNA plasmid, and 5 μmol of the same ON-TARGETplus SMARTpool siRNAs used in the first transfection. For control conditions, cells were treated with non-targeting siRNAs in both transfections. Cells were cultured for 72 h after the second transfection before genomic DNA extraction. Genomic DNA were then subjected to PCR and miseq analysis. Editing efficiency by PE3 is shown in FIG. 22, suggesting knockdown of NOP10 improves PE3 editing efficiency by ~1.3-fold compared to non-targeting siRNA treated cells.

### Tables Referenced in Examples

Table X1 shows a summary of the top hits that were identified from the CRISPRi screen described herein. The range of fold-change on percentage of editing efficiency upon knockdown of the gene are shown in the table. PE configurations are also annotated in the table indicating knockdown of the genes in this table can have an effect on multiple PE configurations. The following subunits were identified from the same complex or pathway: all four components, DKC1, NOP10, NHP2, GAR1, from H/ACA complex; SMC5 and SMC6 from SMC5-SMC6 complex; RAD9A and RAD17 from 9-1-1 complex; and EXOSC2 and EXOSC5 from RNA exosome complex.

TABLE X1
Genetic modulators conserved for multiple PE configurations

| | | CRISPRi- | |
|---|---|---|---|
| | | KD effect on | |
| Gene | Gene/Pathway function | % editing | PE configurations |
| DBR1 | Cleaves 2′-5′ phosphodiester linkage at the | 2.0-3.5X | TPE, PD, PE3 |
| | branch point of lariat intron pre-mRNAs | increase | |
| XRN2 | 5′-3′ exoribonuclease that promotes termination | 2.0-3.5X | TPE, PD, PE3 |
| | of RNAPII transcription | increase | |
| DKC1 | A member of H/ACA small nucleolar | 1.4-2.2X | TPE, PD, PE3 |
| | ribonucleoprotein that involves in rRNA | increase | |
| | processing and modifications | | |
| NOP10 | A member of H/ACA small nucleolar | 1.5-2.4X | TPE, PD, PE3 |
| | ribonucleoprotein that involves in rRNA | increase | |
| | processing and modifications | | |
| NHP2 | A member of H/ACA small nucleolar | 1.4-2.0X | TPE, PD, PE3 |
| | ribonucleoprotein that involves in rRNA | increase | |
| | processing and modifications | | |
| GAR1 | A member of H/ACA small nucleolar | 1.4-1.8X | TPE, PD, PE3 |
| | ribonucleoprotein that involves in rRNA | increase | |
| | processing and modifications | | |
| SMC5 | Core component of SMC5-SMC6 complex that | 1.5-1.9X | TPE, PD, PE3 |
| | involves in DNA DSBs by homologous | increase | |
| | recombination | | |
| SMC6 | Core component of SMC5-SMC6 complex that | 1.6-1.9X | TPE, PD, PE3 |
| | involves in DNA DSBs by homologous | increase | |
| | recombination | | |
| TAF6L | Function as a component of the PCAF complex | 2.0-2.3X | TPE, PD, PE3 |
| | to acetylate histones in a nucleosomal context | decrease | |
| ESPL1 | Cleavage of the cohesin subunit RAD21 during | 1.5X-1.6X | TPE, PD, PE3 |
| | separation of sister chromatids | decrease | |
| RAD9A | Component of the 9-1-1 cell-cycle checkpoint | 1.6X-1.7X | TPE and PD |
| | response complex that plays a major role in DNA | increase | |
| | repair | | |
| RAD17 | Component of the 9-1-1 cell-cycle checkpoint | 1.7X-1.9X | TPE and PD |
| | response complex that plays a major role in DNA | increase | |
| | repair | | |
| CCNH | Subunit of the CDK-activating kinase (CAK) | 1.5X-2.6X | TPE and PD1 |
| | complex that activates TFIIH by phosphorylation | increase | |
| | for RNA pol II transcription | | |
| EXOSC2 | Subunit of RNA exosome complex that functions | 1.9X-2.3X | TPE and PE3 |
| | in RNA processing (3′->5′ exoribonuclease) and | increase | |
| | small RNA maturation | | |
| EXOSC5 | Subunit of RNA exosome complex that functions | 1.7X-2.1X | TPE and PE3 |
| | in RNA processing (3′->5′ exoribonuclease) and | increase | |
| | small RNA maturation | | |
| SMC1A | Involves in chromosome cohesion during cell | 1.5X-1.6X | TPE and PE3 |
| | cycle and in DNA repair | increase | |
| KDM1A | A histone demethylase that can demethylate both | 1.6X-2.0X | PD and PE3 |
| | HEK4me and HEK9me of histone H3 | decrease | |

Table X2 shows a summary of top hits that were identified from the CRISPRi screen. The fold-change on percentage of editing efficiency upon knockdown of the gene are shown in the table. The list of gene candidates in this table suggests each affects one specific PE configuration. The following subunits were identified from the same complex or pathway: GTF2H1, GTF2H4, GTF2F2, MNAT1, CCNH in Pol II transcription pathway; and RPP21 and POP5 from ribonuclease P complex.

TABLE X2
Unique genetic modulators for specific PE configurations

| | | CRISPRi-KD | PE |
|---|---|---|---|
| | | effect on % | configura- |
| Gene | Gene/Pathway function | editing | tions |
| MNAT1 | Stabilizes the cyclin H-CDK7 complex to form | 2.5X increase | TwinPE |
| | a functional CDK-activating kinase (CAK) | | |
| | enzymatic complex | | |
| GTF2H1 | Component of TFIIH that involves in | 2.8X increase | TwinPE |
| | transcription coupled DNA repair and RNA | | |
| | polymerase II transcription | | |
| GTF2H4 | Component of TFIIH that involves in | 2.6X increase | TwinPE |
| | transcription coupled DNA repair and RNA | | |
| | polymerase II transcription | | |
| RPP21 | Component of ribonuclease P, a | 2.3X increase | TwinPE |
| | ribonucleoprotein complex that generates | | |
| | mature tRNA molecules by cleaving their 5′- | | |
| | ends | | |
| POP5 | Component of ribonuclease P, a | 2.1X increase | TwinPE |
| | ribonucleoprotein complex that generates | | |
| | mature tRNA molecules by cleaving their 5′- | | |
| | ends | | |
| GTF2F2 | A transcription initiation factor that binds to | 2.4X increase | TwinPE |
| | RNA polymerase II and helps to recruit it to the | | |
| | initiation complex in collaboration with TFIIB | | |
| TTI1 | Regulator of the DNA damage response; part of | 2.6X increase | TwinPE |
| | the TTT complex that is required to stabilize | | |
| | protein | | |
| DDX20 | Functions as putative RNA helicases in altering | 2.4X increase | TwinPE |
| | RNA secondary structure | | |
| DROSHA | Ribonuclease III double-stranded RNA-specific | 2.3X increase | TwinPE |
| | endoribonuclease that is involved in the initial | | |
| | step of microRNA (miRNA) biogenesis. | | |
| PNPT1 | RNA-binding protein implicated in RNA | 2.1X decrease | TwinPE |
| | metabolic processes; catalyzation of single- | | |
| | stranded polyribonucleotides in the 3′-to-5′ | | |
| | direction | | |
| POLQ | DNA polymerase that promotes | 1.7X decrease | TwinPE |
| | microhomology-mediated end-joining | | |
| CCNB2 | Essential for the control of the cell cycle at the | 2.2X decrease | TwinPE |
| | G2/M (mitosis) transition | | |
| RPA2 | Part of the RPA complex that binds and | 1.8X decrease | TwinPE |
| | stabilizes single-stranded DNA intermediate | | |
| DPY30 | Part of the MLL1/MLL complex that involves in | 1.6X decrease | TwinPE |
| | the methylation of histone H3 at ‘Lys-4’, | | |
| | particularly trimethylation | | |
| HELQ | Single-stranded DNA-dependent ATPase and 5′ | 2.3X decrease | TwinPE |
| | to 3′ DNA helicase that involves in the repair of | | |
| | DNA cross-links and DSB resistance | | |
| DXO | Decap enzyme for NAD-capped RNAs | 2.0X decrease | PE3 |
| BTAF1 | Regulates transcription in association with | 2.0X decrease | PE3 |
| | TATA binding protein (TBP) | | |
| UBE2T | Acts as a specific E2 ubiquitin-conjugating | 2.0X increase | PRIME-Del |
| | enzyme for the Fanconi anemia complex | | |
| FANCI | Plays an essential role in the repair of DSB | 1.8X increase | PRIME-Del |
| | breaks and interstrand DNA cross-links by | | |
| | promoting FANCD2 monoubiquitination | | |
| FANCD2 | Required for maintenance of chromosomal | 1.8X increase | PRIME-Del |
| | stability | | |
| RNASEH1 | Endonuclease that specifically degrades the | 1.9X decrease | PRIME-Del |
| | RNA of RNA-DNA hybrids | | |

TABLE X3
Genes with increased editing efficiency
upon TwinPE CRISPRi knockdown

| | TwinPE (Approximate fold change, increased |
|---|---|
| Genes | editing upon CRISPRi KD) |
| CHAF1B | 3.73X |
| DBR1 | 3.34X |
| XRN2 | 2.98X |
| MNAT1 | 2.76X |
| CCNH | 2.73X |
| GTF2H4 | 2.63X |
| NOP10 | 2.53X |
| TTI1 | 2.49X |
| GTF2F2 | 2.43X |
| DROSHA | 2.34X |
| DDX20 | 2.32X |
| CDK7 | 2.32X |
| RPP21 | 2.29X |
| MYB | 2.25X |
| ERCC2 | 2.23X |
| DKC1 | 2.20X |
| CENPA | 2.18X |
| RPP40 | 2.18X |
| CHAF1A | 2.13X |
| POP5 | 2.13X |
| HINFP | 2.12X |
| GTF2H1 | 2.09X |
| XRCC5 | 2.08X |
| NHP2 | 2.02X |
| ERCC3 | 2.01X |
| RPP30 | 2.01X |
| INTS3 | 2.00X |
| HJURP | 1.99X |
| TAF1 | 1.95X |
| SMARCA5 | 1.94X |
| DICER1 | 1.93X |
| HNRNPC | 1.91X |
| EXOSC2 | 1.91X |
| POP1 | 1.91X |
| MTOR | 1.89X |
| XRCC6 | 1.89X |
| GAR1 | 1.88X |
| CENPP | 1.85X |
| RAD17 | 1.84X |
| RPP38 | 1.84X |
| TELO2 | 1.84X |
| BCCIP | 1.83X |
| RCL1 | 1.79X |
| NSMCE1 | 1.78X |
| CDK12 | 1.78X |
| POLR2I | 1.78X |
| GPN1 | 1.76X |
| SLBP | 1.76X |
| RPP14 | 1.75X |
| DIS3 | 1.75X |
| POP4 | 1.75X |
| POLR2H | 1.75X |
| TREX2 | 1.74X |
| EXOSC4 | 1.73X |
| EXOSC5 | 1.73X |
| DHX35 | 1.71X |
| DHX36 | 1.71X |
| DDX21 | 1.70X |
| MIS18A | 1.70X |
| LAS1L | 1.70X |
| EP400 | 1.69X |
| HUWE1 | 1.68X |
| DDX5 | 1.68X |
| ELP3 | 1.67X |
| SART3 | 1.67X |
| CPSF3 | 1.67X |
| SEM1 | 1.66X |
| BPTF | 1.66X |
| CENPW | 1.66X |
| NAT10 | 1.65X |
| CEP63 | 1.65X |
| PPP1R8 | 1.64X |
| CCNA2 | 1.63X |
| GRWD1 | 1.63X |
| DDX27 | 1.63X |
| TFPT | 1.62X |
| REXO2 | 1.61X |
| MYBBP1A | 1.61X |
| TAF9 | 1.60X |
| HUS1 | 1.60X |
| DDX54 | 1.60X |
| SMC6 | 1.60X |
| MCM2 | 1.59X |
| PELO | 1.59X |
| SMC2 | 1.59X |
| NCAPH | 1.59X |
| APAF1 | 1.58X |
| RAD9A | 1.58X |
| POLR2B | 1.57X |
| NSMCE4A | 1.57X |
| RBMX | 1.57X |
| HLTF | 1.57X |
| WAPL | 1.56X |
| DDX49 | 1.55X |
| TSEN2 | 1.56X |
| GTF2H3 | 1.55X |
| DFFA | 1.55X |
| POLR2A | 1.54X |
| MAU2 | 1.54X |
| BRCA2 | 1.53X |
| POP7 | 1.53X |
| AATF | 1.53X |
| NSMCE2 | 1.53X |
| ASF1A | 1.53X |
| GTF3C4 | 1.53X |
| TIMELESS | 1.52X |
| URI1 | 1.52X |
| NONO | 1.52X |
| DSCC1 | 1.51X |
| DDX10 | 1.51X |
| PRKDC | 1.51X |
| SSRP1 | 1.49X |
| SMC5 | 1.49X |
| SGO1 | 1.49X |
| CHTF18 | 1.49X |
| MCM3 | 1.48X |
| ISG20L2 | 1.48X |
| PRMT1 | 1.48X |
| MCM4 | 1.47X |
| PWP1 | 1.47X |
| PAXBP1 | 1.46X |
| FIP1L1 | 1.46X |
| UBE2S | 1.46X |
| NCAPG | 1.46X |
| OGA | 1.46X |
| POLN | 1.45X |
| SMC1A | 1.45X |
| POLR2K | 1.45X |
| BRD8 | 1.45X |
| ASCC3 | 1.45X |
| POLR2D | 1.44X |
| SUPT6H | 1.44X |
| DDX18 | 1.44X |
| SMC4 | 1.44X |
| RAD1 | 1.44X |
| RBBP8 | 1.44X |
| DDX51 | 1.43X |
| DDX24 | 1.43X |
| ACTR6 | 1.43X |
| POLR2L | 1.43X |
| MCM6 | 1.43X |
| HIRA | 1.43X |
| RNF113A | 1.42X |
| RAD51 | 1.43X |
| POLR2E | 1.41X |
| PALB2 | 1.40X |
| POLR2C | 1.40X |
| SUPT5H | 1.40X |
| POLR2G | 1.39X |
| SUPT16H | 1.39X |
| RUVBL1 | 1.39X |
| MMS22L | 1.39X |
| DDX56 | 1.39X |
| XPC | 1.38X |
| DHX33 | 1.38X |
| DHX37 | 1.38X |
| BAP1 | 1.38X |
| BRD9 | 1.38X |
| MCM5 | 1.38X |
| COPS6 | 1.37X |
| NCAPD2 | 1.37X |
| ABCE1 | 1.37X |
| MRGBP | 1.37X |
| PSME3 | 1.37X |
| RNF8 | 1.37X |
| BOD1L1 | 1.37X |
| CTCF | 1.36X |
| EXOSC3 | 1.36X |
| NOB1 | 1.35X |
| PCID2 | 1.35X |
| TTI2 | 1.35X |
| TRIP12 | 1.35X |
| MCM10 | 1.35X |
| DHX29 | 1.35X |
| SAP18 | 1.35X |
| SND1 | 1.34X |
| EXOSC10 | 1.34X |
| CASP3 | 1.34X |
| CLSPN | 1.34X |
| BUB1B | 1.33X |
| UHRF1 | 1.33X |
| DUT | 1.33X |
| ACTL6A | 1.33X |
| MUS81 | 1.32X |
| BRD2 | 1.32X |
| BRDT | 1.32X |
| KDM4D | 1.32X |
| MYC | 1.31X |
| MSH3 | 1.31X |
| ALYREF | 1.31X |
| DDX1 | 1.31X |
| PAF1 | 1.31X |
| HCFC1 | 1.31X |
| SLF2 | 1.31X |
| XAB2 | 1.31X |
| THRAP3 | 1.30X |
| CDC5L | 1.30X |
| NAE1 | 1.30X |
| DDX46 | 1.30X |
| E2F1 | 1.30X |
| SMARCE1 | 1.30X |
| RMI2 | 1.29X |
| ATM | 1.29X |
| DDX31 | 1.29X |
| RBBP4 | 1.29X |
| UBA3 | 1.29X |
| HDAC3 | 1.29X |
| CHTF8 | 1.28X |
| KDM5C | 1.28X |
| TCEA1 | 1.28X |
| SMARCD1 | 1.28X |
| COPS8 | 1.28X |
| COPS5 | 1.28X |
| PRPF19 | 1.28X |
| DYNLL1 | 1.27X |
| GTF2H5 | 1.27X |
| CTNNB1 | 1.27X |
| EXOSC9 | 1.27X |
| YEATS4 | 1.27X |
| PSMD14 | 1.27X |
| NASP | 1.27X |
| FANCE | 1.27X |
| CABIN1 | 1.27X |
| ACTR8 | 1.26X |
| SLX4 | 1.26X |
| SUPT4H1 | 1.26X |
| USP37 | 1.26X |
| POLR2F | 1.26X |
| COPS4 | 1.26X |
| RPA1 | 1.26X |
| REC8 | 1.26X |
| NOTCH3 | 1.26X |
| EID3 | 1.26X |
| RTF1 | 1.25X |
| CNOT4 | 1.25X |
| DDX47 | 1.25X |
| PPARG | 1.25X |
| DNMT1 | 1.25X |
| DMAP1 | 1.25X |
| SUZ12 | 1.25X |
| SOD1 | 1.25X |
| SNRNP200 | 1.25X |
| EXOSC7 | 1.25X |
| SMARCC1 | 1.25X |
| GPI | 1.25X |
| SMARCAL1 | 1.24X |
| TP53BP1 | 1.24X |
| ORC2 | 1.24X |
| PWWP3A | 1.24X |
| SENP6 | 1.24X |
| RMI1 | 1.24X |
| TERF2IP | 1.24X |
| TCOF1 | 1.24X |
| EXO1 | 1.24X |
| EWSR1 | 1.24X |
| ZZZ3 | 1.23X |
| RPS3 | 1.23X |
| RFWD3 | 1.23X |
| KEAP1 | 1.23X |
| TOPBP1 | 1.23X |
| DHX38 | 1.23X |
| RB1 | 1.23X |
| H3-3A | 1.23X |
| TTF2 | 1.22X |
| CENPC | 1.22X |
| SHLD1 | 1.22X |
| DHX9 | 1.22X |
| H2AX | 1.22X |
| ERCC4 | 1.22X |
| SCAF8 | 1.22X |
| YEATS2 | 1.22X |
| FANCC | 1.21X |
| CDC7 | 1.21X |
| SAP130 | 1.21X |
| DHX15 | 1.21X |
| POLG2 | 1.21X |
| RFC5 | 1.21X |
| UBE2W | 1.21X |
| SF3B1 | 1.21X |
| ERI1 | 1.21X |
| MSH4 | 1.20X |
| C17orf49 | 1.20X |
| ERCC6L | 1.20X |
| CUL2 | 1.20X |
| DDB1 | 1.20X |
| TOP1 | 1.20X |
| MVP | 1.20X |

TABLE X4
Genes with decreased editing efficiency
upon TwinPE CRISPRi knockdown

| | TwinPE (Approximate fold-change, |
|---|---|
| Genes | Decreased editing upon CRISPRi KD) |
| HELQ | 0.48X |
| TAF6L | 0.54X |
| CCNC | 0.66X |
| DPY30 | 0.70X |
| PARP1 | 0.73X |
| ESPL1 | 0.73X |
| PNPT1 | 0.77X |
| KDM1A | 0.77X |
| PABIR1 | 0.78X |
| METTL3 | 0.79X |
| SET | 0.80X |
| KAT7 | 0.80X |
| CDK8 | 0.81X |
| KMT5B | 0.81X |
| RFC1 | 0.82X |
| SUPT20H | 0.82X |
| RNASEH1 | 0.82X |
| POLA1 | 0.82X |
| XRCC1 | 0.82X |
| DDX3X | 0.83X |
| HMGB2 | 0.83X |
| RRM1 | 0.83X |
| HELQ | 0.48X |
| TAF6L | 0.54X |
| CCNC | 0.66X |
| DPY30 | 0.70X |
| PARP1 | 0.73X |
| ESPL1 | 0.73X |
| PNPT1 | 0.77X |
| PABIR1 | 0.78X |
| METTL3 | 0.79X |
| SET | 0.80X |
| KAT7 | 0.80X |
| CDK8 | 0.81X |
| KMT5B | 0.81X |
| RFC1 | 0.82X |
| SUPT20H | 0.82X |
| RNASEH1 | 0.82X |
| POLA1 | 0.82X |
| XRCC1 | 0.82X |
| DDX3X | 0.83X |
| HMGB2 | 0.83X |
| RRM1 | 0.83X |
| DXO | 0.94X |

TABLE X5
Genes with increased editing efficiency
upon PrimeDEL CRISPRi knockdown

| | PrimeDEL (Approximate fold-change, Increased |
|---|---|
| Genes | editing upon CRISPRi KD) |
| XRN2 | 2.85X |
| CHAF1B | 2.33X |
| UBE2T | 1.99X |
| DBR1 | 1.99X |
| RAD17 | 1.94X |
| FANCI | 1.85X |
| FANCD2 | 1.80X |
| RAD9A | 1.77X |
| CDK12 | 1.76X |
| FANCM | 1.76X |
| CHAF1A | 1.75X |
| SLX4 | 1.75X |
| FANCE | 1.73X |
| TIMELESS | 1.70X |
| FANCF | 1.69X |
| FANCB | 1.67X |
| NSMCE1 | 1.66X |
| FAAP24 | 1.66X |
| RFWD3 | 1.65X |
| SMC6 | 1.64X |
| SLBP | 1.63X |
| FANCA | 1.63X |
| FANCG | 1.62X |
| GTF2H4 | 1.62X |
| RMI1 | 1.62X |
| MCM2 | 1.62X |
| HUS1 | 1.62X |
| SMC5 | 1.62X |
| SMARCA5 | 1.60X |
| DDX20 | 1.60X |
| FANCC | 1.59X |
| HINFP | 1.58X |
| DUT | 1.58X |
| GTF2F2 | 1.55X |
| NSMCE4A | 1.55X |
| PPP1R8 | 1.54X |
| FANCL | 1.53X |
| NOP10 | 1.52X |
| RAD51 | 1.52X |
| HLTF | 1.52X |
| RBBP4 | 1.51X |
| INTS3 | 1.51X |
| ERCC4 | 1.51X |
| TAF9 | 1.51X |
| CCNH | 1.50X |
| NONO | 1.50X |
| MCM4 | 1.50X |
| MCM6 | 1.49X |
| USP37 | 1.49X |
| MCM10 | 1.49X |
| RPP21 | 1.49X |
| CPSF3 | 1.48X |
| TAF1 | 1.48X |
| SENP6 | 1.47X |
| RPP40 | 1.46X |
| FAAP100 | 1.46X |
| POP5 | 1.45X |
| SLF2 | 1.45X |
| CENPA | 1.45X |
| RPP30 | 1.44X |
| RPAIN | 1.44X |
| MNAT1 | 1.44X |
| DKC1 | 1.43X |
| HJURP | 1.43X |
| FIP1L1 | 1.42X |
| HIRA | 1.42X |
| DDX46 | 1.42X |
| DSCC1 | 1.42X |
| MCM3 | 1.42X |
| CHTF18 | 1.42X |
| DAXX | 1.41X |
| NSMCE2 | 1.41X |
| MYB | 1.41X |
| RAD1 | 1.41X |
| TRIP12 | 1.40X |
| MMS22L | 1.40X |
| HNRNPK | 1.40X |
| GTF3C4 | 1.40X |
| CFDP1 | 1.39X |
| POLR2H | 1.39X |
| DNMT1 | 1.38X |
| GAR1 | 1.38X |
| RBMX | 1.38X |
| POLD3 | 1.38X |
| RPA3 | 1.38X |
| ERCC2 | 1.38X |
| XRCC5 | 1.37X |
| PAXBP1 | 1.37X |
| SART3 | 1.37X |
| CCNA2 | 1.37X |
| PRMT1 | 1.37X |
| DHX35 | 1.36X |
| ACTR6 | 1.36X |
| WDHD1 | 1.36X |
| RBBP8 | 1.36X |
| SEM1 | 1.35X |
| POLE | 1.35X |
| DHX36 | 1.35X |
| MIS18A | 1.35X |
| TREX2 | 1.35X |
| TFPT | 1.35X |
| NHP2 | 1.35X |
| CCAR1 | 1.35X |
| ATRX | 1.35X |
| RCL1 | 1.34X |
| EXOSC2 | 1.34X |
| CTC1 | 1.34X |
| SRSF1 | 1.34X |
| ASF1A | 1.34X |
| ATRIP | 1.34X |
| RPP14 | 1.34X |
| POLE2 | 1.34X |
| CLSPN | 1.34X |
| BRCA2 | 1.33X |
| BOD1L1 | 1.33X |
| POLA2 | 1.33X |
| POP4 | 1.33X |
| MCM5 | 1.33X |
| CABIN1 | 1.33X |
| GPN1 | 1.33X |
| POT1 | 1.33X |
| XRCC6 | 1.32X |
| TYMS | 1.32X |
| WAPL | 1.32X |
| TOPBP1 | 1.32X |
| UHRF1 | 1.32X |
| CUL2 | 1.32X |
| NCAPH | 1.31X |
| VPS72 | 1.31X |
| ERCC3 | 1.31X |
| BPTF | 1.30X |
| DROSHA | 1.30X |
| RPP38 | 1.30X |
| SETDB1 | 1.30X |
| EXOSC5 | 1.30X |
| ACD | 1.30X |
| DICER1 | 1.30X |
| EXOSC4 | 1.30X |
| AATF | 1.30X |
| DDX41 | 1.30X |
| CNOT4 | 1.29X |
| LAS1L | 1.29X |
| CEP63 | 1.29X |
| DDX23 | 1.29X |
| DDX5 | 1.29X |
| NCAPD2 | 1.29X |
| RMI2 | 1.29X |
| CENPW | 1.29X |
| MSH3 | 1.28X |
| HNRNPC | 1.28X |
| EXOSC9 | 1.28X |
| POP1 | 1.28X |
| RAD51C | 1.28X |
| PCID2 | 1.27X |
| POLR2A | 1.27X |
| PALB2 | 1.27X |
| HDAC3 | 1.27X |
| ZNHIT1 | 1.27X |
| RFC5 | 1.26X |
| ERCC6L | 1.26X |
| SSRP1 | 1.26X |
| COPS6 | 1.26X |
| POP7 | 1.25X |
| OASL | 1.25X |
| POLR2B | 1.25X |
| DYNLL1 | 1.25X |
| POLN | 1.25X |
| DEK | 1.25X |
| DFFA | 1.25X |
| SFPQ | 1.25X |
| GTF2H3 | 1.25X |
| YEATS4 | 1.24X |
| DDX49 | 1.24X |
| DIS3 | 1.24X |
| ATR | 1.24X |
| BRIP1 | 1.24X |
| ATM | 1.24X |
| URI1 | 1.24X |
| CENPP | 1.24X |
| DHX37 | 1.23X |
| ORC2 | 1.23X |
| PWWP3A | 1.23X |
| NAE1 | 1.23X |
| RFC3 | 1.23X |
| RFC2 | 1.23X |
| DDX10 | 1.23X |
| POLR2C | 1.23X |
| CDC5L | 1.23X |
| DDX39B | 1.22X |
| CHD4 | 1.22X |
| POLR2I | 1.22X |
| KMT2B | 1.22X |
| THRAP3 | 1.22X |
| GTF2H1 | 1.22X |
| CDK7 | 1.22X |
| TCEA1 | 1.22X |
| SCAF8 | 1.22X |
| PIAS1 | 1.21X |
| ZBTB7A | 1.21X |
| DHX15 | 1.21X |
| OGA | 1.21X |
| CDC7 | 1.21X |
| TIPIN | 1.21X |
| EXOSC3 | 1.21X |
| RNF113A | 1.21X |
| SAE1 | 1.21X |
| MYBBP1A | 1.21X |
| NAT10 | 1.21X |
| DDX51 | 1.21X |
| PELO | 1.21X |
| PAF1 | 1.20X |
| SETD1A | 1.20X |
| ELAC2 | 1.20X |
| RPA1 | 1.20X |
| HUWE1 | 1.20X |
| UBA3 | 1.20X |
| DDX21 | 1.20X |

TABLE X6
Genes with decreased editing efficiency
upon PrimeDEL CRISPRi knockdown

| | PrimeDEL (Approximate fold-change, |
|---|---|
| Genes | Decreased editing upon CRISPRi KD) |
| TAF6L | 0.54X |
| KDM1A | 0.66X |
| ESPL1 | 0.75X |
| PABIR1 | 0.78X |
| SUPT20H | 0.80X |
| CNOT1 | 0.80X |
| DR1 | 0.81X |
| SSBP3 | 0.81X |
| RNASEH1 | 0.82X |
| BTAF1 | 0.82X |
| HELQ | 0.92X |
| DXO | 0.92X |

TABLE X7
Genes with increased editing efficiency
upon PE3 CRISPRi knockdown

| | PE3 (Approximate fold-change, Increased |
|---|---|
| Genes | editing upon CRISPRi KD) |
| DBR1 | 2.99X |
| PMS2 | 2.53X |
| XRN2 | 2.52X |
| EXOSC2 | 2.46X |
| MSH6 | 2.43X |
| MLH1 | 2.37X |
| EXOSC5 | 2.36X |
| MSH2 | 2.32X |
| EXOSC4 | 2.21X |
| NSMCE1 | 2.00X |
| SMC6 | 2.00X |
| NOP10 | 1.99X |
| DIS3 | 1.98X |
| SMC5 | 1.95X |
| EXOSC3 | 1.93X |
| DKC1 | 1.84X |
| XRCC6 | 1.81X |
| SMARCA5 | 1.81X |
| NSMCE4A | 1.79X |
| TAF1 | 1.78X |
| EXO1 | 1.76X |
| GTF3C4 | 1.75X |
| RCL1 | 1.74X |
| SMC1A | 1.74X |
| GAR1 | 1.73X |
| XRCC5 | 1.73X |
| NSMCE2 | 1.71X |
| SLF2 | 1.63X |
| CHAF1B | 1.63X |
| DDX24 | 1.56X |
| DDX27 | 1.56X |
| CENPP | 1.55X |
| BPTF | 1.54X |
| EXOSC10 | 1.53X |
| AATF | 1.53X |
| NONO | 1.52X |
| DHX37 | 1.52X |
| NHP2 | 1.52X |
| NELFB | 1.52X |
| DDX21 | 1.51X |
| NAT10 | 1.50X |
| MYBBP1A | 1.50X |
| EXOSC9 | 1.50X |
| DDX51 | 1.50X |
| PPP1R8 | 1.50X |
| MTREX | 1.49X |
| EXOSC7 | 1.48X |
| HIRA | 1.48X |
| MYB | 1.46X |
| CABIN1 | 1.43X |
| FIP1L1 | 1.43X |
| LAS1L | 1.43X |
| CENPA | 1.43X |
| DDX18 | 1.42X |
| HNRNPC | 1.42X |
| ATRX | 1.41X |
| HDAC3 | 1.41X |
| MCM2 | 1.41X |
| CDK12 | 1.41X |
| ASF1A | 1.41X |
| RAD51 | 1.41X |
| DDX49 | 1.40X |
| RNF4 | 1.40X |
| DHX35 | 1.40X |
| SAP18 | 1.40X |
| GTF2F2 | 1.40X |
| DDX10 | 1.39X |
| DUT | 1.39X |
| DDX56 | 1.39X |
| PNPT1 | 1.39X |
| NCAPD2 | 1.37X |
| MCM6 | 1.37X |
| CNOT4 | 1.37X |
| HJURP | 1.36X |
| SAE1 | 1.36X |
| TFPT | 1.36X |
| DHX36 | 1.36X |
| DAXX | 1.35X |
| WAPL | 1.35X |
| PWWP3A | 1.35X |
| DDX47 | 1.34X |
| POLD3 | 1.34X |
| CCNH | 1.34X |
| DPF1 | 1.34X |
| UHRF1 | 1.34X |
| BRCA2 | 1.33X |
| DHX30 | 1.33X |
| MSH3 | 1.32X |
| SCAF8 | 1.32X |
| CFDP1 | 1.32X |
| DDX20 | 1.32X |
| POLR2A | 1.32X |
| TAF9 | 1.32X |
| SLBP | 1.32X |
| VPS72 | 1.31X |
| DDX5 | 1.31X |
| DHX29 | 1.31X |
| MCM4 | 1.31X |
| DHX16 | 1.31X |
| CEP63 | 1.30X |
| DHX8 | 1.30X |
| DNMT1 | 1.30X |
| SSRP1 | 1.30X |
| NOB1 | 1.30X |
| STAG3 | 1.30X |
| GTF2H4 | 1.30X |
| ACTR8 | 1.30X |
| PRMT1 | 1.30X |
| POLR2H | 1.29X |
| DDX54 | 1.29X |
| CCNA2 | 1.29X |
| ACTR6 | 1.29X |
| BCCIP | 1.28X |
| DYNLL1 | 1.28X |
| DHX33 | 1.28X |
| TIMELESS | 1.28X |
| RBMX | 1.27X |
| CUL2 | 1.27X |
| NOTCH3 | 1.27X |
| PIAS1 | 1.27X |
| EP400 | 1.26X |
| POLR2G | 1.26X |
| DDX41 | 1.26X |
| SART3 | 1.26X |
| TREX2 | 1.26X |
| DEK | 1.26X |
| KDM5C | 1.25X |
| KMT2B | 1.25X |
| HINFP | 1.25X |
| TCEA1 | 1.25X |
| DDX39B | 1.25X |
| XPC | 1.25X |
| MCM10 | 1.25X |
| SND1 | 1.25X |
| ISG20L2 | 1.23X |
| MCM3AP | 1.23X |
| INTS3 | 1.23X |
| MNAT1 | 1.23X |
| RBBP8 | 1.23X |
| BCAS2 | 1.23X |
| TYMS | 1.22X |
| ELP3 | 1.22X |
| PELO | 1.22X |
| LMNA | 1.22X |
| COPS6 | 1.22X |
| SFPQ | 1.22X |
| DHX40 | 1.22X |
| CTCF | 1.22X |
| SOD1 | 1.22X |
| CHAF1A | 1.21X |
| SEM1 | 1.21X |
| UBE2I | 1.21X |
| PAXBP1 | 1.21X |
| SSBP1 | 1.21X |
| DDX31 | 1.21X |
| TRIP12 | 1.21X |
| IGHMBP2 | 1.21X |
| BRD9 | 1.20X |
| GRWD1 | 1.20X |
| MIS18A | 1.20X |

TABLE X8
Genes with decreased editing efficiency
upon PE3 CRISPRi knockdown

| | PrimeDEL (Approximate fold-chaange, |
|---|---|
| Genes | Decreased editing upon CRISPRi KD) |
| TAF6L | 0.46X |
| KDM1A | 0.54X |
| BTAF1 | 0.56X |
| FEN1 | 0.61X |
| DR1 | 0.72X |
| RPP21 | 0.73X |
| LIG1 | 0.74X |
| ESPL1 | 0.75X |
| SSBP3 | 0.77X |
| SLF1 | 0.79X |
| PABIR1 | 0.79X |
| MEAF6 | 0.79X |
| CXXC1 | 0.80X |
| PBRM1 | 0.80X |
| LIG3 | 0.83X |
| DOX | 0.84X |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim may be modified to include one or more limitations found in any other claim that is dependent on the same base claim. Where elements are presented as lists, e.g., in Markush group format, each subgroup of the elements is also disclosed, and any element(s) may be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention may be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A system for simultaneously editing both strands of a double-stranded DNA sequence at a target site to be edited, the system comprising:
(a) a first prime editor comprising:
(i) a first prime editor comprising a first nucleic acid programmable DNA binding protein (first napDNAbp) and a first polypeptide comprising an RNA-dependent DNA polymerase activity, or one or more polynucleotides encoding the first prime editor; and
(ii) a first prime editing guide RNA (first pegRNA) that binds to a first binding site on a first strand of the double-stranded DNA sequence upstream of the target site to be edited, or one or more polynucleotides encoding the first pegRNA;
(b) a second prime editor comprising:
(i) a second prime editor comprising a second nucleic acid programmable DNA binding protein (second napDNAbp) and a second polypeptide comprising an RNA-dependent DNA polymerase activity, or one or more polynucleotides encoding the second prime editor; and
(ii) a second prime editing guide RNA (second pegRNA) that binds to a second binding site on a second strand of the double-stranded DNA sequence upstream of the target site to be edited, or one or more polynucleotides encoding the second pegRNA; and
(c) one or more inhibitors of one or more genes or gene products, wherein inhibiting the one or more genes or gene products increases the editing efficiency of the system by at least 1.3 fold;
wherein the first pegRNA comprises a first DNA synthesis template encoding a first single-stranded DNA sequence, and the second pegRNA comprises a second DNA synthesis template encoding a second single-stranded DNA sequence, and wherein the first and the second single-stranded DNA sequence each comprise a region of complementarity to the other, and wherein the first single-stranded DNA sequence and the second single-stranded DNA sequence form a duplex comprising an edited portion as compared to the DNA sequence at the target site to be edited.

2. A system for simultaneously editing both strands of a double-stranded DNA sequence at a target site to be edited, the system comprising:
(a) a first prime editor comprising:
(i) a first prime editor comprising a first nucleic acid programmable DNA binding protein (first napDNAbp) and a first polypeptide comprising an RNA-dependent DNA polymerase activity, or one or more polynucleotides encoding the first prime editor; and
(ii) a first prime editing guide RNA (first pegRNA) comprising a first spacer sequence, a first gRNA core, a first DNA synthesis template, and a first primer binding site that binds to a first binding site on a first strand of the double-stranded DNA sequence upstream of the target site to be edited, or one or more polynucleotides encoding the first pegRNA
(b) a second prime editor comprising:
(i) a second prime editor comprising a second nucleic acid programmable DNA binding protein (second napDNAbp) and a second polypeptide comprising an RNA-dependent DNA polymerase activity, or one or more polynucleotides encoding the second prime editor; and
(ii) a second prime editing guide RNA (second pegRNA) comprising a second spacer sequence, a second gRNA core, a second DNA synthesis template, and a second primer binding site that binds to a second binding site on a second strand of the double-stranded DNA sequence upstream of the target site to be edited, or one or more polynucleotides encoding the second pegRNA; and
(c) one or more inhibitors of one or more genes or gene products, wherein inhibiting the one or more genes or gene products increases the editing efficiency of the system by at least 1.3 fold;
wherein the first DNA synthesis template encodes a first single-stranded DNA and the second DNA synthesis template encodes a second single-stranded DNA,
wherein the first single-stranded DNA sequence and the second single-stranded DNA sequence each comprise a region of complementarity to the other,
wherein the first single-stranded DNA sequence does not have a region of complementarity to the second primer,
wherein the second single-stranded DNA sequence does not have a region of complementarity to the first primer, and
wherein the first single-stranded DNA sequence and the second single-stranded DNA sequence form a duplex comprising an edited portion as compared to the DNA sequence at the target site to be edited.

3. The systems of claim 1 or 2, wherein the one or more genes or gene products are listed in Table 1.

4. A system for simultaneously editing both strands of a double-stranded DNA sequence at a target site to be edited, the system comprising:
(a) a first prime editor comprising:
(i) a first prime editor comprising a first nucleic acid programmable DNA binding protein (first napDNAbp) and a first polypeptide comprising an RNA-dependent DNA polymerase activity, or one or more polynucleotides encoding the first prime editor; and
(ii) a first prime editing guide RNA (first pegRNA) that binds to a first binding site on a first strand of the double-stranded DNA sequence upstream of the target site to be edited, or one or more polynucleotides encoding the first pegRNA;
(b) a second prime editor comprising:
(i) a second prime editor comprising a second nucleic acid programmable DNA binding protein (second napDNAbp) and a second polypeptide comprising an RNA-dependent DNA polymerase activity or one or more polynucleotides encoding the second prime editor; and
(ii) a second prime editing guide RNA (second pegRNA) that binds to a second binding site on a second strand of the double-stranded DNA sequence upstream of the target site to be edited, or one or more polynucleotides encoding the second pegRNA; and
(c) one or more inhibitors of one or more genes or gene products, wherein inhibiting the one or more genes or gene products increases the editing efficiency of the system by at least 1.3 fold;
wherein the first pegRNA comprises a first DNA synthesis template comprises a region of complementary to the second binding site of the second pegRNA and the second pegRNA comprises a second DNA synthesis template comprises a region of complementary to the first binding site of the first pegRNA.

5. A system for simultaneously editing both strands of a double-stranded DNA sequence at a target site to be edited, the system comprising:
(a) a first prime editor comprising:
(i) a first prime editor comprising a first nucleic acid programmable DNA binding protein (first napDNAbp) and a first polypeptide comprising an RNA-dependent DNA polymerase activity, or one or more polynucleotides encoding the first prime editor; and
(ii) a first prime editing guide RNA (first pegRNA) comprising a first spacer sequence, a first gRNA core, a first DNA synthesis template, and a first primer binding site, wherein the first primer binding site binds to a first primer on a first strand of the double-stranded DNA sequence upstream of the target site to be edited, or one or more polynucleotides encoding the first pegRNA;
(b) a second prime editor comprising:
(i) a second prime editor comprising a second nucleic acid programmable DNA binding protein (second napDNAbp) and a second polypeptide comprising an RNA-dependent DNA polymerase activity, or one or more polynucleotides encoding the second prime editor; and
(ii) a second prime editing guide RNA (second pegRNA) comprising a second spacer sequence, a second gRNA core, a second DNA synthesis template, and a second primer binding site, wherein the second primer binding site binds to a second primer on a second strand of the double-stranded DNA sequence upstream of the target site to be edited, or one or more polynucleotides encoding the second pegRNA; and
(c) one or more inhibitors of one or more genes or gene products, wherein inhibiting the one or more genes or gene products increases the editing efficiency of the system by at least 1.3 fold;
wherein the first DNA synthesis template encodes a first single-stranded DNA, and the second DNA synthesis template encodes a second single-stranded DNA,
wherein the first single-stranded DNA sequence and the second single-stranded DNA sequence each comprise a region of complementarity to the other,
wherein the first single-stranded DNA sequence comprises a region of complementarity to the second primer,
wherein the second single-stranded DNA sequence comprises a region of complementarity to the first primer, and
wherein the first single-stranded DNA sequence and the second single-stranded DNA sequence form a duplex comprising an edited portion as compared to the DNA sequence at the target site to be edited.

6. The system of claim 4 or 5 wherein the one or more genes or gene products are listed in Table 2.

7. A system for editing a nucleic acid molecule by prime editing comprising a prime editor, a pegRNA, a sgRNA, and one or more inhibitors of one or more genes or gene products, wherein inhibiting the one or more genes or gene products increases the editing efficiency of the system by at least 1.3 fold, wherein the system is capable of installing one or more modification to the nucleic acid molecule at a target site.

8. The system of claim 7, wherein the one or more genes or gene products are listed in Table 1.

9. A composition comprising a system of any one of claim 1-2 or 4-5 and one or more inhibitors of one or more genes or gene products listed in Tables 4, X1 and X2, wherein the composition is capable of installing one or more modifications to a nucleic acid molecule at a target site.

10. A composition comprising a system of any one of claim 1-2 or 7, and one or more inhibitors of one or more genes or gene products listed in Tables 5, X1 and X2, wherein the composition is capable of installing one or more modifications to a nucleic acid molecule at a target site.

11. A composition comprising a system of any one of claim 4-5 or 7 and one or more inhibitors of one or more genes or gene products listed in Tables 6, X1 and X2, wherein the composition is capable of installing one or more modifications to a nucleic acid molecule at a target site.

12. A composition comprising a system of any one of claims 1-2, 4-5, or claim 7 and one or more inhibitors of one or more genes or gene products listed in Tables 7, X1 and X2, wherein the composition is capable of installing one or more modifications to a nucleic acid molecule at a target site.

13. A composition comprising a system of any one of claim 1 or 2 and an inhibitor of one or more complexes comprising one or more gene products from one or more genes listed in Table 1.

14. A composition comprising a system of any one of claims 4-5 and an inhibitor of one or more complexes comprising one or more gene products from one or more genes listed in Table 2.

15. A composition comprising a system of claim 7 and an inhibitor of one or more complexes comprising one or more gene products from one or more genes listed in Table 3.

16. A composition comprising a system of any one of claim 1 or 2 and an inhibitor of one or more pathways comprising one or more gene products from one or more genes listed in Table 1.

17. A composition comprising a system of any one of claims 4-5 and an inhibitor of one or more pathways comprising one or more gene products from one or more genes listed in Table 2.

18. A composition comprising a system of claim 7 and an inhibitor of one or more pathways comprising one or more gene products from one or more genes listed in Table 3.

19. The system or composition of any one of claims 1-18, wherein the inhibitor is an inhibitor identified using CRISPRi.

20. The system or composition of any one of claims 1-19, wherein the inhibitor comprises an RNA interference (RNAi).

21. The system or composition of claim 20, wherein the RNAi is a siRNA

22. The system or composition of claim 20 or 21, wherein the RNAi is a microRNA (miRNA).

23. The system or composition of any one of claims 1-22, wherein the inhibitor comprises a small molecule inhibitor.

24. The system or composition of claim 23, wherein the small molecule inhibitor is a covalent inhibitor.

25. The system or composition of any one of claims 1-24, wherein the small molecule inhibitor is a non-covalent inhibitor.

26. The system or composition of any one of claims 1-25, wherein the inhibitor comprises an antibody or a fragment thereof.

27. The system or composition of any one of claims 1-26, wherein the inhibitor comprises a dominant negative gene product of one of the genes listed in Tables 1-7.

28. A polynucleotide encoding a system or composition of any one of claims 1-27.

29. A polynucleotide encoding a system of any one of claim 1-2 or 19-27.

30. A polynucleotide encoding a system of any one of claim 4-5 or 19-27.

31. A polynucleotide encoding a system of claim 5- or 19-27.

32. A polynucleotide encoding a composition of any one of claims 9 and 19-27.

33. A polynucleotide encoding a composition of any one of claims 10 and 19-27.

34. A polynucleotide encoding a composition of any one of claims 11 and 19-27.

35. A polynucleotide encoding a composition of any one of claims 12 and 19-27.

36. A polynucleotide encoding a composition of any one of claims 13-27.

37. A vector comprising a polynucleotide of any one of claims 28-36.

38. A cell comprising a polynucleotide of any one of claims 28-36 or a vector of 37.

39. A pharmaceutical composition comprising a polynucleotide of any one of claims 26-36, a vector of 37, or a cell of claim 38, and a pharmaceutical excipient.

40. A kit comprising a polynucleotide of any of 28-36 or a vector of claim 37 and instructions for simultaneously editing both strands of a double-stranded DNA sequence at a target site to be edited.

41. A method for editing a nucleic acid molecule by prime editing, the method comprising contacting a nucleic acid with a system or composition of any one of claims 1-27, a polynucleotide of any one of claims 28-36, a vector of claim 37, or a pharmaceutical composition of claim 39.

42. The method of claim 38, wherein the nucleic acid is a double stranded DNA.

43. A composition for simultaneously editing both strands of a double-stranded DNA sequence at a target site to be edited, the composition comprising:
(a) a first prime editor comprising:
(i) a first prime editor comprising a first nucleic acid programmable DNA binding protein (first napDNAbp) and a first polypeptide comprising an RNA-dependent DNA polymerase activity, or one or more polynucleotides encoding the first prime editor; and
(ii) a first prime editing guide RNA (first pegRNA) that binds to a first binding site on a first strand of the double-stranded DNA sequence upstream of the target site to be edited, or one or more polynucleotides encoding the first pegRNA;
(b) a second prime editor comprising:
(i) a second prime editor comprising a second nucleic acid programmable DNA binding protein (second napDNAbp) and a second polypeptide comprising an RNA-dependent DNA polymerase activity, or one or more polynucleotides encoding the second prime editor; and
(ii) a second prime editing guide RNA (second pegRNA) that binds to a second binding site on a second strand of the double-stranded DNA sequence upstream of the target site to be edited, or one or more polynucleotides encoding the second pegRNA; and
(c) one or more enhancers of one or more genes or gene products, wherein the one or more gene products is any one of the gene products listed in Table X4, wherein the first pegRNA comprises a first DNA synthesis template encoding a first single-stranded DNA sequence, and the second pegRNA comprises a second DNA synthesis template encoding a second single-stranded DNA sequence, and wherein the first and the second single-stranded DNA sequence each comprise a region of complementarity to the other, and wherein the first single-stranded DNA sequence and the second single-stranded DNA sequence form a duplex comprising an edited portion as compared to the DNA sequence at the target site to be edited.

44. A composition for simultaneously editing both strands of a double-stranded DNA sequence at a target site to be edited, the composition comprising:
(a) a first prime editor comprising:
(i) a first prime editor comprising a first nucleic acid programmable DNA binding protein (first napDNAbp) and a first polypeptide comprising an RNA-dependent DNA polymerase activity, or one or more polynucleotides encoding the first prime editor; and
(ii) a first prime editing guide RNA (first pegRNA) comprising a first spacer sequence, a first gRNA core, a first DNA synthesis template, and a first primer binding site, wherein the first primer binding site binds to a first primer on a first strand of the double-stranded DNA sequence upstream of the target site to be edited, or one or more polynucleotides encoding the first pegRNA;
(b) a second prime editor comprising:
(i) a second prime editor comprising a second nucleic acid programmable DNA binding protein (second napDNAbp) and a second polypeptide comprising an RNA-dependent DNA polymerase activity, or one or more polynucleotides encoding the second prime editor; and
(ii) a second prime editing guide RNA (second pegRNA) comprising a second spacer sequence, a second gRNA core, a second DNA synthesis template, and a second primer binding site, wherein the second primer binding site binds to a second primer on a second strand of the double-stranded DNA sequence upstream of the target site to be edited, or one or more polynucleotides encoding the second pegRNA; and
(c) one or more enhancers of one or more genes or gene products, wherein the one or more gene products is any one of the gene products listed in Table X4,
wherein the first DNA synthesis template encodes a first single-stranded DNA and the second DNA synthesis template encodes a second single-stranded DNA,
wherein the first single-stranded DNA sequence and the second single-stranded DNA sequence each comprise a region of complementarity to the other,
wherein the first single-stranded DNA sequence does not have a region of complementarity to the second primer,
wherein the second single-stranded DNA sequence does not have a region of complementarity to the first primer, and
wherein the first single-stranded DNA sequence and the second single-stranded DNA sequence form a duplex comprising an edited portion as compared to the DNA sequence at the target site to be edited.

45. A composition for simultaneously editing both strands of a double-stranded DNA sequence at a target site to be edited, the composition comprising:
(a) a first prime editor comprising:
(i) a first prime editor comprising a first nucleic acid programmable DNA binding protein (first napDNAbp) and a first polypeptide comprising an RNA-dependent DNA polymerase activity, or one or more polynucleotides encoding the first prime editor; and
(ii) a first prime editing guide RNA (first pegRNA) that binds to a first binding site on a first strand of the double-stranded DNA sequence upstream of the target site to be edited, or one or more polynucleotides encoding the first pegRNA;
(b) a second prime editor comprising:
(i) a second prime editor comprising a second nucleic acid programmable DNA binding protein (second napDNAbp) and a second polypeptide comprising an RNA-dependent DNA polymerase activity, or one or more polynucleotides encoding the second prime editor; and
(ii) a second prime editing guide RNA (second pegRNA) that binds to a second binding site on a second strand of the double-stranded DNA sequence upstream of the target site to be edited, or one or more polynucleotides encoding the second pegRNA; and
(c) one or more enhancers of one or more genes or gene products, wherein the one or more gene products is any one of the gene products listed in Table X6,
wherein the first pegRNA comprises a first DNA synthesis template that comprises a region of complementary to the second binding site of the second pegRNA and the second pegRNA comprises a second DNA synthesis template that comprises a region of complementary to the first binding site of the first pegRNA.

46. A composition for simultaneously editing both strands of a double-stranded DNA sequence at a target site to be edited, the composition comprising:
(a) a first prime editor comprising:
(i) a first prime editor comprising a first nucleic acid programmable DNA binding protein (first napDNAbp) and a first polypeptide comprising an RNA-dependent DNA polymerase activity, or one or more polynucleotides encoding the first prime editor; and
(ii) a first prime editing guide RNA (first pegRNA) comprising a first spacer sequence, a first gRNA core, a first DNA synthesis template, and a first primer binding site, wherein the first primer binding site binds to a first primer on a first strand of the double-stranded DNA sequence upstream of the target site to be edited, or one or more polynucleotides encoding the first pegRNA;
(b) a second prime editor comprising:
(i) a second prime editor comprising a second nucleic acid programmable DNA binding protein (second napDNAbp) and a second polypeptide comprising an RNA-dependent DNA polymerase activity, or one or more polynucleotides encoding the second prime editor; and
(ii) a second prime editing guide RNA (second pegRNA) comprising a second spacer sequence, a second gRNA core, a second DNA synthesis template, and a second primer binding site, wherein the second primer binding site binds to a second primer on a second strand of the double-stranded DNA sequence upstream of the target site to be edited, or one or more polynucleotides encoding the second pegRNA; and
(c) one or more enhancers of one or more genes or gene products, wherein the one or more gene products is any one of the gene products listed in Table X6,
wherein the first DNA synthesis template encodes a first single-stranded DNA, and the second DNA synthesis template encodes a second single-stranded DNA,
wherein the first single-stranded DNA sequence and the second single-stranded DNA sequence each comprise a region of complementarity to the other,
wherein the first single-stranded DNA sequence does have a region of complementarity to the second primer,
wherein the second single-stranded DNA sequence does have a region of complementarity to the first primer, and
wherein the first single-stranded DNA sequence and the second single-stranded DNA sequence form a duplex comprising an edited portion as compared to the DNA sequence at the target site to be edited.

47. A composition for editing a nucleic acid molecule by prime editing comprising a prime editor, a pegRNAs, and optionally a sgRNA, and one or more enhancers of one or more genes or gene products listed in Table X8, wherein the system is capable of installing one or more modifications to the nucleic acid molecule at a target site.

48. The composition of claim 47, wherein the sgRNA guides the prime editor to nick an unedited strand of the nucleic acid at a position downstream of a pegRNA nick site.

49. The composition of claim 48, wherein the sgRNA nick site is between positions −1 and −300 nts from the pegRNA nick site

50. The composition of claim 48 or 49, wherein the sgRNA nick site is between −10 and −100 nts from the pegRNA nick site.

51. The composition of claim 48-49, wherein the sgRNA nick site is between −25 and −75 nts from the pegRNA nick site.

52. The composition of any one of claims 43-47, wherein the one or more genes comprises KDM1A.

53. The composition of any one of claims 43-47, wherein the one or more genes comprises DXO.

54. The composition of any one of claims 43-47, wherein the one or more genes comprises HELQ.

55. The composition of claim 50-53, wherein the HELQ comprises a C-terminal helicase domain of HELQ.

56. The composition of claim 51, wherein the C-terminal helicase domain has helicase activity.

57. A polynucleotide encoding one or more of the compositions of any one of claims 43-56.

58. A polynucleotide encoding the composition of any one of claim 43-44 or 48-56.

59. A polynucleotide encoding the composition of any one of claim 45-46 or 48-56.

60. A polynucleotide encoding the composition of claim 47 or 48-56.

61. A vector comprising the polynucleotide of any one of claims 57-60.

62. A cell comprising the polynucleotide of any one of claims 57-60 or the vector of claim 61.

63. A pharmaceutical composition comprising the polynucleotide of any one of claims 57-60, the vector of claim 61, or the cell of claim 62, and a pharmaceutical excipient.

64. A kit comprising the polynucleotide of any of 56-59 or a vector of claim 61 and instructions for simultaneously editing both strands of a double-stranded DNA sequence at a target site to be edited.

65. A method for editing a nucleic acid molecule by prime editing, the method comprising contacting a nucleic acid with the composition of any one of claims 43-56, a polynucleotide of any one of claims 56-59, a vector of claim 61 or a pharmaceutical composition of claim 39.

66. The method of claim 65 wherein the nucleic acid is a double stranded DNA.

67. A system for simultaneously editing both strands of a double-stranded DNA sequence at a target site to be edited, the system comprising:
(i) a first prime editor comprising a first nucleic acid programmable DNA binding protein (first napDNAbp) and a first polypeptide comprising an RNA-dependent DNA polymerase activity, or one or more polynucleotides encoding the first prime editor;
(ii) a first prime editing guide RNA (first pegRNA) that binds to a first binding site on a first strand of the double-stranded DNA sequence upstream of the target site to be edited, or one or more polynucleotides encoding the first pegRNA;
iii) a second prime editor comprising a second nucleic acid programmable DNA binding protein (second napDNAbp) and a second polypeptide comprising an RNA-dependent DNA polymerase activity, or one or more polynucleotides encoding the second prime editor; and
(iv) a second prime editing guide RNA (second pegRNA) that binds to a second binding site on a second strand of the double-stranded DNA sequence upstream of the target site to be edited, or one or more polynucleotides encoding the second pegRNA; and
(v) one or more inhibitors of one or more genes or gene products listed in Table 1 or Table X3;
wherein the first pegRNA comprises a first DNA synthesis template encoding a first single-stranded DNA sequence, and the second pegRNA comprises a second DNA synthesis template encoding a second single-stranded DNA sequence, and wherein the first and the second single-stranded DNA sequence each comprise a region of complementarity to the other, and wherein the first single-stranded DNA sequence and the second single-stranded DNA sequence form a duplex comprising an edited portion as compared to the DNA sequence at the target site to be edited.

68. A system for simultaneously editing both strands of a double-stranded DNA sequence at a target site to be edited, the system comprising:
(i) a first prime editor comprising a first nucleic acid programmable DNA binding protein (first napDNAbp) and a first polypeptide comprising an RNA-dependent DNA polymerase activity, or one or more polynucleotides encoding the first prime editor; and
(ii) a first prime editing guide RNA (first pegRNA) or one or more polynucleotides encoding the first pegRNA, wherein the first pegRNA comprises a first spacer sequence, a first gRNA core, a first DNA synthesis template, and a first primer binding site that binds to a first binding site on a first strand of the double-stranded DNA sequence upstream of the target site to be edited, or one or more polynucleotides encoding the first pegRNA;
(iii) a second prime editor comprising a second nucleic acid programmable DNA binding protein (second napDNAbp) and a second polypeptide comprising an RNA-dependent DNA polymerase activity, or one or more polynucleotides encoding the second prime editor; and
(iv) a second prime editing guide RNA (second pegRNA) or one or more polynucleotides encoding the second pegRNA, wherein the second pegRNA comprises a second spacer sequence, a second gRNA core, a second DNA synthesis template, and a second primer binding site that binds to a second binding site on a second strand of the double-stranded DNA sequence upstream of the target site to be edited, or one or more polynucleotides encoding the first pegRNA; and
(v) one or more inhibitors of one or more genes or gene products listed in Table 1 or Table X3,
wherein the first DNA synthesis template encodes a first single-stranded DNA and the second DNA synthesis template encodes a second single-stranded DNA,
wherein the first single-stranded DNA sequence and the second single-stranded DNA sequence each comprise a region of complementarity to the other,
wherein the first single-stranded DNA sequence does not have a region of complementarity to the second primer,
wherein the second single-stranded DNA sequence does not have a region of complementarity to the first primer, and
wherein the first single-stranded DNA sequence and the second single-stranded DNA sequence form a duplex comprising an edited portion as compared to the DNA sequence at the target site to be edited.

69. The system of claim 67 or 68, wherein the one or more genes comprises one or more genes selected from the group consisting of CHAF1B, DBR1, XRN2, MNAT1, CCNH, GTF2H1, GTF2H4, NOP10, RPP21, POP5, GRF2F2, TTI1, DDX20, and DROSHA.

70. The system of claim 67 or 68, wherein the one or more genes comprises a gene encoding a component of a Pol II transcription pathway.

71. The system of claim 70, wherein the one or more genes comprises one or more genes selected from the group consisting of GTF2H1, GTF2H4, GTF2F2, MNAT1, and CCNH.

72. The system of claim 67 or 68, wherein the one or more genes comprises a gene encoding a component of a ribonuclease P complex.

73. The system of claim 72, wherein the one or more genes comprises RPP21 and/or POP5.

74. A system for simultaneously editing both strands of a double-stranded DNA sequence at a target site to be edited, the system comprising:
(i) a first prime editor comprising a first nucleic acid programmable DNA binding protein (first napDNAbp) and a first polypeptide comprising an RNA-dependent DNA polymerase activity, or one or more polynucleotides encoding the first prime editor; and
(ii) a first prime editing guide RNA (first pegRNA) that binds to a first binding site on a first strand of the double-stranded DNA sequence upstream of the target site to be edited, or one or more polynucleotides encoding the first pegRNA;
(iii) a second prime editor comprising a second nucleic acid programmable DNA binding protein (second napDNAbp) and a second polypeptide comprising an RNA-dependent DNA polymerase activity, or one or more polynucleotides encoding the second prime editor; and
(iv) a second prime editing guide RNA (second pegRNA) that binds to a second binding site on a second strand of the double-stranded DNA sequence upstream of the target site to be edited, or one or more polynucleotides encoding the second pegRNA; and
(v) one or more inhibitors of one or more genes or gene products listed in Table 2 or Table X5;
wherein the first pegRNA comprises a first DNA synthesis template that comprises a region of complementary to the second binding site of the second pegRNA and the second pegRNA comprises a second DNA synthesis template that comprises a region of complementary to the first binding site of the first pegRNA.

75. A system for simultaneously editing both strands of a double-stranded DNA sequence at a target site to be edited, the system comprising:
(i) a first prime editor comprising a first nucleic acid programmable DNA binding protein (first napDNAbp) and a first polypeptide comprising an RNA-dependent DNA polymerase activity, or one or more polynucleotides encoding the first prime editor; and
(ii) a first prime editing guide RNA (first pegRNA) or one or more polynucleotides encoding the first pegRNA, wherein the first pegRNA comprises a first spacer sequence, a first gRNA core, a first DNA synthesis template, and a first primer binding site, wherein the first primer binding site binds to a first primer on a first strand of the double-stranded DNA sequence upstream of the target site to be edited;
(iii) a second prime editor comprising a second nucleic acid programmable DNA binding protein (second napDNAbp) and a second polypeptide comprising an RNA-dependent DNA polymerase activity, or one or more polynucleotides encoding the second prime editor; and
(iv) a second prime editing guide RNA (second pegRNA) or one or more polynucleotides encoding the second pegRNA, wherein the second pegRNA comprises a second spacer sequence, a second gRNA core, a second DNA synthesis template, and a second primer binding site, wherein the second primer binding site binds to a second primer on a second strand of the double-stranded DNA sequence upstream of the target site to be edited; and
(v) one or more inhibitors of one or more genes or gene products listed in Table 2 or Table X5,
wherein the first DNA synthesis template encodes a first single-stranded DNA, and the second DNA synthesis template encodes a second single-stranded DNA,
wherein the first single-stranded DNA sequence and the second single-stranded DNA sequence each comprise a region of complementarity to the other,
wherein the first single-stranded DNA sequence does have a region of complementarity to the second primer,
wherein the second single-stranded DNA sequence does have a region of complementarity to the first primer, and
wherein the first single-stranded DNA sequence and the second single-stranded DNA sequence form a duplex comprising an edited portion as compared to the DNA sequence at the target site to be edited.

76. The system of claim 74 or 75, wherein the one or more genes comprises one or more genes selected from the group consisting of XRN2, CHAF1B, UBE2T, DBR1, RAD17, FANCI, and FANCD2.

77. A system for editing a nucleic acid molecule by prime editing comprising a prime editor, a pegRNA, a ngRNA, and one or more inhibitors of one or more genes or gene products listed in Table 3 or Table X7, wherein the system is capable of installing one or more modification to the nucleic acid molecule at a target site.

78. The system of any one of claims 67-68, 74-75 and 77, wherein the one or more genes comprises a gene encoding a component of a H/ACA complex.

79. The system of claim 78, wherein the one or more genes comprises one or more genes selected from the group consisting of DKC1, NOP10, NHP2, and GAR1.

80. The system of any one of claims 67-68, 74-75 and 77, wherein the one or more genes comprises a gene encoding a component of a SMC5-SMC6 complex.

81. The system of claim 80, wherein the one or more genes comprises SMC5 or SMC6.

82. The system of any one of claims 67-68, 74-75 and 77, wherein the one or more genes comprises one or more genes selected from the group consisting of DBR1, XRN2, DKC1, NOP10, NHP2, GAR1, SMC5, SMC6, TALF6L, and ESPL1.

83. The system of any one of claims 67-68 and 74-75, wherein the one or more genes comprises one or more genes selected from the group consisting of RAD9A, RAD17, and CCNH.

84. The system of any one of claims 67-68 and 77, wherein the one or more genes comprises one or more genes selected from the group consisting of EXOSC2, EXOSC5, and SMC1A.

85. The system of any one of claims 74-75 and 77, wherein the one or more genes comprises KDM1A.

86. The system of any one of claims 67-69, wherein the inhibitor is a small molecule inhibitor, a siRNA, a microRNA, an antibody or antigen binding region, or a dominant negative of the one or more gene or gene products.

87. A method for editing a double-stranded DNA sequence by prime editing, the method comprising contacting the double-stranded DNA sequence with the system of any one of claims 66-86.


SEQUENCE LISTING (191 sequences)

SEQ ID NO: 1 - protein, 30 aa, synthetic construct
MDSLLMNRRKFLYQFKNVRWAKGRRETYLC

SEQ ID NO: 2 - protein, 1368 aa, Streptococcus pyogenes [sequence omitted]

SEQ ID NO: 3 - DNA, 120 bp, synthetic construct
  source 1..120: mol_type = other DNA
gtccaaagcaggatgacaggcaggggcaccgcggcgccccggtggcactgcggctggaggtgggggttaaagcggagactctggtgctgtgtgactacagtgggggccctgccctctctg

SEQ ID NO: 4 - DNA, 120 bp, synthetic construct
  source 1..120: mol_type = other DNA
gtccaaagcaggatgacaggcaggggcaccgcggcgccccggtggcactgcggctggaggggggggttaaagcggagactctggtgctgtgtgactacagtgggggccctgccctctctg

SEQ ID NO: 5 - DNA, 120 bp, synthetic construct
  source 1..120: mol_type = other DNA
gtccaaagcaggatgacaggcaggggcaccgcggcgccccggtggcactgcggctggaggatgacaggcaggggcaccgcggcgccccggtggcactgcggctggaggtgggggttaaag

SEQ ID NO: 6 - DNA, 120 bp, synthetic construct
  source 1..120: mol_type = other DNA
gtccaaagcaggatgacaggcaggggcaccgcggcgccccggtggcactgcggctggatgacaggcaggggcaccgcggcgccccggtggcactgcggctggaggtgggggttaaagcgg

SEQ ID NO: 7 - DNA, 120 bp, synthetic construct
  source 1..120: mol_type = other DNA
gtccaaagcaggatgacaggcaggggcaccgcggcgccccggtggcactgcggctggaggacaggcaggggcaccgcggcgccccggtggcactgcggctggaggtgggggttaaagcgg

SEQ ID NO: 8 - DNA, 120 bp, synthetic construct
  source 1..120: mol_type = other DNA
gtccaaagcaggatgacaggcaggggcaccgcggcgccccggtggcactgcggcaggatgacaggcaggggcaccgcggcgccccggtggcactgcggctggaggtgggggttaaagcgg

SEQ ID NO: 9 - DNA, 120 bp, synthetic construct
  source 1..120: mol_type = other DNA
gtccaaagcaggatgacaggcaggggcaccgcggcgccccggtggcactgcggctggaggtgggggttaaagcggcgactctggtgctgtgtgactacagtgggggccctgccctctctg

SEQ ID NO: 10 - DNA, 121 bp, synthetic construct
  source 1..121: mol_type = other DNA
gtccaaagcaggatggacaggcaggggcaccgcggcgccccggtggcactgcggctggaggtgggggttaaagcggagactctggtgctgtgtgactacagtgggggccctgccctctctg

SEQ ID NO: 11 - DNA, 119 bp, synthetic construct
  source 1..119: mol_type = other DNA
gtccaaagcaggatgacaggcaggggcaccgcggcgccccggtggcactgcggctggaggggggggttaaagcggagactctggtgctgtgtactacagtgggggccctgccctctctg

SEQ ID NO: 12 - DNA, 101 bp, synthetic construct
  source 1..101: mol_type = other DNA
gtccaaagcaggatgacaggcaggggcactgcggctggaggtgggggttaaagcggagactctggtgctgtgtgactacagtgggggccctgccctctctg

SEQ ID NO: 13 - DNA, 20 bp, synthetic construct
  source 1..20: mol_type = other DNA
gaccaggatgggcaccaccc

SEQ ID NO: 14-36 - skipped (23 entries)

SEQ ID NO: 37 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 38-80 - skipped (43 entries)

SEQ ID NO: 81 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 82-97 - skipped (16 entries)

SEQ ID NO: 98 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 99 - skipped

SEQ ID NO: 100 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 101 - protein, 19 aa, synthetic construct
MKRTADGSEFESPKKKRKV

SEQ ID NO: 102 - protein, 33 aa, synthetic construct
SGGSSGGSSGSETPGTSESATPESSGGSSGGSS

SEQ ID NO: 103 - protein, 21 aa, synthetic construct
SGGSKRTADGSEFEPKKKRKV

SEQ ID NO: 104-106 - skipped (3 entries)

SEQ ID NO: 107 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 108-117 - skipped (10 entries)

SEQ ID NO: 118 - protein, 150 aa, synthetic construct
  VARIANT 6..150: note = Up to 29 copies of GGGGS could be deleted
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 119 - protein, 30 aa, synthetic construct
  VARIANT 2..30: note = Up to 29 copies of G could be deleted
GGGGGGGGGGGGGGGGGGGGGGGGGGGGGG

SEQ ID NO: 120 - protein, 150 aa, synthetic construct
  VARIANT 6..150: note = Up to 29 copies of EAAAK could be deleted
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 121 - protein, 90 aa, synthetic construct
  VARIANT 4..90: note = Up to 29 copies of GGS could be deleted
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 122 - protein, 120 aa, synthetic construct
  VARIANT 5..120: note = Up to 29 copies of SGGS could be deleted
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 123 - protein, 60 aa, synthetic construct
  VARIANT 1..59: note = X is any amino acid
  VARIANT 3..60: note = Up to 29 copies of XP could be deleted
XPXPXPXPXPXPXPXPXPXPXPXPXPXPXPXPXPXPXPXPXPXPXPXPXPXPXPXPXPXP

SEQ ID NO: 124 - protein, 16 aa, synthetic construct
SGSETPGTSESATPES

SEQ ID NO: 125 - protein, 32 aa, synthetic construct
SGGSSGGSSGSETPGTSESATPESSGGSSGGS

SEQ ID NO: 126 - protein, 10 aa, synthetic construct
SGGSGGSGGS

SEQ ID NO: 127 - protein, 4 aa, synthetic construct
SGGS

SEQ ID NO: 128 - protein, 60 aa, synthetic construct
SGGSSGGSSGSETPGTSESATPESAGSYPYDVPDYAGSAAPAAKKKKLDGSGSGGSSGGS

SEQ ID NO: 129 - skipped

SEQ ID NO: 130 - protein, 6 aa, synthetic construct
GGSGGS

SEQ ID NO: 131 - protein, 9 aa, synthetic construct
GGSGGSGGS

SEQ ID NO: 132 - protein, 7 aa, synthetic construct
PKKKRKV

SEQ ID NO: 133 - protein, 20 aa, synthetic construct
AVKRPAATKKAGQAKKKKLD

SEQ ID NO: 134 - protein, 25 aa, synthetic construct
MSRRRKANPTKLSENAKKLAKEVEN

SEQ ID NO: 135 - protein, 9 aa, synthetic construct
PAAKRVKLD

SEQ ID NO: 136 - protein, 9 aa, synthetic construct
KLKIKRPVK

SEQ ID NO: 137 - protein, 8 aa, synthetic construct
VSRKRPRP

SEQ ID NO: 138 - protein, 10 aa, synthetic construct
EGAPPAKRAR

SEQ ID NO: 139 - protein, 12 aa, synthetic construct
PPQPKKKPLDGE

SEQ ID NO: 140 - protein, 18 aa, synthetic construct
KRTADGSEFESPKKKRKV

SEQ ID NO: 141 - protein, 17 aa, synthetic construct
KRTADGSEFEPKKKRKV

SEQ ID NO: 142 - protein, 19 aa, synthetic construct
NLSKRPAAIKKAGQAKKKK

SEQ ID NO: 143 - protein, 11 aa, synthetic construct
RQRRNELKRSF

SEQ ID NO: 144 - protein, 38 aa, synthetic construct
NQSSNFGPMKGGNFGGRSSGPYGGGGQYFAKPRNQGGY

SEQ ID NO: 145 - protein, 16 aa, synthetic construct
KRXXXXXXXXXXKKKL

SEQ ID NO: 146-171 - skipped (26 entries)

SEQ ID NO: 172 - protein, 410 aa, synthetic construct
MEAENAGSYSLQQAQAFYTFPFQQLMAEAPNMAVVNEQQMPEEVPAPAPAQEPVQEAPKGRKRKPRTTEPKQPVEPKKPVESKKSGKSAKSKEKQEKITDTFKVKRKVDRFNGVSEAELLTKTLPDILTFNLDIVIIGINPGLMAAYKGHHYPGPGNHFWKCLFMSGLSEVQLNHMDDHTLPGKYGIGFTNMVERTTPGSKDLSSKEFREGGRILVQKLQKYQPRIAVFNGKCIYEIFSKEVFGVKVKNLEFGLQPHKIPDTETLCYVMPSSSARCAQFPRAQDKVHYYIKLKDLRDQLKGIERNMDVQEVQYTFDLQLAQEDAKKMAVKEEKYDPGYEAAYGGAYGENPCSSEPCGFSSNGLIESVELRGESAFSGIPNGQWMTQSFTDQIPSFSNHCGTQEQEEESHA

SEQ ID NO: 173 - DNA, 23 bp, synthetic construct
  source 1..23: mol_type = other DNA
  variation 21: note = n can be anything
mmmmmmmmnnnnnnnnnnnnngg

SEQ ID NO: 174 - skipped

SEQ ID NO: 175 - DNA, 23 bp, synthetic construct
  source 1..23: mol_type = other DNA
  variation 21: note = n can be anything
mmmmmmmmmnnnnnnnnnnnngg

SEQ ID NO: 176 - skipped

SEQ ID NO: 177 - DNA, 27 bp, synthetic construct
  source 1..27: mol_type = other DNA
  variation 21..22: note = n can be anything
mmmmmmmmnnnnnnnnnnnnnnagaaw

SEQ ID NO: 178 - skipped

SEQ ID NO: 179 - DNA, 27 bp, synthetic construct
  source 1..27: mol_type = other DNA
  variation 21..22: note = n can be anything
mmmmmmmmmnnnnnnnnnnnnnagaaw

SEQ ID NO: 180 - skipped

SEQ ID NO: 181 - DNA, 25 bp, synthetic construct
  source 1..25: mol_type = other DNA
  variation 21: note = n can be anything
  variation 24: note = n can be anything
mmmmmmmmnnnnnnnnnnnnnggng

SEQ ID NO: 182 - skipped

SEQ ID NO: 183 - DNA, 25 bp, synthetic construct
  source 1..25: mol_type = other DNA
  variation 21: note = n can be anything
  variation 24: note = n can be anything
mmmmmmmmmnnnnnnnnnnnnggng

SEQ ID NO: 184 - skipped

SEQ ID NO: 185 - DNA, 125 bp, synthetic construct
  source 1..125: mol_type = other DNA
nnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 186 - DNA, 121 bp, synthetic construct
  source 1..121: mol_type = other DNA
nnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 187 - DNA, 109 bp, synthetic construct
  source 1..109: mol_type = other DNA
nnnnnnnnnnnnnnnnnnnngtttttgtactctcagaaatgcagaagctacaaagataaggcttcatgccgaaatcaacaccctgtcattttatggcagggtgtttttt

SEQ ID NO: 188 - DNA, 102 bp, synthetic construct
  source 1..102: mol_type = other DNA
nnnnnnnnnnnnnnnnnnnngttttagagctagaaatagcaagttaaaataaggctagtccgttatcaacttgaaaaagtggcaccgagtcggtgctttttt

SEQ ID NO: 189 - DNA, 88 bp, synthetic construct
  source 1..88: mol_type = other DNA
nnnnnnnnnnnnnnnnnnnngttttagagctagaaatagcaagttaaaataaggctagtccgttatcaacttgaaaaagtgttttttt

SEQ ID NO: 190 - DNA, 76 bp, synthetic construct
  source 1..76: mol_type = other DNA
nnnnnnnnnnnnnnnnnnnngttttagagctagaaatagcaagttaaaataaggctagtccgttatcatttttttt

SEQ ID NO: 191 - RNA, 82 nt, synthetic construct
  source 1..82: mol_type = other RNA
gttttagagctagaaatagcaagttaaaataaaggctagtccgttatcaacttgaaaaagtggcaccgagtcggtgcttttt